(19) United States
(12) Patent Application Publication
(10) Pub. No.: US 2023/0212647 A1
(43) Pub. Date: Jul. 6, 2023

(54) SYSTEMS AND METHODS FOR RAPID IDENTIFICATION OF PROTEINS

(71) Applicant: Seer, Inc., Redwood City, CA (US)

(72) Inventors: Omid C. FAROKHZAD, Redwood City, CA (US); Philip MA, Redwood City, CA (US); John E. BLUME, Redwood City, CA (US); Asim SIDDIQUI, Redwood City, CA (US)

(21) Appl. No.: 17/999,828

(22) PCT Filed: May 28, 2021
(86) PCT No.: PCT/US2021/034991

§ 371 Date: Nov. 23, 2022

Related U.S. Application Data
(60) Provisional application No. 63/032,437, filed on May 29, 2020.

(51) Int. Cl. C12Q 1/6806; C12Q 1/6869; C12Q 1/6837; C12Q 1/6825

(52) U.S. Cl. CPC C12Q 1/6806; C12Q 1/6825; C12Q 1/6837; C12Q 1/6869

(57) ABSTRACT

Disclosed herein are systems and methods of use thereof for coupling affinity reagents (e.g., in solution affinity reagents such as proteins, peptides, and nucleic acids and libraries of affinity reagents) with particles having coronas for rapid detection of proteins in a sample.

### CROSS-REFERENCE

This application claims the benefit of U.S. Provisional Pat. Application No. 63/032,437 filed May 29, 2020, which is incorporated by reference in its entirety.

### BACKGROUND

While probe-based biomolecule detection has been tailored for a wide range of diagnostic and analytical assays, their utility is often limited by low target molecule abundance and broad off-target interference in complex biological samples. Accordingly, many binding assays suffer from low precision and low sensitivity. While these parameters may be enhanced through selective sample enrichment, such enrichment methods are often slow, user intensive, and expensive. Thus, rapid and accurate biomolecule detection may currently be feasible only for a limited number of sample types and diagnostic purposes.

### SUMMARY

Recognized herein is a need for accurate biomolecule detection over broad concentration ranges. In many cases, probe-based analysis is limited by off-target effects from the biomolecular consortia of complex samples. Aspects of the present application provide a range of methods for extending the utility of probe (e.g., antibody or aptamer) based assays by selectively enriching portions of biological samples. In some cases, the enrichment comprises biomolecule corona formation on the surface of particles. The enriched portions of the biological sample may comprise a higher abundance of rare and biological state-specific biomolecules, potentially enhancing the accuracy of probe-based detection and analysis.

In various aspects, the present disclosure provides a method of assaying a protein in a sample, the method comprising: a) incubating a particle in the sample thereby adsorbing biomolecules from the sample onto the particle to form a biomolecular corona; b) incubating the particle with an affinity reagent, wherein the affinity reagent comprises (i) an affinity reagent and (ii) a barcode, wherein the affinity reagent or the affinity reagent is capable of binding to a biomolecule in the biomolecular corona and the barcode corresponds to the biomolecule bound by the affinity reagent or affinity reagent; and c) assaying for the presence or absence of the barcode, thereby assaying for the presence or absence of the biomolecule.

In some aspects, the affinity reagent is an antibody, a peptide, a nucleic acid affinity reagent, a Fab, a Fab2, an scFv, an aptamer, a polypeptide affinity reagent scaffold, or a chemical moiety. In some aspects, the polypeptide affinity reagent scaffold is an adnectin, avamer, abamer, affibody, or nanobody. In some aspects, the affinity reagent is present in a library comprising a plurality of affinity reagents. In some aspects, the library comprises from 50 to 10¹⁰ distinct affinity reagents. In some aspects, each affinity reagent of the library has a unique barcode. In some aspects, the library is a combinatorial DNA library. In some aspects, the library is a DNA encoded library. In some aspects, the barcode is a barcode nucleotide sequence.

In some aspects, the assaying of c) comprises sequencing the barcode nucleotide sequence. In some aspects, the assaying of c) comprises thermal cycling amplification. In some aspects, the barcode nucleotide sequence is amplified prior to the sequencing. In some aspects, the amplification is thermal cycling amplification. In some aspects, the thermal cycling amplification is PCR amplification. In some aspects, the amplification is isothermal amplification. In some aspects, the sequencing is next generation sequencing. In some aspects, the sequencing is nanopore sequencing.

In some aspects, the affinity reagent is from 1 nm to 15 nm in one dimension. In some aspects, the affinity reagent is from 200 Da to 200 kDa. In some aspects, the particle is from 5 nm to 50 um in one dimension. In some aspects, the one dimension is diameter.

In some aspects, the particle is organic, inorganic, a hybrid organic-inorganic particle, or polymeric particle. In some aspects, the particle is a hollow particle, a solid particle, a porous particle, or a multi-layered particle. In some aspects, the particle is a sphere, a rod, a triangle, a cylinder, a cube, or other geometrical or non-geometrical shape. In some aspects, the particle anionic, cationic, or neutral. In some aspects, the particle is surface modified with a small molecule, peptide, protein, antibody, aptamer, or a functional chemical group. In some aspects, the particle is a nanoparticle, a microparticle, a micelle, a liposome, an iron oxide particle, a graphene particle, a silica particle, a protein-based particle, a polystyrene particle, a silver particle, a gold particle, a quantum dot, a palladium particle, a platinum particle, a titanium particle, or any combinations thereof.

In some aspects, the affinity reagent and the barcode are coupled by a linker. In some aspects, the linker is C3 linker, a C6 linker, a C12 linker, a C18 linker, a C36 linker, a polypeptide linker, a chemical linker, a PEG linker, a cleavable linker, or a non-cleavable linker. In some aspects, the nucleic acid molecule is from 20 to 1000 nucleotides in length. In some aspects, the biomolecule is a protein.

In some aspects, the biomolecule is a lipid, a nucleic acid, a polysaccharide, or a protein. In some aspects, the sample comprises plasma, serum, urine, cerebrospinal fluid, synovial fluid, tears, saliva, whole blood, milk, nipple aspirate, ductal lavage, vaginal fluid, nasal fluid, ear fluid, gastric fluid, pancreatic fluid, trabecular fluid, lung lavage, sweat, crevicular fluid, semen, prostatic fluid, sputum, fecal matter, bronchial lavage, fluid from swabbings, bronchial aspirants, fluidized solids, fine needle aspiration samples, tissue homogenates, or cell culture. In some aspects, the affinity reagent comprises two or more affinity reagents. In some aspects, the affinity reagent comprises two affinity reagents directed to different regions of the same protein. In some aspects, the affinity reagent comprises two affinity reagents directed to two different proteins in close proximity. In some aspects, the two affinity reagents each comprise a nucleic acid that hybridize. In some aspects, the affinity reagent comprises one or more fluorophore.

Disclosed herein, in some aspects, are methods of assaying a biomolecule in a sample, the method comprising: a) incubating a particle in the sample thereby adsorbing biomolecules from the sample onto the particle to form a biomolecule corona; b) incubating the particle with an affinity reagent that binds to a biomolecule of the biomolecule corona; and c) assaying for the presence, absence or amount of the affinity reagent, thereby assaying for the presence, absence or amount of the biomolecule of the biomolecule corona. In some aspects, the affinity reagent comprises a nucleic acid. In some aspects, the affinity reagent comprises an aptamer. In some aspects, assaying for the presence, absence or amount of the affinity reagent comprises sequencing the aptamer. In some aspects, the aptamer binds comprises binding specificity for the biomolecule. In some aspects, the biomolecule is more abundant in a sample of a subject having a first biological state than in a sample of a subject having a second biological state. In some aspects, the affinity reagent has been subjected to error prone nucleic acid amplification. In some aspects, the affinity reagent is present in a plurality or library of affinity reagents.

Disclosed herein, in some aspects, are methods of assaying a biomolecule in a sample, the method comprising: a) incubating a particle in the sample thereby adsorbing biomolecules from the sample onto the particle to form a biomolecule corona; b) incubating the particle with a probe comprising an affinity reagent that binds to a biomolecule of the biomolecule corona; and c) assaying for the presence, absence or amount of the probe, thereby assaying for the presence, absence or amount of the biomolecule of the biomolecule corona. In some aspects, the probe comprises a detection modality. In some aspects, the detection modality is detectable optically, electrochemically, chemically, magnetically, chromatographically, by affinity capture, mass spectrometrically, or any combination thereof. In some aspects, the detection modality comprises a dye, a fluorescent tag, an electrochemically detectable tag, a magnetic tag, an affinity label, a polymer, a mass tag, or any combination thereof. In some aspects, the probe is present in a plurality of probes.

Disclosed herein, in some aspects, are methods of assaying a biomolecule in a sample, the method comprising: a) incubating a particle in the sample, thereby adsorbing biomolecules from the sample onto the particle to form a biomolecule corona; b) incubating the particle with a probe comprising (i) an affinity reagent and (ii) a barcode, wherein the affinity reagent binds to a biomolecule of the biomolecule corona; and c) assaying for the presence, absence or amount of the probe, thereby assaying for the presence, absence or amount of the biomolecule of the biomolecule corona. In some aspects, the affinity reagent comprises an antibody, a peptide, a nucleic acid ligand, a Fab, a Fab2, an scFv, an scFab, an aptamer, a polypeptide ligand scaffold, a ligand, or a chemical moiety. In some aspects, the peptide comprises an adnectin, abamer, affibody, or nanobody. In some aspects, the affinity reagent is from about 1 nm to about 35 nm in a dimension. In some aspects, the affinity reagent comprises a molecular mass from 200 Da to 200 kDa. In some aspects, the barcode comprises a single stranded nucleic acid, a double stranded nucleic acid, or a sticky end of a nucleic acid. In some aspects, the probe is present in a plurality of probes. In some aspects, the plurality of probes comprise different affinity reagents. In some aspects, the plurality of probes comprise a library of barcodes. In some aspects, each probe of the plurality of probes comprises a unique barcode. In some aspects, the library of barcodes comprises from 50 to 1010 distinct barcodes. In some aspects, the library of barcodes comprises a combinatorially generated nucleic acid library. In some aspects, the library of barcodes comprises double stranded DNA barcodes. In some aspects, the barcodes comprise barcode nucleotide sequences. In some aspects, affinity reagents of the plurality of probes bind different biomolecules, and wherein different biomolecules may be identified by the barcode nucleotide sequences of probes that bind to the different biomolecules. In some aspects, probes comprising affinity reagents that bind a biomolecule include a first barcode nucleotide sequence, and probes comprising affinity reagents that bind another biomolecule include a second barcode nucleotide sequence. In some aspects, a first probe of the plurality of probes comprises a first affinity reagent that binds a first biomolecule, and a second probe of the plurality comprises a second affinity reagent that binds a different region of the first biomolecule. In some aspects, a first probe of the plurality of probes comprises a first affinity reagent that binds a first biomolecule, and a second probe of the plurality of probes comprises a second affinity reagent that binds a second biomolecule in close proximity with the first biomolecule. In some aspects, a barcode of the first probe hybridizes with a barcode of the second probe. Some aspects include extending the 3′ ends of the hybridized barcodes of the first and second probes. In some aspects, the barcodes of the first and second probes comprise sticky ends that hybridize together, and further comprising ligating the sticky ends. In some aspects, the assaying of c) comprises sequencing the barcode nucleotide sequences. In some aspects, the barcode nucleotide sequences comprise primer sequences. In some aspects, the assaying of c) comprises amplification. In some aspects, the barcode nucleotide sequences or a segment of the barcode nucleotide sequences is amplified prior to sequencing. In some aspects, the amplification comprises thermal cycling amplification. In some aspects, the thermal cycling amplification comprises polymerase chain reaction. In some aspects, the amplification comprises isothermal amplification. In some aspects, the sequencing comprises next generation sequencing. In some aspects, the sequencing is nanopore sequencing. In some aspects, the particle is from 5 nm to 50 µm in a dimension. In some aspects, the dimension comprises a diameter. In some aspects, the particle comprises an organic, inorganic, hybrid organic-inorganic, or polymeric particle. In some aspects, the particle comprises a hollow particle, a solid particle, a porous particle, or a multi-layered particle. In some aspects, the particle comprises a sphere, a rod, a triangle, a cylinder, a cube, a low symmetry shape, or another geometrical shape. In some aspects, the particle comprises an anionic, cationic, or neutral charge. In some aspects, the particle is surface modified with a small molecule, peptide, protein, antibody, aptamer, or a functional chemical group. In some aspects, the particle comprises a nanoparticle, microparticle, micelle, liposome, iron oxide particle, graphene particle, silica particle, protein-based particle, polystyrene particle, silver particle, gold particle, quantum dot, palladium particle, platinum particle, titanium particle, or any combinations thereof. In some aspects, the probe comprises a fluorophore. In some aspects, the probe and the barcode are conjugated by a linker. In some aspects, the linker comprises a C3 linker, a C6 linker, a C12 linker, a C18 linker, a C36 linker, a peptide linker, a nucleic acid linker, a chemical linker, a PEG linker, a cleavable linker, or a non-cleavable linker. In some aspects, the barcode comprises a nucleic acid molecule from 20 to 1000 nucleotides in length. In some aspects, the biomolecule comprises a protein. In some aspects, the protein comprises a post-translational modification recognizable by the affinity reagent. In some aspects, the biomolecule comprises a lipid, a nucleic acid, or a saccharide. In some aspects, the sample comprises a biofluid. In some aspects, the biofluid comprises plasma, serum, urine, cerebrospinal fluid, synovial fluid, tears, saliva, whole blood, milk, nipple aspirate, ductal lavage, vaginal fluid, nasal fluid, ear fluid, gastric fluid, pancreatic fluid, trabecular fluid, lung lavage, sweat, crevicular fluid, semen, prostatic fluid, sputum, fecal matter, bronchial lavage, fluid from a swabbing, or a bronchial aspirant. In some aspects, the sample comprises a fluidized solid, a tissue homogenate, or a cultured cell. Some aspects include performing a wash step after a) to wash away biomolecules not adsorbed to the particle, performing a wash step after b) to wash away unbound probes, or performing a combination of wash steps. In some aspects, the assaying of c) comprises separating the probe from the biomolecule. In some aspects, the assaying of c) comprises separating the barcode from the affinity reagent. In some aspects, the assaying of c) comprises measuring a readout indicative of the presence, absence or amount of the barcode. In some aspects, the assaying of c) comprises assaying for the presence or absence of the barcode. In some aspects, the assaying of c) comprises assaying for an amount of the barcode. In some aspects, the barcode corresponds to the biomolecule bound by the affinity reagent. Some aspects include contacting the probe with a secondary probe comprising a nucleotide that hybridizes with the barcode. In some aspects, the secondary probe comprises a detection modality. In some aspects, the detection modality of the secondary probe is fluorescent. In some aspects, c) comprises measuring a readout indicative of the presence, absence or amount of the detection modality of the secondary probe. In some aspects, the secondary probe is present in a plurality of secondary probes comprising different tags and nucleotides that hybridize with different barcode sequences. Some aspects include performing mass spectrometry, chromatography, liquid chromatography, high-performance liquid chromatography, solid-phase chromatography, a lateral flow assay, an immunoassay, an enzyme-linked immunosorbent assay, a western blot, a dot blot, or immunostaining, or a combination thereof, on the biomolecule of the biomolecule corona or on one or more other biomolecules of the biomolecule corona. In some aspects, the affinity reagent comprises the barcode.

Disclosed herein, in some aspects, are methods of assaying biomolecules, comprising: a) incubating a particle in a biological sample, thereby adsorbing biomolecules from the biological sample onto the particles to form biomolecule coronas; b) incubating the particles with probes comprising (i) affinity reagents and (ii) barcodes, wherein the affinity reagents bind to biomolecules of the biomolecule coronas; c) detecting the presence or amount of the barcodes of the probes comprising affinity reagents bound to biomolecules of the biomolecule coronas; and d) identifying a biomolecule fingerprint associated with the biological sample based on the presence or amount of the barcodes. Some aspects include identifying the presence or amount of the biomolecules of the biomolecule coronas based on the presence or amount of the barcodes. In some aspects, identifying the biomolecule fingerprint associated with the biological sample based on the presence or amount of the barcodes comprises identifying the biomolecule fingerprint based on the presence or amount of the biomolecules of the biomolecule coronas. Some aspects include identifying a disease state associated with the biomolecule fingerprint. In some aspects, the disease state comprises a cancer, cardiovascular disease, endocrine disease, inflammatory disease, or neurological disease. In some aspects, identifying the disease state associated with the biomolecule fingerprint comprises applying a classifier to the biomolecule fingerprint. In some aspects, the classifier has been trained with data comprising the presence or amounts of barcodes of probes bound to biomolecule coronas of healthy or diseased subjects. In some aspects, the particles comprise physiochemically distinct groups of particles.

Disclosed herein, in some aspects, are methods of assaying a biomolecule in a sample, the method comprising: a) incubating a particle in the sample thereby adsorbing biomolecules from the sample onto the particle to form a biomolecule corona; b) desorbing biomolecules of the biomolecule corona from the particle; c) contacting the desorbed biomolecules with a probe comprising (i) an affinity reagent and (ii) a detection modality, wherein the affinity reagent binds to a biomolecule of the desorbed biomolecules; and d) assaying for the presence, absence or amount of the detection modality of the probe comprising the affinity reagent, thereby assaying for the presence, absence or amount of the biomolecule of the desorbed biomolecules. In some aspects, the detection modality comprises a barcode. Some aspects include binding the desorbed biomolecules to a substrate prior to d). In some aspects, the substrate has a flat surface to which the desorbed biomolecules are bound. In some aspects, the desorbed biomolecules are bound indirectly to the substrate. In some aspects, the desorbed biomolecules are bound to the substrate by capture moieties. In some aspects, the probe is bound to the substrate. Some aspects include releasing the desorbed biomolecules from being bound to the substrate prior to d). In some aspects, the substrate comprises glass, a polymer, rubber, plastic, or a metal. Some aspects include releasing the desorbed biomolecules from being bound to the probe prior to d). In some aspects, d) comprises assaying for the presence, absence or amount of the detection modality of the probe comprising the affinity reagent bound to the biomolecule of the desorbed biomolecules.

Disclosed herein, in some aspects, are methods, comprising: a) incubating a particle in a sample, thereby adsorbing biomolecules from the sample onto the particle to form a biomolecule corona; b) incubating the biomolecules of the biomolecule corona with a substrate of a biomolecule of the biomolecule corona; and c) measuring a reaction product of the substrate, thereby assaying for a presence, absence, or an amount of the biomolecule of the biomolecule corona. Some aspects include incubating the particle with a probe comprising an affinity reagent that binds to the biomolecule of the biomolecule corona, and blocks formation of the reaction product from the substrate. In some aspects, the probe further comprises a barcode nucleotide sequence. Some aspects include incubating sequencing the barcode. Some aspects include incubating identifying the affinity reagent as an inhibitor of an enzyme activity of the biomolecule, based on the sequencing of the barcode.

Disclosed herein, in some aspects, are methods, comprising: a) flowing a sample over or through a matrix, thereby adsorbing biomolecules from the sample onto the matrix; b) flowing a probe over or through the matrix, wherein the probe comprises (i) an affinity reagent and (ii) a barcode, and wherein the affinity reagent binds to a biomolecule of the adsorbed biomolecules; and c) assaying for the presence, absence or amount of the probe, thereby assaying for the presence, absence or amount of the biomolecule of the adsorbed biomolecules. In some aspects, the matrix is semipermeable. In some aspects, the matrix comprises a porous material. In some aspects, the matrix comprises a property comprising a charge, a hydrophobicity, or a surface functionalization.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “figure” and “FIG.” herein), of which:

FIG. 1 shows a computer system that is programmed or otherwise configured to implement methods provided herein.

FIG. 2 provides an example workflow for collecting biomolecules from a biological sample onto particles.

FIG. 3 provides an example workflow for a particle-based assay for analyzing biomolecules from a biological sample.

FIG. 4 provides an example workflow for assaying biomolecules from a biological sample with magnetic particles.

FIG. 5 illustrates numbers of proteins collected on and subsequently identified by mass spectrometry following collection on particle panels comprising from 1 to 12 particles.

FIG. 6 provide a schematic workflow for an affinity reagent analysis method consistent with the present disclosure.

FIG. 7 illustrates a non-limiting, hypothetical example of a proteome analysis method that combines biomolecule corona analysis with a probe (e.g., a DNA encoded library (DEL)) binding assay.

FIG. 8 outlines a non-limiting, hypothetical example of a method for assaying a sample with probes comprising broad binding specificities.

FIG. 9 illustrates a non-limiting, hypothetical example of a method for assaying a sample by contacting the sample with a library of probes with known target binding specificities.

FIG. 10 illustrates a non-limiting, hypothetical example of a parallelized multi-particle assay comprising affinity reagent analysis.

FIG. 11 shows a schematic for a non-limiting, hypothetical example of a proteome analysis method that combines biomolecule corona analysis with a probe binding assay.

FIG. 12 provides a non-limiting, hypothetical example of a method for measuring inter-biomolecule distances in a biomolecule corona with barcode-containing probes.

FIG. 13 provides a non-limiting, hypothetical example of a method involving biomolecule collection on particles and a proximity extension assay.

FIG. 14 illustrates a non-limiting, hypothetical example of a biomolecule corona-based proximity extension assay.

FIG. 15 provides a non-limiting, hypothetical example of a DNA-encoded library binding assay comprising cleavage and analysis of DNA barcodes from probes bound to a biomolecule corona.

FIG. 16 illustrates a non-limiting, hypothetical example of a method for analyzing a biomolecule corona with a library of nucleic acid barcoded probes and detection modalities configured to bind to the nucleic acid barcodes.

FIG. 17 provides a schematic for a non-limiting, hypothetical example of a proximity ligation assay performed on a biomolecule corona.

FIG. 18 provides a non-limiting, hypothetical example of an aptamer library directed evolution method in which a library of aptamer probes comprising nucleic acid molecules are subjected to rounds of positive and negative selection.

FIG. 19 outlines a non-limiting, hypothetical example of a method for training a classifier to distinguish between multiple sample types based on differential probe binding.

FIG. 20 provides a non-limiting, hypothetical example of a method for identifying enzyme inhibitors or elucidating enzyme activity by interrogating probe binding to a biomolecule corona.

FIG. 21 illustrates a non-limiting, hypothetical example of an affinity reagent library evolution method that utilizes biomolecule corona analysis.

FIG. 22 illustrates a non-limiting, hypothetical example of a method for assaying a sample with a sensor element and a probe library.

FIG. 23 illustrates a non-limiting, hypothetical example of a well plate, as well as a method for using the well plate to assay a sample.

FIG. 24 provides a non-limiting, hypothetical example of a multi-condition biomolecule corona assay consistent with the present disclosure.

### DETAILED DESCRIPTION

Biological samples are often complex mixtures of biomolecules with concentrations spanning orders of magnitude and comprising disparate properties. Accordingly, detecting a broad subset of biomolecules from a sample is often challenging, time intensive, and limited in terms of accuracy and breadth. The present disclosure provides a range of methods for fractionating, collecting, and enriching biomolecules from complex biological samples, thereby enabling deep analysis, profiling, and biomolecule detection.

In various aspects, the present disclosure provides a method of assaying a biomolecule in a sample, the method comprising: incubating a particle in the sample, thereby adsorbing the biomolecule onto the particle; incubating the sample with a probe comprising an affinity reagent, thereby binding the affinity reagent to the biomolecule; and assaying for the probe, thereby assaying for the biomolecule. A complex may be formed comprising the affinity reagent bound to the biomolecule adsorbed to the particle. Assaying for the probe may include assaying for the probe bound to the affinity reagent. The biomolecule may be part of a plurality of biomolecules that adsorb from the sample onto the particle to form a biomolecular corona.

Aspects of the present disclosure provide compositions, systems, and methods for collecting biomolecules on particles. Particle panels of multiple distinct particle types, which enrich proteins from a sample onto distinct biomolecule coronas formed on the surface of the distinct particle types. The particle panels disclosed herein can be used in methods of corona analysis to detect thousands of proteins across a wide dynamic range in the span of hours.

Aspects of the present disclosure provide methods for analyzing peptides. As used herein, ‘peptide’ may refer to a molecule comprising at least two amino acid residues linked by peptide (e.g., amide) bonds. The term ‘peptide’ may refer to amino acid dimers, trimers, oligomers, or polymers. The term ‘peptide’ may also refer to a protein. A peptide may be linear or branched. A peptide may comprise a natural amino acid. A natural amino acid may be a ‘proteinogenic amino acid’, which, as used herein, may refer to any one of the 22 known amino acids utilized for translation by natural organisms, namely alanine, arginine, asparagine, aspartic acid, cystine, glutamic acid, glutamine, glycine, histidine, isoleucine, leucine, lysine, methionine, phenylalanine, proline, serine, threonine, tryptophan, tyrosine, valine, pyrrolysine, and selenocystine. A natural amino acid may be a post-translationally modified amino acid, nonlimiting examples of which include acylated amino acids, alkylated amino acids, prenylated amino acids, nitrosylated amino acids, flavinated amino acids, formylated amino acids, amidated amino acids, deamidated amino acids, halogenated amino acids, carboxylated amino acids, decarboxylated amino acids, glycosylated amino acids, phosphorylated amino acids, sulfurylated amino acids, cyclized amino acids, carbamylated amino acids, carbonylated amino acids, or biotinylated amino acids. A peptide may comprise an isomeric variant of a naturally occurring amino acid, such as an α-carbon enantiomer, also known as a D-amino acid. A peptide may comprise a non-natural (e.g., synthetically derived) amino acid. A non-natural amino acid may comprise a non-natural side chain, such as a perfluorinated aryl or alkyl moiety. A non-natural amino acid may comprise a non-natural backbone structure, for example a silicon in place of the α-carbon or the amine disposed on a β-carbon. A peptide may also comprise non-amino acid units, such as 4-hydroxybutanoic, in place of amino acid residues.

A method of the present disclosure may comprise contacting a biological sample (e.g., plasma) with a particle under conditions suitable for biomolecule collection (e.g., non-covalent adsorption) on the particle. The collection of biomolecules on the surface of the particle may be referred to as a ‘biomolecule corona’. The biomolecule corona that forms on a particle may comprise a complex mixture of biomolecules from the biological sample. The biomolecule corona may compress the abundance ratios of biomolecules from a sample, thereby enabling analysis of dilute, and in many cases difficult to analyze, biomolecules. A biomolecule corona may include nucleic acids, small molecules, proteins, lipids, polysaccharides, or any combination thereof, adsorbed to the surface of a particle form a sample in which the particle is incubated. nucleic acid, a small molecule, a protein, a lipid, a polysaccharide, or any combination thereof.

Accurately profiling complex chemical profiles is a longstanding problem in a wide range of disciplines. Individual assaying techniques can be limited by narrow dynamic measurement ranges, the inability to distinguish between similar molecules (e.g., protein splice variants), and the inability to simultaneously measure chemically disparate species. Direct sample analysis with affinity reagents is often limited to high specificity affinity reagents (e.g., monospecific antibodies) which experience limited effects from the complex biomolecular consortia of complex samples. Conversely, particle-based analysis sometimes requires time-intensive purification and analyte analysis steps following biomolecule corona formation, which can make certain methods impractical for routine use. Accordingly, the present disclosure provides a range of strategies for profiling complex biological samples with combinations of particles and affinity reagents.

The present invention described herein provides particles that collect subsets of biomolecules from complex biological samples, and probes that selectively bind to biomolecules of interest. The particles and probes may be combined to obtain extensive information on the chemical and physical makeup of a sample. A particle may be used to enrich a subset of biomolecules (e.g., a biomolecule corona) from a biological sample for interrogation with a probe. Contacting an affinity reagent to a biomolecule corona of a particle, rather than to a complex biological sample, may diminish off-target binding and interference by high abundance biomolecules, and may further enable the use of probes with broad binding-specificities. Such an enriched sample may comprise an increased abundance of relevant (e.g., disease-specific) biomolecules, or may decrease the prevalence of off-target biomolecules which interfere with probe-target binding. Accordingly, a tandem particle and probe assay may facilitate biomolecule profiling to a depth not achievable with conventional methods.

### Particle Types and Properties

The present disclosure provides a range of strategies for enriching subsets of biomolecules from complex biological samples. The compositions, systems, and methods disclosed herein may utilize a particle or a combination of particles (referred to hereinafter as particle panels) having one or more different particle types, which may be incubated with a sample to form biomolecule coronas. Particles may comprise surfaces which selectively enrich subsets of biomolecules from complex samples. In some cases, a particle may comprise a surface which preferentially binds low abundance biomolecules from a biological sample. For example, a particle may generate a biomolecule corona from plasma with an enriched abundance of cytokines relative to albumin and globulins.

“Biomolecule corona” as used herein can be used referred to interchangeably with the term “protein corona,” and refers to the formation of a layer of biomolecules on the surface of a particle after the particle has been contacted with a sample (e.g., plasma). This method may be referred to interchangeably as corona analysis or, in some examples, “Proteograph” analysis, which combines a multi-particle type protein corona strategy with mass spectrometry (MS). Particle types included in the particle panels disclosed herein can be superparamagnetic and are, thus, rapidly separated or isolated from unbound protein (proteins that have not adsorbed onto the surface of a particle to form the corona) in a sample, after incubation of the particle in the sample.

Aspects of the present disclosure provide particle panels comprising pluralities of particles which differentially enrich biomolecules from complex biological samples. The particle types included in the particle panels disclosed herein are particularly well suited for enriching large numbers of proteins across wide dynamic ranges. The combinations of particle types selected for a particle panel of the present disclosure may be varied in their physicochemical properties (e.g., size, surface charge, core material, shell material, surface chemistry, porosity, morphology, and other properties). However, particle types may also share physicochemical properties in common. For example, a plurality of particles may share a common surface functionalization (e.g., amine functionalization), a common core material (e.g., iron oxide), or a common shell material (e.g., polystyrene).

Particles can be used combinatorially in the methods disclosed herein of rapidly identifying proteins. Particle types consistent with the methods disclosed herein can be made from various materials. For example, particle materials consistent with the present disclosure include metals, polymers, magnetic materials, and lipids. Particles consistent with the present disclosure may be organic or inorganic. Magnetic particles may be iron oxide particles. Examples of metal materials include any one of or any combination of gold, silver, copper, nickel, cobalt, palladium, platinum, iridium, osmium, rhodium, ruthenium, rhenium, vanadium, chromium, manganese, niobium, molybdenum, tungsten, tantalum, iron and cadmium, or any other material described in US7749299. In some embodiments, a particle may be a superparamagnetic iron oxide nanoparticle (SPION).

A particle may comprise a polymeric core, layer, shell, or combination thereof. A particle may be entirely comprised of a polymer or a plurality of polymers. Examples of polymers include any one of or any combination of polyethylenes, polycarbonates, polyanhydrides, polyhydroxyacids, polypropylfumerates, polycaprolactones, polyamides, polyacetals, polyethers, polyesters, poly(orthoesters), polycyanoacrylates, polyvinyl alcohols, polyurethanes, polyphosphazenes, polyacrylates, polymethacrylates, polycyanoacrylates, polyureas, polystyrenes, or polyamines, a polyalkylene glycol (e.g., polyethylene glycol (PEG)), a polyester (e.g., poly(lactide-co-glycolide) (PLGA), polylactic acid, or polycaprolactone), or a copolymer of two or more polymers, such as a copolymer of a polyalkylene glycol (e.g., PEG) and a polyester (e.g., PLGA). In some embodiments, the polymer is a lipid-terminated polyalkylene glycol and a polyester, or any other material disclosed in US9549901.

A particle may comprise a lipid. The lipid may be covalently (e.g., covalently bound to a silica particle coating) or non-covalently coupled to a particle. The lipid may be present within a micelle or liposome of a particle. Examples of lipids that can be used to form the particles of the present disclosure include cationic, anionic, and neutrally charged lipids. For example, particles can be made of any one of or any combination of dioleoylphosphatidylglycerol (DOPG), diacylphosphatidylcholine, diacylphosphatidylethanolamine, ceramide, sphingomyelin, cephalin, cholesterol, cerebrosides and diacylglycerols, dioleoylphosphatidylcholine (DOPC), dimyristoylphosphatidylcholine (DMPC), and dioleoylphosphatidylserine (DOPS), phosphatidylglycerol, cardiolipin, diacylphosphatidylserine, diacylphosphatidic acid, N-dodecanoyl phosphatidylethanolamines, N-succinyl phosphatidylethanolamines, N-glutarylphosphatidylethanolamines, lysylphosphatidylglycerols, palmitoyloleyolphosphatidylglycerol (POPG), lecithin, lysolecithin, phosphatidylethanolamine, lysophosphatidylethanolamine, dioleoylphosphatidylethanolamine (DOPE), dipalmitoyl phosphatidyl ethanolamine (DPPE), dimyristoylphosphoethanolamine (DMPE), distearoyl-phosphatidyl-ethanolamine (DSPE), palmitoyloleoyl-phosphatidylethanolamine (POPE) palmitoyloleoylphosphatidylcholine (POPC), egg phosphatidylcholine (EPC), distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), palmitoyloleyolphosphatidylglycerol (POPG), 16-O-monomethyl PE, 16-O-dimethyl PE, 18-1-trans PE, palmitoyloleoyl-phosphatidylethanolamine (POPE), 1-stearoyl-2-oleoyl-phosphatidyethanolamine (SOPE), phosphatidylserine, phosphatidylinositol, sphingomyelin, cephalin, cardiolipin, phosphatidic acid, cerebrosides, dicetylphosphate, and cholesterol, or any other material listed in US9445994, which is incorporated herein by reference in its entirety.

A particle of the present disclosure may be synthesized, or a particle of the present disclosure may be purchased from a commercial vendor. For example, particles consistent with the present disclosure may be purchased from commercial vendors including Sigma-Aldrich, Life Technologies, Fisher Biosciences, nanoComposix, Nanopartz, Spherotech, and other commercial vendors. In some embodiments, a particle of the present disclosure may be purchased from a commercial vendor and further modified, coated, or functionalized.

Particles consistent with the present disclosure can include nanoparticles and microparticles. Particles that are consistent with the present disclosure can be made and used in methods of forming protein coronas after incubation in a sample at a wide range of sizes. In some embodiments, a particle of the present disclosure may be a nanoparticle. In some embodiments, a nanoparticle of the present disclosure may be from about 10 nm to about 1000 nm in diameter. For example, the nanoparticles disclosed herein can be at least 10 nm, at least 100 nm, at least 200 nm, at least 300 nm, at least 400 nm, at least 500 nm, at least 600 nm, at least 700 nm, at least 800 nm, at least 900 nm, from 10 nm to 50 nm, from 50 nm to 100 nm, from 100 nm to 150 nm, from 150 nm to 200 nm, from 200 nm to 250 nm, from 250 nm to 300 nm, from 300 nm to 350 nm, from 350 nm to 400 nm, from 400 nm to 450 nm, from 450 nm to 500 nm, from 500 nm to 550 nm, from 550 nm to 600 nm, from 600 nm to 650 nm, from 650 nm to 700 nm, from 700 nm to 750 nm, from 750 nm to 800 nm, from 800 nm to 850 nm, from 850 nm to 900 nm, from 100 nm to 300 nm, from 150 nm to 350 nm, from 200 nm to 400 nm, from 250 nm to 450 nm, from 300 nm to 500 nm, from 350 nm to 550 nm, from 400 nm to 600 nm, from 450 nm to 650 nm, from 500 nm to 700 nm, from 550 nm to 750 nm, from 600 nm to 800 nm, from 650 nm to 850 nm, from 700 nm to 900 nm, or from 10 nm to 900 nm in diameter. In some embodiments, a nanoparticle may be less than 1000 nm in diameter.

In some embodiments, a particle of the present disclosure may be a microparticle. A microparticle may be a particle that is from about 1 µm to about 1000 µm in diameter. For example, the microparticles disclosed here can be at least 1 µm, at least 10 µm, at least 100 µm, at least 200 µm, at least 300 µm, at least 400 µm, at least 500 µm, at least 600 µm, at least 700 µm, at least 800 µm, at least 900 µm, from 10 µm to 50 µm, from 50 µm to 100 µm, from 100 µm to 150 µm, from 150 µm to 200 µm, from 200 µm to 250 µm, from 250 µm to 300 µm, from 300 µm to 350 µm, from 350 µm to 400 µm, from 400 µm to 450 µm, from 450 µm to 500 µm, from 500 µm to 550 µm, from 550 µm to 600 µm, from 600 µm to 650 µm, from 650 µm to 700 µm, from 700 µm to 750 µm, from 750 µm to 800 µm, from 800 µm to 850 µm, from 850 µm to 900 µm, from 100 µm to 300 µm, from 150 µm to 350 µm, from 200 µm to 400 µm, from 250 µm to 450 µm, from 300 µm to 500 µm, from 350 µm to 550 µm, from 400 µm to 600 µm, from 450 µm to 650 µm, from 500 µm to 700 µm, from 550 µm to 750 µm, from 600 µm to 800 µm, from 650 µm to 850 µm, from 700 µm to 900 µm, or from 10 µm to 900 µm in diameter. In some embodiments, a microparticle may be less than 1000 µm in diameter.

An example of a particle type of the present disclosure may be a carboxylate (Citrate) superparamagnetic iron oxide nanoparticle (SPION), a phenol-formaldehyde coated SPION, a silica-coated SPION, a polystyrene coated SPION, a carboxylated poly(styrene-co-methacrylic acid) coated SPION, a N-(3-Trimethoxysilylpropyl)diethylenetriamine coated SPION, a poly(N-(3-(dimethylamino)propyl) methacrylamide) (PDMAPMA)-coated SPION, a 1,2,4,5-Benzenetetracarboxylic acid coated SPION, a poly(Vinylbenzyltrimethylammonium chloride) (PVBTMAC) coated SPION, a carboxylate, PAA coated SPION, a poly(oligo(ethylene glycol) methyl ether methacrylate) (POEGMA)-coated SPION, a carboxylate microparticle, a polystyrene carboxyl functionalized particle, a carboxylic acid coated particle, a silica particle, a carboxylic acid particle of about 150 nm in diameter, an amino surface microparticle of about 0.4-0.6 µm in diameter, a silica amino functionalized microparticle of about 0.1-0.39 µm in diameter, a Jeffamine surface particle of about 0.1-0.39 µm in diameter, a polystyrene microparticle of about 2.0-2.9 µm in diameter, a silica particle, a carboxylated particle with an original coating of about 50 nm in diameter, a particle coated with a dextran based coating of about 0.13 µm in diameter, or a silica silanol coated particle with low acidity.

Particle types consistent with the methods disclosed herein can be made from various materials. For example, particle materials consistent with the present disclosure include metals, polymers, magnetic materials, and lipids. Magnetic particles may be iron oxide particles. Examples of metal materials include any one of or any combination of gold, silver, copper, nickel, cobalt, palladium, platinum, iridium, osmium, rhodium, ruthenium, rhenium, vanadium, chromium, manganese, niobium, molybdenum, tungsten, tantalum, iron and cadmium, or any other material described in US7749299. A particle consistent with the compositions and methods disclosed herein may be a magnetic particle, such as a superparamagnetic iron oxide nanoparticle (SPION). A magnetic particle may be a ferromagnetic particle, a ferrimagnetic particle, a paramagnetic particle, a superparamagnetic particle, or any combination thereof (e.g., a particle may comprise a ferromagnetic material and a ferrimagnetic material). A particle may comprise a distinct core (e.g., the innermost portion of the particle), shell (e.g., the outermost layer of the particle), and shell or shells (e.g., portions of the particle disposed between the core and the shell). A particle may comprise a uniform composition.

A particle may comprise a polymer. The polymer may constitute a core material (e.g., the core of a particle may comprise a particle), a layer (e.g., a particle may comprise a layer of a polymer disposed between its core and its shell), a shell material (e.g., the surface of the particle may be coated with a polymer), or any combination thereof. Examples of polymers include any one of or any combination of polyethylenes, polycarbonates, polyanhydrides, polyhydroxyacids, polypropylfumerates, polycaprolactones, polyamides, polyacetals, polyethers, polyesters, poly(orthoesters), polycyanoacrylates, polyvinyl alcohols, polyurethanes, polyphosphazenes, polyacrylates, polymethacrylates, polycyanoacrylates, polyureas, polystyrenes, or polyamines, a polyalkylene glycol (e.g., polyethylene glycol (PEG)), a polyester (e.g., poly(lactide-co-glycolide) (PLGA), polylactic acid, or polycaprolactone), or a copolymer of two or more polymers, such as a copolymer of a polyalkylene glycol (e.g., PEG) and a polyester (e.g., PLGA). The polymer may comprise a cross link. A plurality of polymers in a particle may be phase separated, or may comprise a degree of phase separation. The polymer may comprise a lipid-terminated polyalkylene glycol and a polyester, or any other material disclosed in US9549901.

Examples of lipids that can be used to form the particles of the present disclosure include cationic, anionic, and neutrally charged lipids. For example, particles can be made of any one of or any combination of dioleoylphosphatidylglycerol (DOPG), diacylphosphatidylcholine, diacylphosphatidylethanolamine, ceramide, sphingomyelin, cephalin, cholesterol, cerebrosides and diacylglycerols, dioleoylphosphatidylcholine (DOPC), dimyristoylphosphatidylcholine (DMPC), and dioleoylphosphatidylserine (DOPS), phosphatidylglycerol, cardiolipin, diacylphosphatidylserine, diacylphosphatidic acid, N-dodecanoyl phosphatidylethanolamines, N-succinyl phosphatidylethanolamines, N-glutarylphosphatidylethanolamines, lysylphosphatidylglycerols, palmitoyloleyolphosphatidylglycerol (POPG), lecithin, lysolecithin, phosphatidylethanolamine, lysophosphatidylethanolamine, dioleoylphosphatidylethanolamine (DOPE), dipalmitoyl phosphatidyl ethanolamine (DPPE), dimyristoylphosphoethanolamine (DMPE), distearoyl-phosphatidyl-ethanolamine (DSPE), palmitoyloleoyl-phosphatidylethanolamine (POPE) palmitoyloleoylphosphatidylcholine (POPC), egg phosphatidylcholine (EPC), distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), palmitoyloleyolphosphatidylglycerol (POPG), 16-O-monomethyl PE, 16-O-dimethyl PE, 18-1-trans PE, palmitoyloleoyl-phosphatidylethanolamine (POPE), 1-stearoyl-2-oleoyl-phosphatidyethanolamine (SOPE), phosphatidylserine, phosphatidylinositol, sphingomyelin, cephalin, cardiolipin, phosphatidic acid, cerebrosides, dicetylphosphate, and cholesterol, or any other material listed in US9445994, which is incorporated herein by reference in its entirety. Examples of particles of the present disclosure are provided in TABLE 1.

TABLE 1

Example particles of the present disclosure

| | | | |
|---|---|---|---|
| Batch No. | Type | Particle ID | Description |
| S-001-001 | HX-13 | SP-001 | Carboxylate (Citrate) superparamagnetic iron oxide NPs (SPION) |
| S-002-001 | HX-19 | SP-002 | Phenol-formaldehyde coated SPION |
| S-003-001 | HX-20 | SP-003 | Silica-coated superparamagnetic iron oxide NPs (SPION) |
| S-004-001 | HX-31 | SP-004 | Polystyrene coated SPION |
| S-005-001 | HX-38 | SP-005 | Carboxylated Poly(styrene-co-methacrylic acid), P(St-co-MAA) coated SPION |
| S-006-001 | HX-42 | SP-006 | N-(3-Trimethoxysilylpropyl)diethylenetriamine coated SPION |
| S-007-001 | HX-56 | SP-007 | poly(N-(3-(dimethylamino)propyl) methacrylamide) (PDMAPMA)-coated SPION |
| S-008-001 | HX-57 | SP-008 | 1,2,4,5-Benzenetetracarboxylic acid coated SPION |
| S-009-001 | HX-58 | SP-009 | PVBTMAC coated poly(vinylbenzyltrimethylammonium chloride) (PVBTMAC) coated SPION |
| S-010-001 | HX-59 | SP-010 | Carboxylate, PAA coated SPION |
| S-011-001 | HX-86 | SP-011 | poly(oligo(ethylene glycol) methyl ether methacrylate) (POEGMA)-coated SPION |
| S-163-001 | S-163 | S-163 | Cis-ubiquitin-functionalized styrene particle |
| S-164-001 | S-164 | S-164 | Ubiquitin-functionalized styrene particle |
| P-033-001 | P33 | SP-333 | Carboxylate functionalized 1 µm magnetic microparticle, surfactant free SPION |
| P-039-003 | P39 | SP-339 | Polystyrene carboxyl functionalized SPION |
| P-041-001 | P41 | SP-341 | Carboxylic acid SPION |
| P-047-001 | P47 | SP-365 | Silica SPION |
| P-048-001 | P48 | SP-348 | Carboxylic acid, 150 nm SPION |
| P-053-001 | P53 | SP-353 | Amino surface microparticle, 0.4-0.6 µm SPION |
| P-056-001 | P56 | SP-356 | Silica amino functionalized microparticle, 0.1-0.39 µm SPION |
| P-063-001 | P63 | SP-363 | Jeffamine surface, 0.1-0.39 µm SPION |
| P-064-001 | P64 | SP-364 | Polystyrene microparticle, 2.0-2.9 µm SPION |
| P-065-001 | P65 | SP-365 | Silica SPION |
| P-069-001 | P69 | SP-369 | Carboxylated Original coating, 50 nm SPION |
| P-073-001 | P73 | SP-373 | Dextran based coating, 0.13 µm SPION |
| P-074-001 | P74 | SP-374 | Silica Silanol coated with lower acidity SPION |

A particle of the present disclosure may be synthesized, or a particle of the present disclosure may be purchased from a commercial vendor. For example, particles consistent with the present disclosure may be purchased from commercial vendors including Sigma-Aldrich, Life Technologies, Fisher Biosciences, nanoComposix, Nanopartz, Spherotech, and other commercial vendors. In some cases, a particle of the present disclosure may be purchased from a commercial vendor and further modified, coated, or functionalized.

An example of a particle type of the present disclosure may be a carboxylate (Citrate) superparamagnetic iron oxide nanoparticle (SPION), a phenol-formaldehyde coated SPION, a silica-coated SPION, a polystyrene coated SPION, a carboxylated poly(styrene-co-methacrylic acid) coated SPION, a N-(3-Trimethoxysilylpropyl)diethylenetriamine coated SPION, a poly(N-(3-(dimethylamino)propyl) methacrylamide) (PDMAPMA)-coated SPION, a 1,2,4,5-Benzenetetracarboxylic acid coated SPION, a poly(Vinylbenzyltrimethylammonium chloride) (PVBTMAC) coated SPION, a carboxylate, PAA coated SPION, a poly(oligo(ethylene glycol) methyl ether methacrylate) (POEGMA)-coated SPION, a carboxylate microparticle, a polystyrene carboxyl functionalized particle, a carboxylic acid coated particle, a silica particle, a carboxylic acid particle of about 150 nm in diameter, an amino surface microparticle of about 0.4-0.6 µm in diameter, a silica amino functionalized microparticle of about 0.1-0.39 µm in diameter, a Jeffamine surface particle of about 0.1-0.39 µm in diameter, a polystyrene microparticle of about 2.0-2.9 µm in diameter, a silica particle, a carboxylated particle with an original coating of about 50 nm in diameter, a particle coated with a dextran based coating of about 0.13 µm in diameter, or a silica silanol coated particle with low acidity.

A particle may be provided at a range of concentrations. A particle may comprise a concentration between 100 fM and 100 nM. A particle may comprise a concentration between 100 fM and 10 pM. A particle may comprise a concentration between 1 pM and 100 pM. A particle may comprise a concentration between 10 pM and 1 nM. A particle may comprise a concentration between 100 pM and 10 nM. A particle may comprise a concentration between 1 nM and 100 nM. A particle may be contacted to a biological sample at a ratio of volume ratios. A solution comprising a particle may be combined with a biological sample, at a volume ratio of greater than about 100:1, about 100:1, about 80:1, about 60:1, about 50:1, about 40:1, about 30:1, about 25:1, about 20:1, about 15:1, about 12:1, about 10:1, about 8:1, about 6:1, about 5:1, about 4:1, about 3:1, about 5:2, about 2:1, about 3:2, about 1:1, about 2:3, about 1:2, about 2:5, about 1:3, about 1:4, about 1:5, about 1:6, about 1:8, about 1:10, about 1:12, about 1:15, about 1:20, about 1:25, about 1:30, about 1:40, about 1:50, about 1:60, about 1:80, about 1:100, or less than about 1:100.

Particles that are consistent with the present disclosure can comprise a wide range of sizes. In some cases, a particle of the present disclosure may be a nanoparticle. In some cases, a nanoparticle of the present disclosure may be from about 10 nm to about 1000 nm in diameter. For example, the nanoparticles disclosed herein can be at least 10 nm, at least 100 nm, at least 200 nm, at least 300 nm, at least 400 nm, at least 500 nm, at least 600 nm, at least 700 nm, at least 800 nm, at least 900 nm, from 10 nm to 50 nm, from 50 nm to 100 nm, from 100 nm to 150 nm, from 150 nm to 200 nm, from 200 nm to 250 nm, from 250 nm to 300 nm, from 300 nm to 350 nm, from 350 nm to 400 nm, from 400 nm to 450 nm, from 450 nm to 500 nm, from 500 nm to 550 nm, from 550 nm to 600 nm, from 600 nm to 650 nm, from 650 nm to 700 nm, from 700 nm to 750 nm, from 750 nm to 800 nm, from 800 nm to 850 nm, from 850 nm to 900 nm, from 100 nm to 300 nm, from 150 nm to 350 nm, from 200 nm to 400 nm, from 250 nm to 450 nm, from 300 nm to 500 nm, from 350 nm to 550 nm, from 400 nm to 600 nm, from 450 nm to 650 nm, from 500 nm to 700 nm, from 550 nm to 750 nm, from 600 nm to 800 nm, from 650 nm to 850 nm, from 700 nm to 900 nm, or from 10 nm to 900 nm in diameter. In some cases, a nanoparticle may be less than 1000 nm in diameter.

A particle of the present disclosure may be a microparticle. A microparticle may be a particle that is from about 1 µm to about 1000 µm in diameter. For example, the microparticles disclosed here can be at least 1 µm, at least 10 µm, at least 100 µm, at least 200 µm, at least 300 µm, at least 400 µm, at least 500 µm, at least 600 µm, at least 700 µm, at least 800 µm, at least 900 µm, from 10 µm to 50 µm, from 50 µm to 100 µm, from 100 µm to 150 µm, from 150 µm to 200 µm, from 200 µm to 250 µm, from 250 µm to 300 µm, from 300 µm to 350 µm, from 350 µm to 400 µm, from 400 µm to 450 µm, from 450 µm to 500 µm, from 500 µm to 550 µm, from 550 µm to 600 µm, from 600 µm to 650 µm, from 650 µm to 700 µm, from 700 µm to 750 µm, from 750 µm to 800 µm, from 800 µm to 850 µm, from 850 µm to 900 µm, from 100 µm to 300 µm, from 150 µm to 350 µm, from 200 µm to 400 µm, from 250 µm to 450 µm, from 300 µm to 500 µm, from 350 µm to 550 µm, from 400 µm to 600 µm, from 450 µm to 650 µm, from 500 µm to 700 µm, from 550 µm to 750 µm, from 600 µm to 800 µm, from 650 µm to 850 µm, from 700 µm to 900 µm, or from 10 µm to 900 µm in diameter. In some cases, a microparticle may be less than 1000 µm in diameter.

The ratio between surface area and mass can affect a particle’s properties and biomolecule enrichment. For example, the number and types of biomolecules that a particle adsorbs from a solution may vary with the particle’s surface area to mass ratio. The particles disclosed herein can have surface area to mass ratios of 3 to 30 cm²/mg, 5 to 50 cm²/mg, 10 to 60 cm²/mg, 15 to 70 cm²/mg, 20 to 80 cm²/mg, 30 to 100 cm²/mg, 35 to 120 cm²/mg, 40 to 130 cm²/mg, 45 to 150 cm²/mg, 50 to 160 cm²/mg, 60 to 180 cm²/mg, 70 to 200 cm²/mg, 80 to 220 cm²/mg, 90 to 240 cm²/mg, 100 to 270 cm²/mg, 120 to 300 cm²/mg, 200 to 500 cm²/mg, 10 to 300 cm²/mg, 1 to 3000 cm²/mg, 20 to 150 cm²/mg, 25 to 120 cm²/mg, or from 40 to 85 cm²/mg. Small particles (e.g., with diameters of 50 nm or less) can have higher surface area to mass ratios than large particles (e.g., with diameters of 200 nm or more).. In some cases (e.g., for small particles), the particles can have surface area to mass ratios of 200 to 1000 cm²/mg, 500 to 2000 cm²/mg, 1000 to 4000 cm²/mg, 2000 to 8000 cm²/mg, or 4000 to 10000 cm²/mg. In some cases (e.g., for large particles), the particles can have surface area to mass ratios of 1 to 3 cm²/mg, 0.5 to 2 cm²/mg, 0.25 to 1.5 cm²/mg, or 0.1 to 1 cm²/mg.

In some cases, a plurality of particles (e.g., of a particle panel) of the compositions and methods described herein may comprise a range of surface area to mass ratios. In some cases, the range of surface area to mass ratios for a plurality of particles is less than 100 cm²/mg, 80 cm²/mg, 60 cm²/mg, 40 cm²/mg, 20 cm²/mg, 10 cm²/mg, 5 cm²/mg, or 2 cm²/mg. In some cases, the surface area to mass ratios for a plurality of particles varies by no more than 40%, 30%, 20%, 10%, 5%, 3%, 2%, or 1% between the particles in the plurality.

In some cases, a plurality of particles (e.g., in a particle panel) may have a wider range of surface area to mass ratios. In some cases, the range of surface area to mass ratios for a plurality of particles is greater than 100 cm²/mg, 150 cm²/mg, 200 cm²/mg, 250 cm²/mg, 300 cm²/mg, 400 cm²/mg, 500 cm²/mg, 800 cm²/mg, 1000 cm²/mg, 1200 cm²/mg, 1500 cm²/mg, 2000 cm²/mg, 3000 cm²/mg, 5000 cm²/mg, 7500 cm²/mg, 10000 cm²/mg, or more. In some cases, the surface area to mass ratios for a plurality of particles (e.g., within a panel) can vary by more than 100%, 200%, 300%, 400%, 500%, 1000%, 10000% or more. In some cases, the plurality of particles with a wide range of surface area to mass ratios comprises at least 2, 3, 4, 5, 6, 7, 8, 9, 10, 12, 15, 20, or more different types of particles.

A particle may comprise a wide array of physical properties. A physical property of a particle may include composition, size, surface charge, hydrophobicity, hydrophilicity, surface functionalization, surface topography, surface curvature, porosity, core material, shell material, shape, and any combination thereof.

A surface functionalization may comprise a polymerizable functional group, a positively or negatively charged functional group, a zwitterionic functional group, an acidic or basic functional group, a polar functional group, or any combination thereof. A surface functionalization may comprise carboxyl groups, hydroxyl groups, thiol groups, cyano groups, nitro groups, ammonium groups, alkyl groups, imidazolium groups, sulfonium groups, pyridinium groups, pyrrolidinium groups, phosphonium groups, aminopropyl groups, amine groups, boronic acid groups, N-succinimidyl ester groups, PEG groups, streptavidin, methyl ether groups, triethoxylpropylaminosilane groups, PCP groups, citrate groups, lipoic acid groups, BPEI groups, or any combination thereof. A particle from among the plurality of particles may be selected from the group consisting of: micelles, liposomes, iron oxide particles, silver particles, gold particles, palladium particles, quantum dots, platinum particles, titanium particles, silica particles, metal or inorganic oxide particles, synthetic polymer particles, copolymer particles, terpolymer particles, polymeric particles with metal cores, polymeric particles with metal oxide cores, polystyrene sulfonate particles, polyethylene oxide particles, polyoxyethylene glycol particles, polyethylene imine particles, polylactic acid particles, polycaprolactone particles, polyglycolic acid particles, poly(lactide-co-glycolide polymer particles, cellulose ether polymer particles, polyvinylpyrrolidone particles, polyvinyl acetate particles, polyvinylpyrrolidone-vinyl acetate copolymer particles, polyvinyl alcohol particles, acrylate particles, polyacrylic acid particles, crotonic acid copolymer particles, polyethlene phosphonate particles, polyalkylene particles, carboxy vinyl polymer particles, sodium alginate particles, carrageenan particles, xanthan gum particles, gum acacia particles, Arabic gum particles, guar gum particles, pullulan particles, agar particles, chitin particles, chitosan particles, pectin particles, karaya tum particles, locust bean gum particles, maltodextrin particles, amylose particles, corn starch particles, potato starch particles, rice starch particles, tapioca starch particles, pea starch particles, sweet potato starch particles, barley starch particles, wheat starch particles, hydroxypropylated high amylose starch particles, dextrin particles, levan particles, elsinan particles, gluten particles, collagen particles, whey protein isolate particles, casein particles, milk protein particles, soy protein particles, keratin particles, polyethylene particles, polycarbonate particles, polyanhydride particles, polyhydroxyacid particles, polypropylfumerate particles, polycaprolactone particles, polyamine particles, polyacetal particles, polyether particles, polyester particles, poly(orthoester) particles, polycyanoacrylate particles, polyurethane particles, polyphosphazene particles, polyacrylate particles, polymethacrylate particles, polycyanoacrylate particles, polyurea particles, polyamine particles, polystyrene particles, poly(lysine) particles, chitosan particles, dextran particles, poly(acrylamide) particles, derivatized poly(acrylamide) particles, gelatin particles, starch particles, chitosan particles, dextran particles, gelatin particles, starch particles, poly-β-amino-ester particles, poly(amido amine) particles, poly lactic-co-glycolic acid particles, polyanhydride particles, bioreducible polymer particles, and 2-(3-aminopropylamino)ethanol particles, and any combination thereof.

Particles of the present disclosure may differ by one or more physicochemical property. The one or more physicochemical property is selected from the group consisting of: composition, size, surface charge, hydrophobicity, hydrophilicity, roughness, density surface functionalization, surface topography, surface curvature, porosity, core material, shell material, shape, and any combination thereof. The surface functionalization may comprise a macromolecular functionalization, a small molecule functionalization, or any combination thereof. A small molecule functionalization may comprise an aminopropyl functionalization, amine functionalization, boronic acid functionalization, carboxylic acid functionalization, alkyl group functionalization, N-succinimidyl ester functionalization, monosaccharide functionalization, phosphate sugar functionalization, sulfurylated sugar functionalization, ethylene glycol functionalization, streptavidin functionalization, methyl ether functionalization, trimethoxysilylpropyl functionalization, silica functionalization, triethoxylpropylaminosilane functionalization, thiol functionalization, PCP functionalization, citrate functionalization, lipoic acid functionalization, ethyleneimine functionalization. A particle panel may comprise a plurality of particles with a plurality of small molecule functionalizations selected from the group consisting of silica functionalization, trimethoxysilylpropyl functionalization, dimethylamino propyl functionalization, phosphate sugar functionalization, amine functionalization, and carboxyl functionalization.

A small molecule functionalization may comprise a polar functional group. Non-limiting examples of polar functional groups comprise carboxyl group, a hydroxyl group, a thiol group, a cyano group, a nitro group, an ammonium group, an imidazolium group, a sulfonium group, a pyridinium group, a pyrrolidinium group, a phosphonium group or any combination thereof. In some embodiments, the functional group is an acidic functional group (e.g., sulfonic acid group, carboxyl group, and the like), a basic functional group (e.g., amino group, cyclic secondary amino group (such as pyrrolidyl group and piperidyl group), pyridyl group, imidazole group, guanidine group, etc.), a carbamoyl group, a hydroxyl group, an aldehyde group and the like.

A small molecule functionalization may comprise an ionic or ionizable functional group. Non-limiting examples of ionic or ionizable functional groups comprise an ammonium group, an imidazolium group, a sulfonium group, a pyridinium group, a pyrrolidinium group, a phosphonium group.

A small molecule functionalization may comprise a polymerizable functional group. Non-limiting examples of the polymerizable functional group include a vinyl group and a (meth)acrylic group. In some embodiments, the functional group is pyrrolidyl acrylate, acrylic acid, methacrylic acid, acrylamide, 2-(dimethylamino)ethyl methacrylate, hydroxyethyl methacrylate and the like.

A surface functionalization may comprise a charge. For example, a particle can be functionalized to carry a net neutral surface charge, a net positive surface charge, a net negative surface charge, or a zwitterionic surface. Surface charge can be a determinant of the types of biomolecules collected on a particle. Accordingly, optimizing a particle panel may comprise selecting particles with different surface charges, which may not only increase the number of different proteins collected on a particle panel, but also increase the likelihood of identifying a biological state of a sample. A particle panel may comprise a positively charged particle and a negatively charged particle. A particle panel may comprise a positively charged particle and a neutral particle. A particle panel may comprise a positively charged particle and a zwitterionic particle. A particle panel may comprise a neutral particle and a negatively charged particle. A particle panel may comprise a neutral particle and a zwitterionic particle. A particle panel may comprise a negative particle and a zwitterionic particle. A particle panel may comprise a positively charged particle, a negatively charged particle, and a neutral particle. A particle panel may comprise a positively charged particle, a negatively charged particle, and a zwitterionic particle. A particle panel may comprise a positively charged particle, a neutral particle, and a zwitterionic particle. A particle panel may comprise a negatively charged particle, a neutral particle, and a zwitterionic particle.

The present disclosure includes compositions (e.g., particle panels) and methods that comprise two or more particles differing in at least one physicochemical property. A composition or method of the present disclosure may comprise 3 to 6 particles differing in at least one physicochemical property. A composition or method of the present disclosure may comprise 4 to 8 particles differing in at least one physicochemical property. A composition or method of the present disclosure may comprise 4 to 10 particles differing in at least one physicochemical property. A composition or method of the present disclosure may comprise 5 to 12 particles differing in at least one physicochemical property. A composition or method of the present disclosure may comprise 6 to 14 particles differing in at least one physicochemical property. A composition or method of the present disclosure may comprise 8 to 15 particles differing in at least one physicochemical property. A composition or method of the present disclosure may comprise 10 to 20 particles differing in at least one physicochemical property. A composition or method of the present disclosure may comprise at least 2 distinct particle types, at least 3 distinct particle types, at least 4 distinct particle types, at least 5 distinct particle types, at least 6 distinct particle types, at least 7 distinct particle types, at least 8 distinct particle types, at least 9 distinct particle types, at least 10 distinct particle types, at least 11 distinct particle types, at least 12 distinct particle types, at least 13 distinct particle types, at least 14 distinct particle types, at least 15 distinct particle types, at least 20 distinct particle types, at least 25 particle types, or at least 30 distinct particle types.

A particle of the present disclosure may be contacted with a biological sample (e.g., a biofluid) to form a biomolecule corona. The particle and biomolecule corona may be separated from the biological sample, for example by centrifugation, magnetic separation, filtration, chromatographic separation, or gravitational separation. The particle types and biomolecule corona may be separated from the biological sample using a number of separation techniques. Non-limiting examples of separation techniques include comprises magnetic separation, charge-based separation, column-based separation, filtration, spin column-based separation, centrifugation, ultracentrifugation, density or gradient-based centrifugation, gravitational separation, or any combination thereof. Each of a plurality of particle types may be separated from a mixture of particles based on their physical (e.g., charge), chemical, or magnetic properties. Protein corona analysis may be performed on the separated particle and biomolecule corona. Protein corona analysis may comprise identifying one or more proteins in the biomolecule corona, for example by mass spectrometry. A single particle type (e.g., a particle of a type listed in TABLE 1) may be contacted to a biological sample. A plurality of particle types (e.g., a plurality of the particle types provided in TABLE 1) may be contacted to a biological sample. The plurality of particle types may be combined and contacted to the biological sample in a single sample volume. The plurality of particle types may be sequentially contacted to a biological sample and separated from the biological sample prior to contacting a subsequent particle type to the biological sample. Protein corona analysis of the biomolecule corona may compress the dynamic range of the analysis compared to a total protein analysis method.

The particles of the present disclosure may be used to serially interrogate a sample by incubating a first particle type with the sample to form a biomolecule corona on the first particle type, separating the first particle type, incubating a second particle type with the sample to form a biomolecule corona on the second particle type, separating the second particle type, and repeating the interrogating (by incubation with the sample) and the separating for any number of particle types. In some cases, the biomolecule corona on each particle type used for serial interrogation of a sample may be analyzed by protein corona analysis. The biomolecule content of the supernatant may be analyzed following serial interrogation with one or more particle types.

### Particle Panels

The present disclosure provides compositions and methods of use thereof for assaying a sample for proteins. Compositions described herein include particle panels comprising one or more than one distinct particle types. Particle panels described herein can vary in the number of particle types and the diversity of particle types in a single panel. For example, particles in a panel may vary based on size, polydispersity, shape and morphology, surface charge, surface chemistry and functionalization, and base material. Panels may be incubated with a sample to be analyzed for proteins and protein concentrations. Proteins in the sample adsorb to the surface of the different particle types in the particle panel to form a protein corona. The exact protein and the concentration of protein that adsorbs to a certain particle type in the particle panel may depend on the composition, size, and surface charge of said particle type. Thus, each particle type in a panel may have different protein coronas due to adsorbing a different set of proteins, different concentrations of a particular protein, or a combination thereof. Each particle type in a panel may have mutually exclusive protein coronas or may have overlapping protein coronas. Overlapping protein coronas can overlap in protein identity, in protein concentration, or both.

The present disclosure also provides methods for selecting a particle types for inclusion in a panel depending on the sample type. Particle types included in a panel may be a combination of particles that are optimized for removal of highly abundant proteins. Particle types also consistent for inclusion in a panel are those selected for adsorbing particular proteins of interest. The particles can be nanoparticles. The particles can be microparticles. The particles can be a combination of nanoparticles and microparticles.

A particle panel including any number of distinct particle types disclosed herein, enriches and identifies a single protein or protein group. In some cases, the single protein or protein group may comprise proteins having different post-translational modifications. For example, a first particle type in the particle panel may enrich a protein or protein group having a first post-translational modification, a second particle type in the particle panel may enrich the same protein or same protein group having a second post-translational modification, and a third particle type in the particle panel may enrich the same protein or same protein group lacking a post-translational modification. In some cases, the particle panel including any number of distinct particle types disclosed herein, enriches and identifies a single protein or protein group by binding different domains, sequences, or epitopes of the single protein or protein group. For example, a first particle type in the particle panel may enrich a protein or protein group by binding to a first domain of the protein or protein group, and a second particle type in the particle panel may enrich the same protein or same protein group by binding to a second domain of the protein or protein group.

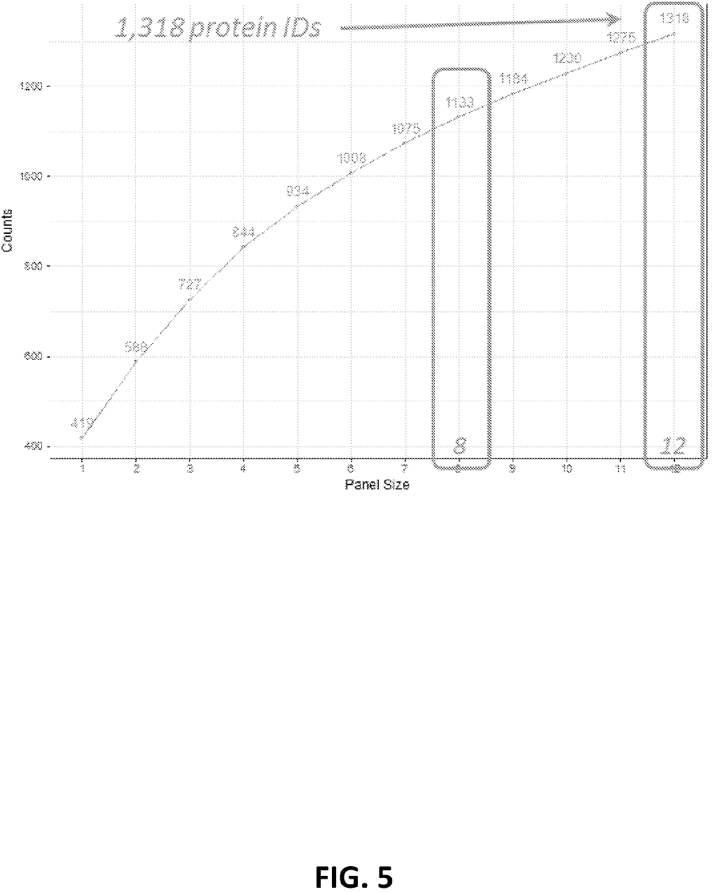

A particle panel can have more than one particle type. Increasing the number of particle types in a panel can be a method for increasing the number of proteins that can be identified in a given sample. An example of how increasing panel size may increase the number of identified proteins is shown in FIG. 5, in which a panel size of one particle type identified 419 different proteins, a panel size of two particle types identified 588 different proteins, a panel size of three particle types identified 727 different proteins, a panel size of four particle types identified 844 proteins, a panel size of five particle types identified 934 different proteins, a panel size of six particle types identified 1008 different proteins, a panel size of seven particle types identified 1075 different proteins, a panel size of eight particle types identified 1133 different proteins, a panel size of nine particle types identified 1184 different proteins, a panel size of 10 particle types identified 1230 different proteins, a panel size of 11 particle types identified 1275 different proteins, and a panel size of 12 particle types identified 1318 different proteins.

A particle panel may comprise a combination of particles with silica and polymer surfaces. For example, a particle panel may comprise a SPION coated with a thin layer of silica, a SPION coated with poly(dimethyl aminopropyl methacrylamide) (PDMAPMA), and a SPION coated with poly(ethylene glycol) (PEG). A particle panel consistent with the present disclosure could also comprise two or more particles selected from the group consisting of silica coated SPION, an N-(3-Trimethoxysilylpropyl) diethylenetriamine coated SPION, a PDMAPMA coated SPION, a carboxyl-functionalized polyacrylic acid coated SPION, an amino surface functionalized SPION, a polystyrene carboxyl functionalized SPION, a silica particle, and a dextran coated SPION. A particle panel consistent with the present disclosure may also comprise two or more particles selected from the group consisting of a surfactant free carboxylate microparticle, a carboxyl functionalized polystyrene particle, a silica coated particle, a silica particle, a dextran coated particle, an oleic acid coated particle, a boronated nanopowder coated particle, a PDMAPMA coated particle, a Poly(glycidyl methacrylate-benzylamine) coated particle, and a Poly(N-[3-(Dimethylamino)propyl]methacrylamide-co-[2-(methacryloyloxy)ethyl]dimethyl-(3-sulfopropyl)ammonium hydroxide, P(DMAPMA-co-SBMA) coated particle. A particle panel consistent with the present disclosure may comprise silica-coated particles, N-(3-Trimethoxysilylpropyl)diethylenetriamine coated particles, poly(N-(3-(dimethylamino)propyl) methacrylamide) (PDMAPMA)-coated particles, phosphate-sugar functionalized polystyrene particles, amine functionalized polystyrene particles, polystyrene carboxyl functionalized particles, ubiquitin functionalized polystyrene particles, dextran coated particles, or any combination thereof.

A particle panel consistent with the present disclosure may comprise a silica functionalized particle, an amine functionalized particle, a silicon alkoxide functionalized particle, a carboxylate functionalized particle, and a benzyl or phenyl functionalized particle. A particle panel consistent with the present disclosure may comprise a silica functionalized particle, an amine functionalized particle, a silicon alkoxide functionalized particle, a polystyrene functionalized particle, and a saccharide functionalized particle. A particle panel consistent with the present disclosure may comprise a silica functionalized particle, an N-(3-Trimethoxysilylpropyl)diethylenetriamine functionalized particle, a PDMAPMA functionalized particle, a dextran functionalized particle, and a polystyrene carboxyl functionalized particle. A particle panel consistent with the present disclosure may comprise 5 particles including a silica functionalized particle, an amine functionalized particle, a silicon alkoxide functionalized particle.

### Biomolecule Coronas

The present disclosure provides a variety of compositions, systems, and methods for collecting biomolecules on nanoparticles, microparticles, and other types of sensor elements such as polymer matrices, filters, rods, mesoporous materials, and extended surfaces. A particle may adsorb a plurality of biomolecules upon contact with a biological sample, thereby forming a biomolecule corona on the surfaces of the particles. The biomolecule corona may comprise proteins, lipids, nucleic acids, metabolites, saccharides, small molecules (e.g., sterols), and other biological species present in a sample. A biomolecule corona comprising proteins may also be referred to as a ‘protein corona’, and may refer to all constituents adsorbed to a particle (e.g., proteins, lipids, nucleic acids, and other biomolecules), or may refer only to proteins adsorbed to the particle.

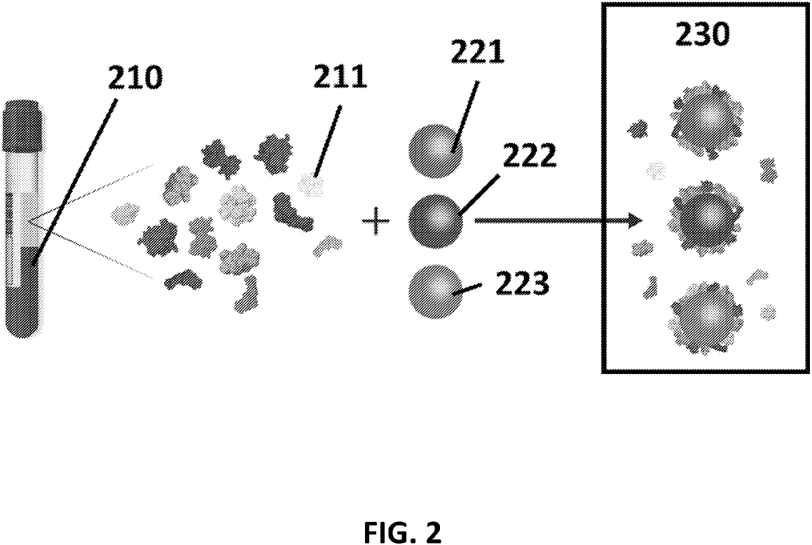

FIG. 2 provides a schematic overview of biomolecule formation, wherein a plurality of particles 221, 222, & 223 particles are contacted with a biological sample 210 comprising biomolecules molecules 211, and wherein each particle adsorbs a plurality of biomolecules from the biological sample to its surface 230. The different particles may be distinct particle types (depicted in the center of the figure, with the top, middle, and bottom spheres representing the three distinct particle types), such that each particle differs from the other particles by at least one physicochemical property. This difference in physicochemical properties can lead to the formation of different protein corona compositions on the particle surfaces.

The composition of the biomolecule corona may depend on a property of the particle. In many cases, the composition of the biomolecule corona is strongly dependent on the surface of the particle. Characteristics such as particle surface material (e.g., ceramic, polymer, metal, metal oxide, graphite, silicon dioxide, etc.), surface texture (rough, smooth, grooved, etc.), surface functionalization (e.g., carboxylate functionalized, amine functionalized, small molecule (e.g., saccharide) functionalized, etc.), shape, curvature, and size can each independently serve as major determinants for biomolecule corona composition. In addition to surface features, the particle core composition, particle density, and particle surface area to mass ratio may each influence biomolecule corona composition. For example, two particles comprising the same surfaces and different cores may form different biomolecule coronas upon contact with the same sample.

Biomolecule corona formation may also be influenced by sample composition. For example, a first sample condition (e.g., low salinity) might favor the solubility of a particular analyte (e.g., an isoform of Bone Morphogenic Protein 1 (BMP1)), and thereby disfavor its binding in a biomolecule corona, while a second sample condition (e.g., high salinity) may diminish the solubility of the analyte, thereby driving its incorporation into a biomolecule corona.

Biomolecule corona composition may also depend on molecular level interactions between the biomolecules, themselves. An energetically favorable interaction between two biomolecules may promote their co-incorporation into a biomolecule corona. For example, if a first protein adsorbed to a particle comprises an affinity for a second protein in solution, the first protein may bind to a portion of the second protein, thereby driving its binding to the particle or to other proteins of the biomolecule corona of the particle. Analogously, a first biomolecule disposed within a biomolecule corona may comprise an energetically unfavorable interaction with a second biomolecule in a biological sample, thereby disfavoring its incorporation into a biomolecule corona. In part owing to these inter-biomolecule dependencies, biomolecule coronas provide sensitive platforms for directly and indirectly sensing biomolecules from a biological sample.

### Biomolecule Analysis Methods

The present disclosure provides a range of methods for analyzing biomolecules. A biomolecule may be analyzed prior to its collection on a particle. A biomolecule may be analyzed as it is disposed within a biomolecule corona. A biomolecule may be subjected to analysis after it is released from a particle. For example, a biomolecule corona or a portion of a biomolecule corona may be separated from a particle and analyzed. A biomolecule corona or a portion of a biomolecule corona may be digested as it is disposed on a particle, and subjected to further analysis. A biomolecule may be analyzed on a first particle at a first time and on a second particle at a second time.

A biomolecule may be analyzed with an affinity reagent. An affinity reagent may be contacted to a biomolecule corona. An affinity may be contacted to eluent or digestion products of a biomolecule corona. In addition to or in place of affinity reagent analysis, a biomolecule (e.g., a biomolecule of a biomolecule corona) may be analyzed spectroscopically, such as with circular dichroism, absorbance spectroscopy, Raman spectroscopy, resonance Raman spectroscopy, infrared spectroscopy, mass spectrometry, inductively-coupled plasma mass spectrometry (e.g., for compositional analysis), electrochemical analysis, nuclear magnetic resonance spectroscopy, electron paramagnetic resonance spectroscopy, diffraction (e.g., X-ray, electron, or ion), electrophoresis, histological analysis, or any combination thereof. A biopolymer (e.g., a biopolymer of a biomolecule corona) may be sequenced, for example with mass spectrometry, nuclear magnetic resonance spectroscopy, nanopore sequencing (e.g., porin translocation), Edman degradation, fluorosequencing, next-generation nucleic acid sequencing, or any combination thereof.

Particles and methods of use thereof disclosed herein can bind a large number of unique biomolecules (e.g., distinct protein types) present in a biological sample (e.g., a biofluid). For example, a particle disclosed herein can be incubated with a biological sample to form a protein corona comprising at least 5 unique proteins, at least 10 unique proteins, at least 15 unique proteins, at least 20 unique proteins, at least 25 unique proteins, at least 30 unique proteins, at least 40 unique proteins, at least 50 unique proteins, at least 60 unique proteins, at least 80 unique proteins, 100 unique proteins, at least 120 unique proteins, at least 140 unique proteins, at least 160 unique proteins, at least 180 unique proteins, at least 200 unique proteins, at least 220 unique proteins, at least 240 unique proteins, at least 260 unique proteins, at least 280 unique proteins, at least 300 unique proteins, at least 320 unique proteins, at least 340 unique proteins, at least 360 unique proteins, at least 380 unique proteins, at least 400 unique proteins, at least 420 unique proteins, at least 440 unique proteins, at least 460 unique proteins, at least 480 unique proteins, at least 500 unique proteins, at least 520 unique proteins, at least 540 unique proteins, at least 560 unique proteins, at least 580 unique proteins, at least 600 unique proteins, at least 620 unique proteins, at least 640 unique proteins, at least 660 unique proteins, at least 680 unique proteins, at least 700 unique proteins, at least 720 unique proteins, at least 740 unique proteins, at least 760 unique proteins, at least 780 unique proteins, at least 800 unique proteins, at least 820 unique proteins, at least 840 unique proteins, at least 860 unique proteins, at least 880 unique proteins, at least 900 unique proteins, at least 920 unique proteins, at least 940 unique proteins, at least 960 unique proteins, at least 980 unique proteins, at least 1000 unique proteins, at least 1100 unique proteins, at least 1200 unique proteins, at least 1300 unique proteins, at least 1400 unique proteins, at least 1500 unique proteins, at least 1600 unique proteins, at least 1800 unique proteins, at least 2000 unique proteins, from 100 to 2000 unique proteins, from 150 to 1500 unique proteins, from 200 to 1200 unique proteins, from 250 to 850 unique proteins, from 300 to 800 unique proteins, from 350 to 750 unique proteins, from 400 to 700 unique proteins, from 450 to 650 unique proteins, from 500 to 600 unique proteins, from 200 to 250 unique proteins, from 250 to 300 unique proteins, from 300 to 350 unique proteins, from 350 to 400 unique proteins, from 400 to 450 unique proteins, from 450 to 500 unique proteins, from 500 to 550 unique proteins, from 550 to 600 unique proteins, from 600 to 650 unique proteins, from 650 to 700 unique proteins, from 700 to 750 unique proteins, from 750 to 800 unique proteins, from 800 to 850 unique proteins, from 850 to 900 unique proteins, from 900 to 950 unique proteins, from 950 to 1000 unique proteins, or over 1000 unique proteins. In some cases, several different types of particles can be used, separately or in combination, to identify large numbers of proteins in a particular biological sample. In other words, particles can be multiplexed in order to bind and identify large numbers of proteins in a biological sample. Protein corona analysis may compress the dynamic range of the analysis compared to a protein analysis of the original sample.

The particle panels disclosed herein can be used to identify the number of distinct proteins disclosed herein, and/or any of the specific proteins disclosed herein, over a wide dynamic range. As used herein, a dynamic range may denote a log10 value of a ratio of the highest and lowest abundance species of a specified type. Enriching or assaying species over a dynamic range may refer to the abundances of those species in the sample from which they are assayed or derived. For example, the particle panels disclosed herein comprising distinct particle types, can enrich for proteins in a sample, which can be identified using the Proteograph workflow, over the entire dynamic range at which proteins are present in a sample (e.g., a plasma sample). In some cases, a particle panel including any number of distinct particle types disclosed herein, enriches and identifies proteins over a dynamic range of at least 2. In some cases, a particle panel including any number of distinct particle types disclosed herein, enriches and identifies proteins over a dynamic range of at least 3. In some cases, a particle panel including any number of distinct particle types disclosed herein, enriches and identifies proteins over a dynamic range of at least 4. In some cases, a particle panel including any number of distinct particle types disclosed herein, enriches and identifies proteins over a dynamic range of at least 5. In some cases, a particle panel including any number of distinct particle types disclosed herein, enriches and identifies proteins over a dynamic range of at least 6. In some cases, a particle panel including any number of distinct particle types disclosed herein, enriches and identifies proteins over a dynamic range of at least 7. In some cases, a particle panel including any number of distinct particle types disclosed herein, enriches and identifies proteins over a dynamic range of at least 8. In some cases, a particle panel including any number of distinct particle types disclosed herein, enriches and identifies proteins over a dynamic range of at least 9. In some cases, a particle panel including any number of distinct particle types disclosed herein, enriches and identifies proteins over a dynamic range of at least 10. In some cases, a particle panel including any number of distinct particle types disclosed herein, enriches and identifies proteins over a dynamic range of at least 11. In some cases, a particle panel including any number of distinct particle types disclosed herein, enriches and identifies proteins over a dynamic range of at least 12. In some cases, a particle panel including any number of distinct particle types disclosed herein, enriches and identifies proteins over a dynamic range of at least 13. In some cases, a particle panel including any number of distinct particle types disclosed herein, enriches and identifies proteins over a dynamic range of at least 14. In some cases, a particle panel including any number of distinct particle types disclosed herein, enriches and identifies proteins over a dynamic range of at least 15. In some cases, a particle panel including any number of distinct particle types disclosed herein, enriches and identifies proteins over a dynamic range of at least 20. In some cases, a particle panel including any number of distinct particle types disclosed herein, enriches and identifies proteins over a dynamic range of from 2 to 100. In some cases, a particle panel including any number of distinct particle types disclosed herein, enriches and identifies proteins over a dynamic range of from 2 to 20. In some cases, a particle panel including any number of distinct particle types disclosed herein, enriches and identifies proteins over a dynamic range of from 2 to 10. In some cases, a particle panel including any number of distinct particle types disclosed herein, enriches and identifies proteins over a dynamic range of from 2 to 5. In some cases, a particle panel including any number of distinct particle types disclosed herein, enriches and identifies proteins over a dynamic range of from 5 to 10.

The numbers and types of biomolecules (e.g., proteins) collected in a biomolecule corona may depend on the amount of time a particle is incubated with a sample. In many cases, biomolecule corona formation will comprise a time dependence, such that different sets of biomolecules collect on a particle at different rates. Further complicating this process, a biomolecule can comprise a time-dependent adsorption or desorption profile. For example, a biomolecule may rapidly collect on a particle during a first phase of biomolecule corona formation, and subsequently slowly desorb from the particle as other biomolecules bind. Accordingly, the length of time over which a particle is contacted to a sample can influence the mass and composition of a resulting biomolecule corona. An assay may generate a biomolecule corona in less than 2 hours. An assay may generate a biomolecule corona in less than 1.5 hours. An assay may generate a biomolecule corona in less than 1 hour. An assay may generate a biomolecule corona in less than 30 minutes. An assay may generate a biomolecule corona in less than 20 minutes. An assay may generate a biomolecule corona in less than 15 minutes. An assay may generate a biomolecule corona in less than 12 minutes. An assay may generate a biomolecule corona in less than 10 minutes. An assay may comprise incubating a particle with a sample for at least 10 minutes to generate a biomolecule corona. An assay may comprise incubating a particle with a sample for at least 12 minutes to generate a biomolecule corona. An assay may comprise incubating a particle with a sample for at least 15 minutes to generate a biomolecule corona. An assay may comprise incubating a particle with a sample for at least 20 minutes to generate a biomolecule corona. An assay may comprise incubating a particle with a sample for at least 30 minutes to generate a biomolecule corona. An assay may comprise incubating a particle with a sample for at least 45 minutes to generate a biomolecule corona. An assay may comprise incubating a particle with a sample for at least 60 minutes to generate a biomolecule corona. An assay may comprise incubating a particle with a sample for at least 90 minutes to generate a biomolecule corona. An assay may comprise incubating a particle with a sample for at least 120 minutes to generate a biomolecule corona. A biomolecule corona may comprise at least 10⁻¹¹ mg of biomolecules per square millimeter (mm²) of particle surface area. A biomolecule corona may comprise at least 5×10⁻¹¹ mg of biomolecules per square millimeter (mm²) of particle surface area. A biomolecule corona may comprise at least 10⁻¹⁰ mg of biomolecules per square millimeter (mm²) of particle surface area. A biomolecule corona may comprise at least 5×10⁻¹⁰ mg of biomolecules per square millimeter (mm²) of particle surface area. A biomolecule corona may comprise at least 10⁻⁹ mg of biomolecules per square millimeter (mm²) of particle surface area. A biomolecule corona may comprise at least 5×10⁻⁹ mg of biomolecules per square millimeter (mm²) of particle surface area. A biomolecule corona may comprise at least 10⁻⁸ mg of biomolecules per square millimeter (mm²) of particle surface area. A biomolecule corona may comprise at least of biomolecules per square millimeter (mm²) of particle surface area. A biomolecule corona may comprise at least 10⁻⁷ mg of biomolecules per square millimeter (mm²) of particle surface area. A biomolecule corona may comprise at least 10⁻¹¹ mg of proteins per square millimeter (mm²) of particle surface area. A biomolecule corona may comprise at least 5×10⁻¹¹ mg of proteins per square millimeter (mm²) of particle surface area. A biomolecule corona may comprise at least 10⁻¹⁰ mg of proteins per square millimeter (mm²) of particle surface area. A biomolecule corona may comprise at least 5×10⁻¹⁰ mg of proteins per square millimeter (mm²) of particle surface area. A biomolecule corona may comprise at least 10⁻⁹ mg of proteins per square millimeter (mm²) of particle surface area. A biomolecule corona may comprise at least 5×10⁻⁹ mg of proteins per square millimeter (mm²) of particle surface area. A biomolecule corona may comprise at least 10⁻⁸ mg of proteins per square millimeter (mm²) of particle surface area. A biomolecule corona may comprise at least 5×10⁻⁸ mg of proteins per square millimeter (mm²) of particle surface area. A biomolecule corona may comprise at least 10⁻⁷ mg of proteins per square millimeter (mm²) of particle surface area. A biomolecule corona may comprise an expanded or compressed dynamic range relative to a sample. For example, a biomolecule corona may collect proteins spanning 7 orders of magnitude in concentration in a sample over an abundance range spanning 4 orders of magnitude, thereby compressing the dynamic range of the collected proteins.

Biomolecules collected on a particle may be subjected to further analysis. A method may comprise generating a biomolecule corona, and subjecting the biomolecule corona or biomolecules derived from the biomolecule corona to affinity reagent based analysis, mass spectrometric analysis, circular dichroism, absorbance spectroscopy, Raman spectroscopy, resonance Raman spectroscopy, infrared spectroscopy, inductively-coupled plasma mass spectrometry (e.g., for compositional analysis), electrochemical analysis, nuclear magnetic resonance spectroscopy, electron paramagnetic resonance spectroscopy, diffraction (e.g., X-ray, electron, or ion), electrophoresis, histological analysis, or any combination thereof. The collected biomolecule corona or the collected subset of biomolecules from the biomolecule corona may be purified or fractionated (e.g., by a chromatographic method) prior to analysis, subsequent to analysis, or in place of analysis.

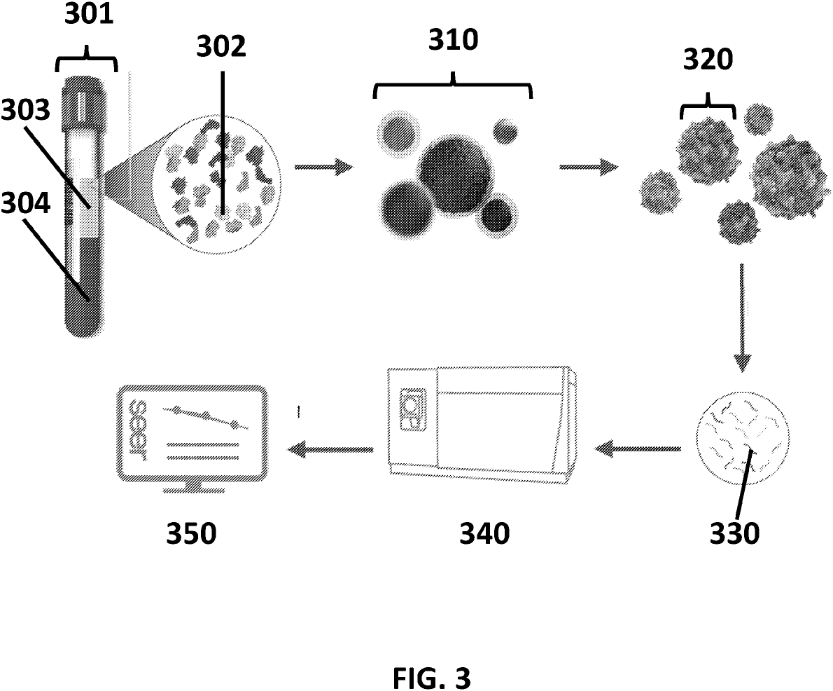

FIG. 3 provides an example of a particle-based biomolecule corona (e.g., protein corona) assay consistent with the present disclosure. A biological sample (e.g., human plasma) 301 comprising a plurality of biomolecules 302 may be contacted to a plurality of particles 310. The sample may be treated, diluted, or split into a plurality of fractions 303 and 304 prior to analysis. For example, a whole blood sample may be fractionated into plasma and erythrocyte portions. Upon contact with the particles, a subset or the entirety of the plurality of biomolecules may adsorb to the particles, thereby forming biomolecule coronas 320 bound to the surfaces of the particles. Unbound biomolecules may be separated from the biomolecule coronas (e.g., through wash steps). The biomolecule coronas, or subsets thereof, may be collected from the particles. Alternatively, biomolecules of the biomolecule coronas may be fragmented or chemically treated while bound to the particles. In some assays, biomolecules (e.g., proteins) are fragmented (e.g., digested) while disposed in the biomolecule coronas to yield biomolecule (e.g., peptide) fragments 330. Biomolecules (or their chemically treated or fragmented derivatives) may be analyzed 340, for example by mass spectrometry, to yield data 350 representative of biomolecules 302 from the biological sample 301. The data may be analyzed to identify a biological state of the biological sample.

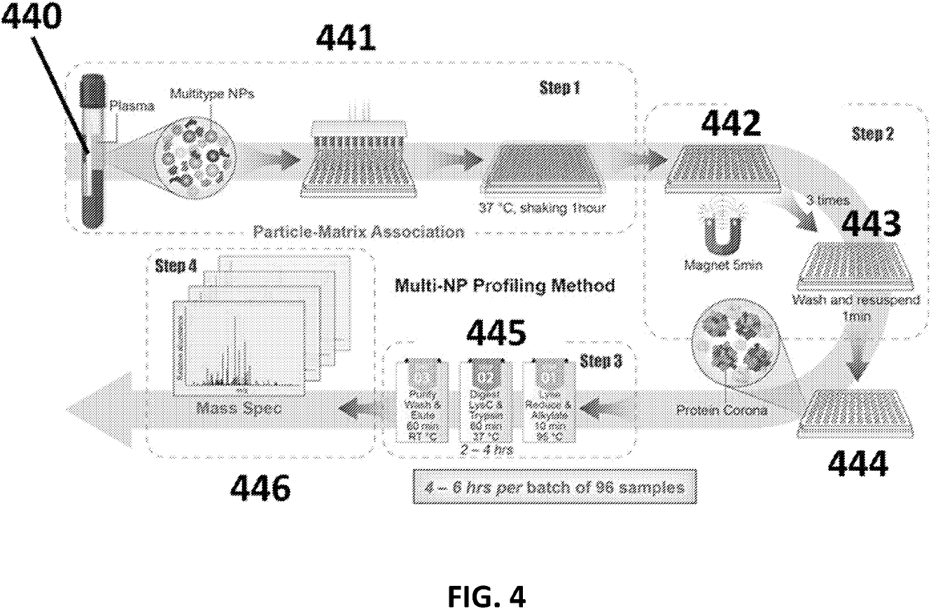

FIG. 4 illustrates an example of a biomolecule corona (e.g., protein corona) analysis workflow consistent with the present disclosure which includes: particle incubation with a biological sample 440 (e.g., plasma), thereby adsorbing biomolecules from the plasma sample to the particles to form biomolecule coronas; partitioning 441 of the particle-plasma sample mixture into a plurality of wells on a 96 well plate; particle collection 442 (e.g., with a magnet); a wash step or plurality of wash steps 443 to remove analytes not adsorbed to the particles; 444 resuspension of the particles and the biomolecules adsorbed thereto; optionally, biomolecule corona digestion or chemical treatment 445 (e.g., protein reduction and digestion); and analysis of the biomolecule coronas or of biomolecules derived therefrom 446 (e.g., by liquid chromatography-mass spectrometry (LC-MS) analysis). While this example provides parallel analyses across 96 well plate wells, a method may comprise a single sample volume or a plurality of sample volumes ranging from two to hundreds of thousands of sample volumes. Furthermore, while this example provides contacting a sample with particles prior to partitioning, a method may alternatively comprise partitioning a sample (e.g., into separate wells of a well plate) prior to contacting with particles. In some cases, sample may be added to partitions comprising particles. For example, a well plate may be provided with particles, buffer, and reagents in dry form, such that a method of use may comprise adding solution to the wells to resuspend the particles and dissolve the buffer and reagents, and then adding sample to the wells.

Protein corona analysis may comprise an automated component. For example, an automated instrument may contact a sample with a particle or particle panel, identify proteins on the particle or particle panel (e.g., digest the proteins on the particle or particle panel and perform mass spectrometric analysis), and generate data for identifying a specific biomolecule or a biological state of a sample. The automated instrument may divide a sample into a plurality of volumes, and perform analysis on each volume. The automated instrument may analyze multiple separate samples, for example by disposing multiple samples within multiple wells in a well plate, and performing parallel analysis on each sample.

The particle panels disclosed herein can be used to identifying a number of proteins, peptides, protein groups, or protein classes using a protein analysis workflow described herein (e.g., a protein corona analysis workflow). Protein corona analysis may comprise contacting a sample to distinct particle types (e.g., a particle panel), forming biomolecule corona on the distinct particle types, and identifying the biomolecules in the biomolecule corona (e.g., by mass spectrometry). Feature intensities, as disclosed herein, refers to the intensity of a discrete spike (“feature”) seen on a plot of mass to charge ratio versus intensity from a mass spectrometry run of a sample. These features can correspond to variably ionized fragments of peptides and/or proteins. Using the data analysis methods described herein, feature intensities can be sorted into protein groups. Protein groups refer to two or more proteins that are identified by a shared peptide sequence. Alternatively, a protein group can refer to one protein that is identified using a unique identifying sequence. For example, if in a sample, a peptide sequence is assayed that is shared between two proteins (Protein 1: XYZZX and Protein 2: XYZYZ), a protein group could be the “XYZ protein group” having two members (protein 1 and protein 2). Alternatively, if the peptide sequence is unique to a single protein (Protein 1), a protein group could be the “ZZX” protein group having one member (Protein 1). Each protein group can be supported by more than one peptide sequence. Protein detected or identified according to the instant disclosure can refer to a distinct protein detected in the sample (e.g., distinct relative other proteins detected using mass spectrometry). Thus, analysis of proteins present in distinct coronas corresponding to the distinct particle types in a particle panel yields a high number of feature intensities. This number decreases as feature intensities are processed into distinct peptides, further decreases as distinct peptides are processed into distinct proteins, and further decreases as peptides are grouped into protein groups (two or more proteins that share a distinct peptide sequence).

The methods disclosed herein include isolating one or more particle types from a sample or from more than one sample (e.g., a biological sample or a serially interrogated sample). The particle types can be rapidly isolated or separated from the sample using a magnet. Moreover, multiple samples that are spatially isolated can be processed in parallel. Thus, the methods disclosed herein provide for isolating or separating a particle type from unbound protein in a sample. A particle type may be separated by a variety of means, including but not limited to magnetic separation, centrifugation, filtration, or gravitational separation. Particle panels may be incubated with a plurality of spatially isolated samples, wherein each spatially isolated sample is in a well in a well plate (e.g., a 96-well plate). After incubation, the particle types in each of the wells of the well plate can be separated from unbound protein present in the spatially isolated samples by placing the entire plate on a magnet. This simultaneously pulls down the superparamagnetic particles in the particle panel. The supernatant in each sample can be removed to remove the unbound protein. These steps (incubate, pull down) can be repeated to effectively wash the particles, thus removing residual background unbound protein that may be present in a sample. This is one example, but one of skill in the art could envision numerous other scenarios in which superparamagnetic particles are rapidly isolated from one or more than one spatially isolated samples at the same time.

The methods and compositions of the present disclosure provide identification and measurement of particular proteins in the biological samples by processing of the proteomic data via digestion of coronas formed on the surface of particles. Examples of proteins that can be identified and measured include highly abundant proteins, proteins of medium abundance, and low-abundance proteins. A low abundance protein may be present in a sample at concentrations at or below about 10 ng/mL. A high abundance protein may be present in a sample at concentrations at or above about 10 µg/mL. A high abundance protein may be present in a sample at concentrations at or above about 1 µM. A high abundance protein may comprise at least 1%, at least 0.1%, or at least 0.05% of the protein mass of a sample. A protein of moderate abundance may be present in a sample at concentrations between about 10 ng/mL and about 10 µg/mL. Examples of proteins that are highly abundant in human plasma include albumin, IgG, and the top 14 proteins in abundance that contribute 95% of the analyte mass in plasma. Additionally, any proteins that may be purified using a conventional depletion column may be directly detected in a sample using the particle panels disclosed herein. Examples of proteins may be any protein listed in published databases such as Keshishian et al. (Mol Cell Proteomics. 2015 Sep;14(9):2375-93. doi: 10.1074/mcp.M114.046813. Epub 2015 Feb 27.), Farr et al. (J Proteome Res. 2014 Jan 3;13(1):60-75. doi: 10.1021/pr4010037. Epub 2013 Dec 6.), or Pernemalm et al. (Expert Rev Proteomics. 2014 Aug;11(4):431-48. doi: 10.1586/14789450.2014.901157. Epub 2014 Mar 24.).

The methods and compositions disclosed herein may also elucidate protein classes or interactions of the protein classes. A protein class may comprise a set of proteins that share a common function (e.g., amine oxidases or proteins involved in angiogenesis); proteins that share common physiological, cellular, or subcellular localization (e.g., peroxisomal proteins or membrane proteins); proteins that share a common cofactor (e.g., heme or flavin proteins); proteins that correspond to a particular biological state (e.g., hypoxia related proteins); proteins containing a particular structural motif (e.g., a cupin fold); or proteins bearing a post-translational modification (e.g., ubiquitinated or citrullinated proteins). A protein class may contain at least 2 proteins, 5 proteins, 10 proteins, 20 proteins, 40 proteins, 60 proteins, 80 proteins, 100 proteins, 150 proteins, 200 proteins, or more.

The proteomic data of the biological sample can be identified, measured, and quantified using a number of different analytical techniques. For example, proteomic data can be generated using SDS-PAGE or any gel-based separation technique. Peptides and proteins can also be identified, measured, and quantified using an immunoassay, such as ELISA. Alternatively, proteomic data can be identified, measured, and quantified using mass spectrometry, high performance liquid chromatography, LC-MS/MS, Edman Degradation, immunoaffinity techniques, methods disclosed in EP3548652, WO2019083856, WO2019133892, each of which is incorporated herein by reference in its entirety, and other protein separation techniques.

An assay may comprise protein collection of particles, protein digestion, and mass spectrometric analysis (e.g., MS, LC-MS, LC-MS/MS). The digestion may comprise chemical digestion, such as by cyanogen bromide or 2-Nitro-5-thiocyanatobenzoic acid (NTCB). The digestion may comprise enzymatic digestion, such as by trypsin or pepsin. The digestion may comprise enzymatic digestion by a plurality of proteases. The digestion may comprise a protease selected from among the group consisting of trypsin, chymotrypsin, Glu C, Lys C, elastase, subtilisin, proteinase K, thrombin, factor X, Arg C, papaine, Asp N, thermolysine, pepsin, aspartyl protease, cathepsin D, zinc mealloprotease, glycoprotein endopeptidase, proline, aminopeptidase, prenyl protease, caspase, kex2 endoprotease, or any combination thereof. The digestion may cleave peptides at random positions. The digestion may cleave peptides at a specific position (e.g., at methionines) or sequence (e.g., glutamate-histidine-glutamate). The digestion may enable similar proteins to be distinguished. For example, an assay may resolve 8 distinct proteins as a single protein group with a first digestion method, and as 8 separate proteins with distinct signals with a second digestion method. The digestion may generate an average peptide fragment length of 8 to 15 amino acids. The digestion may generate an average peptide fragment length of 12 to 18 amino acids. The digestion may generate an average peptide fragment length of 15 to 25 amino acids. The digestion may generate an average peptide fragment length of 20 to 30 amino acids. The digestion may generate an average peptide fragment length of 30 to 50 amino acids.

An assay may rapidly generate and analyze proteomic data. Beginning with an input biological sample (e.g., a buccal or nasal smear, plasma, or tissue), an assay of the present disclosure may generate and analyze proteomic data in less than 7 hours. Beginning with an input biological sample, an assay of the present disclosure may generate and analyze proteomic data in 5-7 hours. Beginning with an input biological sample, an assay of the present disclosure may generate and analyze proteomic data in less than 5 hours. Beginning with an input biological sample, an assay of the present disclosure may generate and analyze proteomic data in 3-5 hours. Beginning with an input biological sample, an assay of the present disclosure may generate and analyze proteomic data in 2-4 hours. Beginning with an input biological sample, an assay of the present disclosure may generate and analyze proteomic data in 2-3 hours. Beginning with an input biological sample, an assay of the present disclosure may generate and analyze proteomic data in less than 3 hours. Beginning with an input biological sample, an assay of the present disclosure may generate and analyze proteomic data in less than 2 hours. The analyzing may comprise identifying a protein group. The analyzing may comprise identifying a protein class. The analyzing may comprise quantifying an abundance of a biomolecule, a peptide, a protein, protein group, or a protein class. The analyzing may comprise identifying a ratio of abundances of two biomolecules, peptides, proteins, protein groups, or protein classes. The analyzing may comprise identifying a biological state.

### Dynamic Range

The biomolecule corona analysis methods described herein may comprise assaying biomolecules in a sample of the present disclosure across a wide dynamic range. The dynamic range of biomolecules assayed in a sample may be a range of measured signals of biomolecule abundances as measured by an assay method (e.g., mass spectrometry, chromatography, gel electrophoresis, spectroscopy, or immunoassays) for the biomolecules contained within a sample. For example, an assay capable of detecting proteins across a wide dynamic range may be capable of detecting proteins of very low abundance to proteins of very high abundance. The dynamic range of an assay may be directly related to the slope of assay signal intensity as a function of biomolecule abundance. For example, an assay with a low dynamic range may have a low (but positive) slope of the assay signal intensity as a function of biomolecule abundance, e.g., the ratio of the signal detected for a high abundance biomolecule to the ratio of the signal detected for a low abundance biomolecule may be lower for an assay with a low dynamic range than an assay with a high dynamic range. In specific cases, dynamic range may refer to the dynamic range of proteins within a sample or assaying method.

The biomolecule corona analysis methods described herein may compress the dynamic range of an assay. The dynamic range of an assay may be compressed relative to another assay if the slope of the assay signal intensity as a function of biomolecule abundance is lower than that of the other assay. For example, a plasma sample assayed using protein corona analysis with affinity reagents may have a compressed dynamic range compared to a plasma sample assayed using affinity reagents alone, directly on the sample or compared to provided abundance values for plasma proteins in databases (e.g., the database provided in Keshishian et al., Mol. Cell Proteomics 14, 2375-2393 (2015), also referred to herein as the “Carr database”).

The compressed dynamic range may enable the detection of lower abundance biomolecules or a greater number of low abundance biomolecules than would be possible solely with probes or conventional detection methods. For example, an affinity reagent comprising 6-orders of magnitude greater affinity for interleukin-10 than for serum albumin may exhibit negligible interleukin-10 binding in a plasma sample comprising about 10-orders of magnitude greater albumin than interleukin-10, but exhibit measurable interleukin-10 binding on a biomolecule corona comprising 6-orders of magnitude greater albumin than interleukin-10. As a particle may enrich a subset of biomolecules from a sample, a particle may enhance the detection capabilities of a probe to include a wide range of low abundance and low probe-affinity biomolecules.

In some embodiments, the dynamic range of a proteomic analysis assay may be the ratio of the signal produced by highest abundance proteins (e.g., the highest 10% of proteins by abundance) to the signal produced by the lowest abundance proteins (e.g., the lowest 10% of proteins by abundance). Compressing the dynamic range of a proteomic analysis may comprise decreasing the ratio of the signal produced by the highest abundance proteins to the signal produced by the lowest abundance proteins for a first proteomic analysis assay relative to that of a second proteomic analysis assay. The protein corona analysis assays disclosed herein may compress the dynamic range relative to the dynamic range of a total protein analysis method (e.g., mass spectrometry, gel electrophoresis, or liquid chromatography).

Provided herein are several methods for compressing the dynamic range of a biomolecular analysis assay to facilitate the detection of low abundance biomolecules relative to high abundance biomolecules. For example, a particle type of the present disclosure can be used to serially interrogate a sample. Upon incubation of the particle type in the sample, a biomolecule corona comprising forms on the surface of the particle type. If biomolecules are directly detected in the sample without the use of said particle types, for example by direct mass spectrometric analysis of the sample, the dynamic range may span a wider range of concentrations, or more orders of magnitude, than if the biomolecules are directed on the surface of the particle type. Thus, using the particle types disclosed herein may be used to compress the dynamic range of biomolecules in a sample. Without being limited by theory, this effect may be observed due to more capture of higher affinity, lower abundance biomolecules in the biomolecule corona of the particle type and less capture of lower affinity, higher abundance biomolecules in the biomolecule corona of the particle type.

A dynamic range of a proteomic assay may be the slope of a plot of a protein signal measured by the proteomic analysis assay as a function of total abundance of the protein in the sample. Compressing the dynamic range may comprise decreasing the slope of the plot of a protein signal measured by a proteomic analysis assay as a function of total abundance of the protein in the sample relative to the slope of the plot of a protein signal measured by a second proteomic analysis assay as a function of total abundance of the protein in the sample. The protein corona analysis assays disclosed herein may compress the dynamic range relative to the dynamic range of a total protein analysis method (e.g., mass spectrometry, gel electrophoresis, or liquid chromatography).

### Affinity Reagents and Probes

Disclosed herein are compositions of probes and affinity reagents, as well as methods of use thereof for rapid identification of proteins in a biological sample. The term ‘affinity reagent’ may refer to a molecule or complex of molecules (e.g., a light chain variable region and a heavy chain variable region of an antibody fragment antigen-binding (Fab) domain) that binds to a specific target. The target may be a molecule, a portion of a molecule (e.g., a site on the surface of a protein), a supramolecular structure (e.g., chromatin), an ion (e.g., Cu²⁺ or SO₄²⁻), or a material. An affinity reagent may bind to more than one target. An affinity reagent may have different binding affinities for different targets. An affinity reagent may be capable of simultaneously binding to multiple targets.

As used herein, the term ‘probe’ may refer to a molecule, complex, structure, or material comprising an affinity reagent. A probe may comprise a plurality of affinity reagents with identical or dissimilar analyte affinities. For example, a probe may comprise multiple scFv targeting different epitopes. A probe may comprise an affinity reagent and a detection modality.

A probe or an affinity reagent may comprise a functional moiety; a solubilizing moiety such as uronic acid or a phosphoryl group; a detection modality such as a fluorescent dye; a purification or affinity tag, for example biotin, an enzyme substrate, a protein agonist, or a peptide N-terminal affinity tag, such as a FLAG tag or a HIS tag; a reactive handle for chemical coupling, such as an alkyne configured for click chemistry coupling to an azide; a localization signal, such as a nuclear localization signal; a greasy group, such as a lipid or alkane; or any combination thereof. A probe may comprise a plurality of affinity reagents.

An affinity reagent or a probe may comprise an activatable functional moiety, such as a photoswitchable, photocleavable, or chemically cleavable moiety. An activatable functional moiety may include a biopolymer (e.g., a peptide or nucleic acid) or a molecule capable of adopting multiple conformations. For example, an activatable functional moiety may include a nucleic acid that changes conformations upon binding to a divalent cation, such that the probe comprises a first (e.g., an active) conformation in the presence of the divalent cation and a second (e.g., an inactive) conformation in the absence of the divalent cation. The probe may comprise a first set of analyte affinities or binding specificities in the first conformation and a second set of analyte affinities or binding specificities in the second conformation.

Affinity reagents and probes can be coupled to different detection modalities. A library of probes or affinity reagents may comprise a plurality of detection modalities that uniquely identify individual affinity reagents or probes, or that uniquely identify groups of affinity reagents or probes. For example, a library of probes may comprise nucleic acid barcodes which uniquely identify each separate type of probe within the library.

Affinity reagents and probes may be comprised of multiple distinct chemical species. For example, an affinity reagent or a probe may comprise an amino acid, a nucleotide, a biopolymer (e.g., a polysaccharide, a peptide, or a nucleic acid molecule), a small molecule, an inorganic complex, a material (e.g., a carbon nanotube), or a substrate (e.g., a nanoparticle). An affinity reagent or a probe may comprise a polymeric region, such as a biopolymer or a biopolymer-synthetic molecule conjugate (e.g., a polymer comprising alternating amino acid residue and gamma-aminobutyric acid subunits). In some cases, an affinity reagent or a probe comprises an oligonucleotide or a polynucleotide. In some cases, an affinity reagent or a probe comprises an oligopeptide or polypeptide. In some cases, an affinity reagent or a probe comprises a synthetic polymer, such as polyethylene oxide. In some cases, an affinity reagent or a probe comprises a supramolecular complex, comprised of a plurality of noncovalently or weakly covalently associated molecules, such as an antibody light chain-heavy chain conjugate. In some cases, an affinity reagent or a probe comprises a moiety that affects its physicochemical properties, such as solubility, melting temperature, or charge.

A probe may comprise an antibody. An affinity reagent may comprise or consist of an antibody. As used herein, the term antibody may refer to an immunoglobulin protein or a portion or derivative thereof, and encompasses monoclonal antibodies, multispecific antibodies, human antibodies, humanized antibodies, camelid antibodies, diabodies, chimeric antibodies, single chain Fvs (scFvs), single chain Fab fragments (scFab), nanobodies, heavy chain variable domains, single domain antibodies, Fab fragments, and portions and derivatives thereof. An antibody may comprise a complex of multiple proteins, such as a light chain and a heavy chain. A light chain-heavy chain pair may comprise a fragment antigen-binding (Fab) comprising a plurality of complementarity determining regions. In many cases, the Fab comprises binding affinity for a target molecule. An antibody may comprise a plurality of Fab regions comprising identical or distinct target affinities. For example, an antibody may comprise a dimer, tetramer, or pentamer of light chain-heavy chain pairs, each comprising a Fab. A plurality of antibody protein subunits (e.g., a light chain and a heavy chain) may be coupled by disulfide bonds. Two heavy chain constant regions may couple to form a fragment crystallizable region, which may comprise high solubility, an affinity for particular cell receptors, and multiple glycosylation sites.

An affinity reagent may comprise or consist of an antibody. An affinity reagent may comprise or consist of an antibody fragment. The antibody or antibody fragment may be humanized.

In some aspects, the affinity reagent comprises an antibody or antibody fragment comprising a single chain variable fragment (scFv), a single domain antibody (sdA), a Fab, or a Fab′. In some aspects, the antibody or antibody fragment comprises a scFv. In some aspects, the antibody or antibody fragment comprises a sdA. In some aspects, the antibody or antibody fragment comprises a Fab. In some aspects, the antibody or antibody fragment comprises a Fab′. In some aspects, the antibody or antibody fragment consists of a scFv. In some aspects, the antibody or antibody fragment consists of a sdA. In some aspects, the antibody or antibody fragment consists of a Fab. In some aspects, the antibody or antibody fragment consists of a Fab′.

In some aspects, the affinity reagent comprises an antibody or antibody fragment comprising or consisting of a Fab. In some aspects, the Fab or Fab′ comprises a Fab light chain polypeptide and a Fab heavy chain polypeptide. In some aspects, the Fab comprises a Fab light chain polypeptide. In some aspects, the Fab comprises a Fab heavy chain polypeptide. In some aspects, the Fab′ comprises a Fab light chain polypeptide. In some aspects, the Fab′ comprises a Fab heavy chain polypeptide. In some aspects, the Fab of the affinity reagent includes a light or heavy chain with a CDR that binds to a biomolecule.

In some aspects, the affinity reagent comprises an antibody or antibody fragment comprising or consisting of a sdA. In some aspects, the sdA comprises a variable domain of a heavy chain polypeptide. In some aspects, the sdA comprises a variable domain of a lambda light chain polypeptide. In some aspects, the sdA comprises a variable domain of a kappa light chain polypeptide. In some aspects, the sdA comprises a variable domain of a heavy chain polypeptide, a variable domain of a lambda light chain polypeptide, or a variable domain of a kappa light chain polypeptide. In some aspects, the sdA of the affinity reagent includes a CDR that binds to a biomolecule.

In some aspects, the affinity reagent comprises an antibody or antibody fragment comprising or consisting of a scFv. In some aspects, the scFv comprises a scFv heavy chain variable domain. In some aspects, the scFv comprises a scFv light chain variable domain. In some aspects, the scFv comprises a scFv heavy chain variable domain and a scFv light chain variable domain. In some aspects, the scFv of the affinity reagent includes a CDR that binds to a biomolecule.

In some cases, a probe may comprise a linker. The term “linker” may refer to a chemical structural unit that connects two or more distinct moieties. A linker may comprise a nucleotide, an amino acid, a nucleic acid, a peptide, a small molecule, an oligomer, a polymer, or a derivative or any combination thereof, such as a 2-methoxyethan-1-amino linker. A linker may comprise a synthetic polymer such as ethylene oxide. In some cases, a linker may be cleavable (e.g., hydrolysable sulfone linkers). A linker may have a defined chemical structure, or may have the flexibility to adopt multiple conformations. A linker may affect the physicochemical properties of an affinity reagent to which it is coupled.

In some cases, an affinity reagent or a probe comprises multiple moieties or segments with different physicochemical properties. In some cases, the affinity reagent itself binds to a target (e.g., a target protein on the surface of a particle). An affinity reagent may comprise multiple moieties that bind to a target (e.g., multiple epitopes on a single protein). In such cases, two or more of the moieties may bind the same target. An affinity reagent may comprise multiple moieties that bind different targets. An affinity reagent consistent with the present disclosure may comprise an antibody, a peptide, a nucleic acid affinity reagent, a Fab, a Fab2, an scFv, an scFab, an aptamer, a polypeptide affinity reagent scaffold, or a chemical moiety. A polypeptide affinity reagent scaffold may comprise any number of polypeptide affinity reagent scaffolds capable of binding to a target, such as an adnectin, abamer, affibody, or nanobody. In some cases, affinity reagent binding comprises non-covalent interactions. In such cases, binding affinity for a target may be driven by electrostatic forces, such as van der Waals interactions. In some cases, affinity reagent binding comprises covalent bond formation between the affinity reagent and a target.

In some cases, an affinity reagent comprises a linear arrangement of chemical species. An affinity reagent may comprise a heteropolymer comprised of different molecular units. For example, an affinity reagent could have the chemical formula X₁—Y₁—X₂—X₁—N—X₃—Y₂—L—A, where X₁-X₃ denote oligopeptides, Y₁ and Y₂ are phospholipids, N is a polynucleotide, L is a branched polyethylene glycol, A is the inorganic complex ferrocenium, and each ‘-’ is either a bond or a chemical linker. The partial or complete identity of an affinity reagent can sometimes be determined from the sequence of one or more polymeric subunits. For example, an affinity reagent may comprise or consist of an aptamer which may be sequenced for identification. As used herein, an aptamer may be a nucleic acid molecule (e.g., a deoxyribonucleic acid (DNA) or ribonucleic acid (RNA)) which comprises a binding affinity for a target molecule.

A probe may also have segments or moieties that do not bind targets. In some cases, a segment of a probe may serve as a detection modality. In some cases, a detection modality comprises a chemically recognizable marker. For example, a detection modality may comprise a detectable label such as an optically detectable dye or a reducible and electrochemically detectable marker. A detection modality may comprise a polymeric segment with a recognizable sequence. For example, a detection modality may comprise an oligonucleotide, a polynucleotide, an oligopeptide, or a polypeptide with an identifiable sequence. A detection modality may comprise a nucleotide, which may contain coding and noncoding regions. The nucleotide may also contain a barcoding sequence, which may be used to identify partial or complete chemical and structural characteristics of the probe. A detection modality may also comprise a moiety that facilitates its collection or isolation. For example, a detection modality may comprise a biotin moiety that can be captured by streptavidin, a charged moiety for electrophoretic separation, a magnetic moiety that allows for magnetic capture, or a reactive moiety, such as a maleimide, that allows the affinity reagent to couple to a capture species.

Affinity reagent binding specificity for a particular target may be sensitive to the structural state of the target (e.g., a protein present in the corona of a particle as disclosed herein). In some cases, an affinity reagent will have binding affinity for a particular conformation of a target. Accordingly, a method may utilize affinity reagent or probe binding to detect a conformation of a biomolecule. For example, a probe-binding assay may identify a ratio of activated and deactivated rhodopsin in a sample.

An affinity reagent may be sensitive to chemical modifications of a target. For example, an affinity reagent’s binding specificity for a protein may be affected by post-translational modification of the protein. Some non-limiting examples of post-translational modifications include glycosylation, acetylation, alkylation, biotinylation, glutamylation, glycylation, isoprenylation, phosphorylation, lipolation, phosphopantetheinylation, sulfation, selenation, amidation, ubiquitination, hydroxylation, nitrosylation, or SUMOylation. An affinity reagent may be sensitive to the protonation state of a target. For example, an affinity reagent may have a high binding affinity for a target below pH 5.0 and a low binding affinity for the same target above pH 5.5. An affinity reagent may be sensitive to a conformation of a target species. An affinity reagent may comprise at least 1-, at least 2-, at least 3-, at least 4-, or at least 5-orders of magnitude higher binding affinity for a target species when the target species is in a first conformation rather than a second conformation.

Affinity reagents may be sensitive to sequence variations in target proteins or nucleic acids. An affinity reagent may comprise a binding affinity for a protein mutant. An affinity reagent may comprise a binding specificity for a single splicing variant of a protein. An affinity reagent may comprise binding affinities for multiple splicing variants of a protein. A plurality of affinity reagents may each separately bind to different splicing variants of a protein. Similarly, an affinity reagent may have different binding affinities for different protein isoforms.

Probes consistent with the compositions and methods disclosed herein may comprise a range of sizes. A probe may have a mass of less than 1 kilodalton (kDa). A probe may have a mass of at least 2 kDa. A probe may have a mass of at least 3 kDa. A probe may have a mass of at least 4 kDa. A probe may have a mass of at least 5 kDa. A probe may have a mass of at least 10 kDa. A probe may have a mass of at least 20 kDa. A probe may have a mass of at least 30 kDa. A probe may have a mass of at least 40 kDa. A probe may have a mass of at least 50 kDa. A probe may have a mass of at least 60 kDa. A probe may have a mass of at least 80 kDa. A probe may have a mass of at least 100 kDa. A probe may have a mass of at least 150 kDa. A probe may have a mass of at least 200 kDa. A probe reagent may have a mass of at least 250 kDa. A probe reagent may have a mass of at least 500 kDa. A probe may have a mass of at most 500 kDa. A probe may have a mass of at most 250 kDa. A probe may have a mass of at most 200 kDa. A probe may have a mass of at most 150 kDa. A probe may have a mass of at most 100 kDa. A probe may have a mass of at most 80 kDa. A probe may have a mass of at most 60 kDa. A probe may have a mass of at most 50 kDa. A probe may have a mass of at most 40 kDa. A probe may have a mass of at most 30 kDa. A probe may have a mass of at most 20 kDa. A probe may have a mass of at most 10 kDa. A probe may have a mass of at most 5 kDa. A probe may have a mass of at most 4 kDa. A probe may have a mass of at most 3 kDa. A probe may have a mass of at most 2 kDa. A probe may have a mass of at most 1 kDa.

Hydrodynamic radius, which is herein defined as the radius of a hard sphere that would diffuse at the same rate as a molecule under observation, can be a useful measure of a molecule’s physical size. The present disclosure provides probes spanning a wide range of dimensions. A probe may be comparable in size or larger than a typical antibody. A probe reagent may have a hydrodynamic radius of at least 1 nm. A probe may have a hydrodynamic radius of at least 2 nm. A probe may have a hydrodynamic radius of at least 3 nm. A probe may have a hydrodynamic radius of at least 4 nm. A probe may have a hydrodynamic radius of at least 5 nm. A probe may have a hydrodynamic radius of at least 6 nm. A probe may have a hydrodynamic radius of at least 7 nm. A probe may have a hydrodynamic radius of at least 8 nm. A probe may have a hydrodynamic radius of at least 9 nm. A probe may have a hydrodynamic radius of at least 10 nm. A probe may have a hydrodynamic radius of at least 11 nm. A probe may have a hydrodynamic radius of at least 12 nm. A probe may have a hydrodynamic radius of at least 15 nm. A probe may have a hydrodynamic radius of at least 20 nm. A probe may have a hydrodynamic radius of at least 25 nm. A probe may have a hydrodynamic radius of at least 20 nm. A probe may have a hydrodynamic radius of at most 25 nm. A probe may have a hydrodynamic radius of at least 20 nm. A probe may have a hydrodynamic radius of at most 20 nm. A probe may have a hydrodynamic radius of at least 20 nm. A probe may have a hydrodynamic radius of at most 15 nm. A probe may have a hydrodynamic radius of at least 20 nm. A probe may have a hydrodynamic radius of at most 10 nm. A probe may have a hydrodynamic radius of at least 20 nm. A probe may have a hydrodynamic radius of at most 8 nm. A probe may have a hydrodynamic radius of at least 20 nm. A probe may have a hydrodynamic radius of at most 6 nm. A probe may have a hydrodynamic radius of at least 20 nm. A probe may have a hydrodynamic radius of at most 5 nm. A probe may have a hydrodynamic radius of at least 20 nm. A probe may have a hydrodynamic radius of at most 4 nm. A probe may have a hydrodynamic radius of at least 20 nm. A probe may have a hydrodynamic radius of at most 3 nm. A probe may have a hydrodynamic radius of at least 20 nm. A probe may have a hydrodynamic radius of at most 2 nm. A probe may have a hydrodynamic radius of at least 20 nm. A probe may have a hydrodynamic radius of at most 1 nm.

A probe may be smaller than a typical antibody. A probe may have a hydrodynamic radius of around 5 nm. A probe may have a hydrodynamic radius of around 4 nm. A probe may have a hydrodynamic radius of around 3 nm. A probe may have a hydrodynamic radius of around 2 nm. A probe may have a hydrodynamic radius of around 1 nm. A probe may have a hydrodynamic radius of around 0.5 nm. A probe may have a hydrodynamic radius of around 0.25 nm. A probe may have a hydrodynamic radius of between 1 and 5 nm. A probe may have a hydrodynamic radius of between 1 and 3 nm. A probe may have a hydrodynamic radius of between 3 and 5 nm.

Small probe sizes offer a number of potential advantages for assaying biomolecules. Binding assays are sometimes limited by steric constraints, which can prevent multiple probes from binding to closely spaced targets. This problem can be especially pronounced in assays that utilize antibodies, which have fairly large hydrodynamic radii. The use of probes with diminutive sizes can allow more probes to bind targets within a spatially limited area. In some cases, this allows more probes to bind to a particular biomolecule or supramolecular complex. For example, a greater number of probes from the present disclosure may be able to bind to a biomolecule corona surrounding a particle than could be accomplished with antibodies. A plurality of probes of the present disclosure may be able to have 1.5 times, 2 times, 3 times, 4 times, 5 times, 10 times, 20 times, 30 times, 40 times, 50 times, or 100 or more times as many probes over a defined area than could be accomplished with large probes, such as antibodies. For example, a 100 nm diameter particle comprising a 3×10⁴ nm² surface area may be able to accommodate at most 500 antibodies on its surface (or disposed on the surface of a biomolecule corona bound to its surface), but over 5000 probes with radii of about 1 nm. Accordingly, a small probe may generate a greater degree of profiling depth than a large probe.

A probe may comprise a broad or narrow range of specificities for biomolecules from a sample (e.g., a human plasma sample). A probe may comprise an affinity for a single species (e.g., a biomolecule) or family of species (e.g., cadherin family proteins) from a sample. In such cases, the probe may comprise a binding affinity (e.g., a dissociation constant, K_{D}) of at least 1.5, at least 2, at least 2.5, at least 3, at least 3.5, at least 4, at least 4.5, or at least 5 orders of magnitude greater for its target than for other species in the sample. The probe may comprise a binding affinity of at most 5, at most 4.5, at most 4, at most 3.5, at most 3, at most 2.5, or at most 2 orders of magnitude greater for its target than for other species from the sample. When contacted to the sample at least 30%, at least 35%, at least 40%, at least 50%, at least 60%, at least 70%, at least 80%, at least 90%, at least 95%, at least 98%, at least 99%, at most 99%, at most 98%, at most 95%, at most 90%, at most 80%, at most 70%, at most 60%, at most 50%, or at most 40% of the binding by the affinity of the reagent may be to the target species. The probe may comprise a binding affinity (e.g., measured as a K_{D} for its target) of at most 1 µM, at most 100 nM, at most 10 nM, at most 1 nM, at most 100 pM, at most 10 pM, or at most 1 pM for its target. The probe may comprise a binding affinity of at least 100 nM, at least 10 nM, at least 1 nM, at least 100 pM, at least 10 pM, or at least 1 pM for its target.

A probe may comprise specificities for a plurality of species (e.g., unique biomolecules or classes of biomolecules, such as protein families) in a sample. The probe may comprise at least 2 target species, at least 3 target species, at least 4 target species, at least 5 target species, at least 6 target species, at least 8 target species, at least 10 target species, at least 12 target species, at least 15 target species, at least 20 target species, at least 25 target species, at least 30 target species, at least 40 target species, at least 50 target species, at least 80 target species, at least 100 target species, at least 150 target species, at least 200 target species, at least 250 target species, at least 300 target species, at least 400 target species, at least 500 target species, at least 600 target species, at least 800 target species, or at least 1000 target species. The probe may comprise at most 3 target species, at most 4 target species, at most 5 target species, at most 6 target species, at most 8 target species, at most 10 target species, at most 12 target species, at most 15 target species, at most 20 target species, at most 25 target species, at most 30 target species, at most 40 target species, at most 50 target species, at most 80 target species, at most 100 target species, at most 150 target species, at most 200 target species, at most 250 target species, at most 300 target species, at most 400 target species, at most 500 target species, at most 600 target species, at most 800 target species, or at most 1000 target species. The probe may comprise specificities for a group or class of species from the sample. For example, the probe may comprise specificities for immunoglobulin domains, and thereby appreciably bind to a range of antibody, interleukin receptor, and signaling (e.g., lectin) proteins. The probe may comprise binding affinities of at least 1, at least 1.5, at least 2, at least 2.5, at least 3, at least 3.5, at least 4, at least 4.5, or at least 5 orders of magnitude greater for its targets than for other species in the sample. The probe may comprise binding affinities of at most 1, at most 1.5, at most 2, at most 2.5, at most 3, at most 3.5, at most 4, at most 4.5, or at most 5 orders of magnitude greater for its targets than for other species in the sample. Probe specificity may be defined by a benchmark binding affinity strength. The probe may comprise binding affinities of at most 1 mM, at most 100 µM, at most 10 µM, at most 1 µM, at most 100 nM, at most 10 nM, at most 1 nM, at most 100 pM, at most 10 pM, or at most 1 pM for its targets. The probe may comprise binding affinities of at least 1 mM, at least 100 µM, at least 10 µM, at least 1 µM, at least 100 nM, at least 10 nM, at least 1 nM, at least 100 pM, at least 10 pM, or at least 1 pM for its targets. When contacted to the sample, at least 5%, at least 10%, at least 15%, at least 20%, at least 25%, at least 30%, at least 35%, at least 40%, at least 50%, at least 60%, at least 70%, at least 80%, at least 90%, at least 95%, at least 98%, at least 99%, at most 99%, at most 98%, at most 95%, at most 90%, at most 80%, at most 70%, at most 60%, at most 50%, at most 40%, at most 35%, at most 30%, at most 25%, at most 20%, at most 15%, at most 10%, or at most 5% of the binding by the affinity of the reagent may be to its target species. A probe may comprise relatively low binding affinities for species in a sample. For example, probe may comprise a binding affinity of at least 100 µM, at least 200 µM, at least 300 µM, at least 400 µM, at least 500 µM, at least 600 µM, at least 800 µM, or at least 1 mM for the species present in a sample, and thereby non-specifically and in some cases transiently bind to a wide range of species from the sample.

Any of the probes described above can be coupled to a detection modality disclosed herein and combined with the methods of assaying for a protein in the corona of a particle disclosed herein. The presence or abundance (e.g., concentration) of the probe may be determined by the presence or intensity of a signal from the detection modality. For example, a particle disclosed herein may be incubated in a sample allowing for biomolecules (e.g., proteins) in the sample to adsorb to the surface of the particle, thereby forming a biomolecule corona. The particle having a corona of proteins is then incubated with one or more of the probes disclosed herein. The probe is coupled to a detection modality (e.g., a substrate for an enzyme for a colorimetric readout, a fluorophore for a fluorescent readout, or a nucleic acid sequence that can be optionally amplified and sequenced by next generation sequencing). If the probe’s target is present in the corona of the particle, the probe binds the target and thereby the particle. The probe may optionally be decoupled from the detection modality. For example, a nucleic acid barcode may be cleaved from a probe collected on a biomolecule corona. For many of the methods herein that include use of a probe comprising a barcode, the method may alternatively include use of a probe comprising a detection modality other than a barcode. A signal from the detection moiety is assayed for, thereby assaying for the presence or absence of the protein. Advantageously, this method allows for proteomic analysis without the need for mass spectrometry.

### Probe and Affinity Reagent Libraries

Various aspects of the present disclosure provide libraries (e.g., a DNA encoded library) comprising a plurality of probes. The plurality of probes may vary in their structural, chemical, and physical properties, such as target binding affinity. Depending on the application required, a library of probes can comprise fewer than 10 probes or greater than 10⁹ probes. In some cases, a library of probes comprises about 10 probes. In some cases, a library of probes comprises about 10² probes. In some cases, a library of probes comprises about 10³ probes. In some cases, a library of probes comprises about 10⁴ probes. In some cases, a library of probes comprises about 10⁵ probes. In some cases, a library of probes comprises about 10⁶ probes. In some cases, a library of probes comprises about 10⁷ probes. In some cases, a library of probes comprises about 10⁸ probes. In some cases, a library of probes comprises about 10⁹ probes. In some cases, a library of probes comprises about 10¹⁰ probes. A library of probes may comprise multiple identical probes. Each member of a library of probes may be a unique type of probe.

A library of probes may comprise probes with common structural motifs. For example, all members of a library of probes may have the structure B-B-B-N^{a}-B-B-B-C, where each instance of B can be selected from a wide range of chemical species, N^{a} is a 100 nucleotide length nucleic acid whose sequence varies among members of the library, and C is biotin.

In some cases, a plurality of different types of probes from among a plurality of probes will each have a unique label. For example, a library of probes comprising 10⁸ types of probes may comprise 10⁸ different labels, each uniquely associated with a particular type of probe. In some cases, the labels comprise optically detectable (e.g., fluorescent or luminescent) moieties.

In some cases, an probe library can comprise a DNA encoded library. As used herein, a DNA encoded library may refer to a library of molecules that comprise identifying nucleic acid sequences. An example of a DNA encoded library is a library of mRNA display translation products, which comprises nucleotides coupled to the peptides which they encode. A DNA encoded library may comprise synthetic constructs, such as nucleic acid-small molecule or nucleic acid-lipid conjugates. In such cases, the nucleic acids may comprise sequences which identify the molecules bound to the nucleic acids. A DNA encoded library may include cyclic constructs. A DNA encoded library may include a library of nucleic acids (single stranded or double stranded) coupled to one or more small molecules. A DNA encoded library may comprise probes which comprise an identifiable nucleic acid sequence (e.g., nucleic acid barcodes) and a nucleic acid moiety that imparts activity (e.g., a deoxyribozyme unit) or binding affinity (e.g., an aptamer). A DNA encoded library may comprise a molecule that consist entirely of nucleic acids. A DNA encoded library may be a combinatorial self-assembling library, such as an encoded self-assembling chemical library (ESAC library). Alternatively, a DNA encoded library may comprise nucleic acids that have been conjugated to small molecules or other organic molecules.

DNA encoded libraries may offer the ability to determine the full structure of a library member. A member of a DNA encoded library may contain a nucleic acid sequence that provides information on its composition and structure. For example, a DNA encoded library may comprise molecules of the general structure M₁—M₂—M₃—M₄—N, wherein M₁-M₄ are each independently selected from a set of small molecules and N is a nucleic acid with a first sequence that identifies M₁, a second sequence that identifies M₂, a third sequence that identifies M₃, and a fourth sequence that identifies M₄.

In some cases, DNA encoded library synthesis is mediated by the nucleic acid sequences of the member species. For example, an probe library may comprise a DNA encoded library which comprises nucleic acids coupled to the polypeptides for which they encode. DNA encoded libraries may also be constructed with sequence specific affinity capture methods, utilizing sequential coupling steps which comprise collecting all members of a library containing a particular nucleic acid sequence, reacting or modifying each of the collected library members (e.g., appending a particular moiety at the 5′ end of each collected member), and repeating. DNA encoded libraries may be constructed with nucleic acid templated synthesis methods, wherein hybridization events between template nucleic acids and reagent loaded nucleic acids result in terminal modifications or appendages on the template strand.

In some cases, each type of probe may have a unique nucleic acid barcode. For such a library, a plurality of probes can quickly be identified by sequencing the nucleic acid barcode for each probe present (e.g., with any number of next-generation sequencing (NGS) methods). In some cases, nucleic acid barcode sequencing can determine the types of probes present and the relative amounts of each type of probe present.

### Probe and Affinity Reagent Library Generation

Aspects of the present disclosure provide probe libraries and methods for generating probe libraries comprising pluralities of types of probes with unique structural and chemical properties. In some cases, each type of probe of a plurality of probes comprises a unique identifiable label. In some cases, each probe comprises a unique type of identifiable label. In some cases, the unique identifiable label is a nucleic acid sequence. In some cases, the unique identifiable label provides information as to the probe composition or structure.

Large probe libraries with unique nucleic acid barcode sequences may be combinatorially generated from small libraries of nucleic acids. A method for generating a library of unique nucleic acid barcodes can comprise the sequential ligation of a small number of nucleic acid sequences. For example, a nucleic acid barcode library with more than 5×10¹⁰ unique members can be generated by constructing 18-mer nucleic acids from a pool of 6-mer nucleic acids with random sequences. Similarly, large nucleic acid libraries can be prepared through iterative recombination of a smaller input library.

Probe libraries may be used in directed evolution processes to generate probe and subsequent probe libraries with tailored properties, such as binding specificities and affinities, reactivities, melting temperatures, solubilities, stabilities, sizes, catalytic activity, agonist activity, antagonist activity, and solvent and pH tolerances. Directed evolution schemes may utilize unique barcoding for each type of probe in a library of probe. In each step, probes with desired affinities, activities, or properties can be collected and identified based on their labels (e.g., nucleic acid barcodes), and then used to generate a new library of probes. A directed evolution process may comprise multiple iterations of such a selection process. Examples of directed probe library generation techniques consistent with the present disclosure include mRNA display, DNA templated synthesis, DNA routing, error-prone PCR, split-and-pool synthesis, DNA-walker based synthesis, hybridization chain reactions, and encoded self-assembled library synthesis.

A probe or affinity reagent may be generated through positive selection, negative selection, or a combination thereof. Positive selection may comprise contacting a probe or affinity reagent to a molecule or sample in which it is intended to bind. For example, a positive selection round for a probe for detecting Alzheimer’s disease detection may comprise contacting the probe to a biomolecule corona generated from the plasma of an Alzheimer’s patient. A negative selection round may comprise contacting a probe or affinity reagent to a molecule or sample which it is intended not to bind. For example, a negative selection round for a probe for detecting non-small cell lung cancer (NSCLC) may comprise contacting the probe to a biomolecule corona generated from the plasma of a cancer-free patient. Iterative rounds of positive selection, negative selection, or a combination thereof may be used to generate a probe which not only binds to an intended target, but also which does not bind to non-target samples or species.

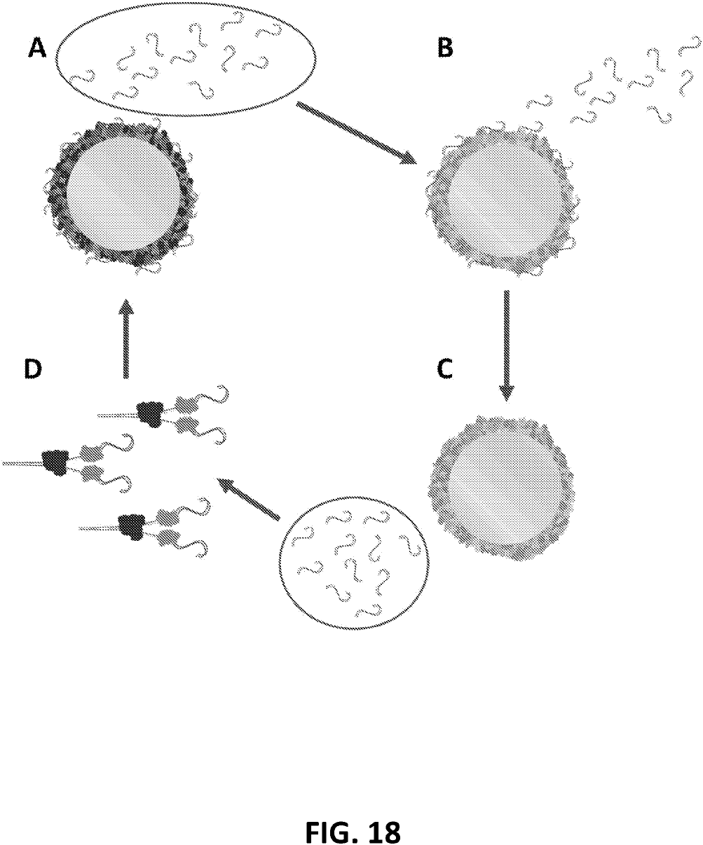

FIG. 18 provides an example of a probe library directed evolution method in which a library of aptamer probes comprising nucleic acid molecules are subjected to rounds of positive and negative selection. FIG. 18 Panel A illustrates selection of a subset of probes from the aptamer probe library which do not bind to a first biomolecule corona, thereby selecting probes comprising weak affinity for a particular sample type or biological state. FIG. 18 Panel B illustrates binding of the subset of probes to a second biomolecule corona. The probes which do not bind to the second biomolecule corona are discarded, while the probes which bind the second biomolecule corona are collected (as shown in FIG. 18 Panel C), thereby selecting probes comprising an affinity for the second biomolecule corona and lacking an affinity for the first biomolecule corona. The selected probes are amplified through error prone PCR (as shown in FIG. 18 Panel D), thereby generating a new library of probes comprising mutations relative to the subset of probes selected through the biomolecule corona binding assay to extend the aptamer sequence space queried by the library evolution method.

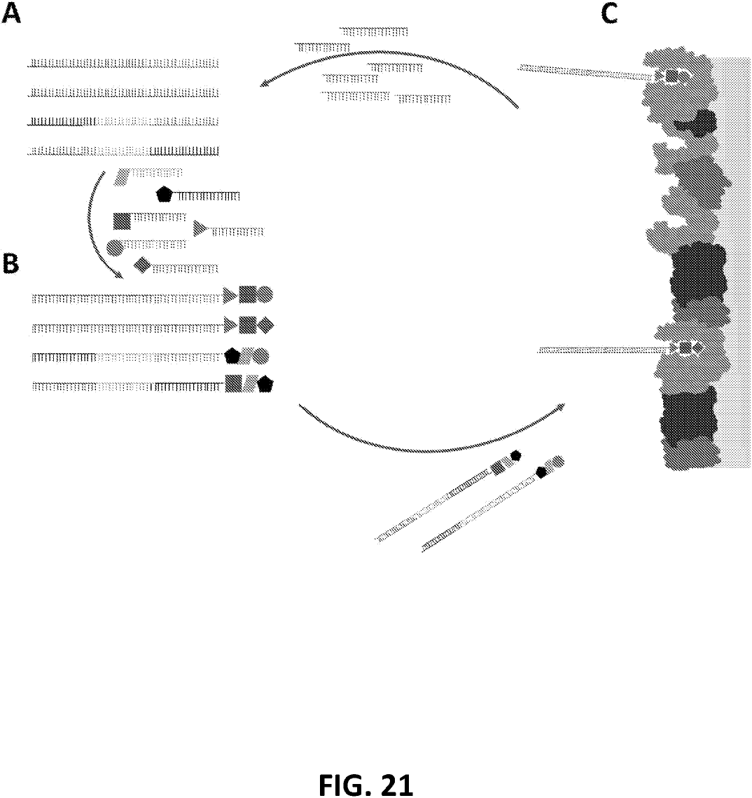

A further example of a probe evolution is provided in FIG. 21, which outlines a method utilizing biomolecule corona-affinity selection. In this example, a combinatorial library of nucleic acid barcodes is randomly assembled from small nucleic acid library comprising a number of short nucleic acid sequences (FIG. 21 Panel A). The resulting barcodes are then utilized for nucleic acid templated synthesis, in which reactive groups are transferred from a set of oligonucleotides complementary to portions of the barcodes (FIG. 21 Panel B). Multiple oligonucleotide contacting rounds may be performed to generate complex reactive group sequences appended to each barcode. As is shown in FIG. 21 Panel C, the library of reactive group-bearing barcodes may then be contacted to a biomolecule corona of a particle. A subset of barcodes may comprise reactive group combinations with affinities for a corona-bound biomolecule (e.g., affinity for an enzyme active site), and thus may adsorb to the biomolecule corona. Biomolecule corona bound barcodes can be collected, digested, amplified, reassembled to form a new barcode library. This library evolution scheme can be used to generate probes specific for a particular biomolecule (e.g., ceruloplasmin) or disease state (e.g., Wilson’s disease).

### Probe and Affinity Reagent Assays and Detection

Aspects of the present disclosure provide methods for identifying analytes (e.g., proteins within a biomolecule corona) with probes. In some cases, an analyte may be identified by binding a probe to the analyte and identifying the probe or a detection modality coupled thereto. In some cases, an analyte may be identified with a physical characterization technique, such as mass spectrometry, optical detection, or electrochemical analysis. In some cases, an analyte is analyzed with a probe and a physical characterization technique. For example, a method may comprise detecting antibody binding to a protein, fragmenting the protein, and analyzing the resulting fragments with mass spectrometry.

Any of the probes or libraries of probes described herein can be coupled to a detection modality and utilized for a biomolecule corona assay. A particle disclosed herein may be incubated in a sample allowing for biomolecules (e.g., proteins) in the sample to adsorb to the surface of the particle, thereby forming a biomolecule corona. The particle having a corona of proteins may then be incubated with one or more of the probes disclosed herein. If the probe is coupled to a detection modality, a signal from the detection moiety may assayed for to assay for the presence or absence of the probe or for one of its targets. Advantageously, this method allows for proteomic analysis without the need for mass spectrometry.

In some cases, a plurality of particle-types are contacted to a sample prior to probe analysis. As demonstrated in FIG. 5, such multiplexing may increase the number of biomolecules enriched from the sample. In some cases, a plurality of assays are performed in parallel with different particle types or ligand libraries. For example, parallel probe binding assays may be performed on a multi-well plate in which separate sample volumes are contacted to separate particle types or sample conditions (e.g., viscosity or pH), thereby producing separate probe binding patterns. In some cases, probe analysis is performed on a plurality of particles. For example, a single sample volume comprising biomolecule coronas of at least two distinct particle types may be contacted with a probe library for binding analysis.

In some cases, a method for analyzing a sample comprises contacting a biomolecule corona with a plurality of probes (e.g., a DNA encoded library), and identifying the probes that bind to the biomolecule corona. Such a method may comprise removing probes that do not bind to the biomolecule corona, for example by magnetic particle immobilized followed by wash steps, filter, or fractionation steps (e.g., chromatographically or through phase separation) to remove probes not bound to a biomolecule. The method may comprise detecting probes which bind or probes which do not bind (e.g., are collected in a wash step) to the biomolecule corona. Detection modalities coupled to the probes may be detected, sequenced, or analyzed to identify the probes which bind to the biomolecule corona. Probes bound to the biomolecule corona may be collected (e.g., eluted from the biomolecule corona) and subjected to analysis. Alternatively or additionally, the probes which do not bind to a sample may optionally be collected and may be analyzed. Detection modalities (e.g., nucleic acid barcodes or optically detectable dyes) may be cleaved from the biomolecule corona bound probes, may optionally be collected, and may be analyzed (e.g., flowed through a fluorimeter for detection). Detection may comprise detection of detection modalities of probes bound to the biomolecule corona.

A combined probe and particle assay may comprise direct biomolecule corona analysis. A method may comprise mass spectrometric analysis of a biomolecule corona subsequent to probe analysis. A method may also comprise mass spectrometric analysis of a first portion of a biomolecule corona prior to probe analysis on a second portion of the biomolecule corona. For example, subsequent to biomolecule corona formation, a ‘soft’ (e.g., weakly bound) portion of the biomolecule corona may be eluted and subjected to mass spectrometric analysis, while a remaining portion (e.g., a ‘hard’ tightly bound portion of the biomolecule corona) may be interrogated with a probe library. A method may also comprise parallel mass spectrometric and probe-based analysis. Such a method may comprise generating a first biomolecule corona for probe-based analysis, and in parallel generating a second biomolecule corona for mass spectrometric interrogation.

A combined particle and probe assay may comprise contacting a sample with a particle under conditions sufficient for biomolecule corona formation. The particle may be magnetically immobilized within the sample volume, and non-particle-bound species from the sample may be removed in a plurality of wash steps. While still immobilized, the particle may be contacted with a fluid flow comprising a plurality of probes coupled to electrochemically distinguishable detection modalities. The probes may move through the sample at a rate dependent upon biomolecule corona binding, such that probes which do not comprise binding specificities for biomolecule corona species may move through the sample faster tha probes comprising moderate or high binding affinities for biomolecule corona species. A faradaic detector may generate electrochemical signals from the detection modalities of probes leaving the sample volume. In some cases, the relative rates of probe transit through the sample may be used to determine aspects of the biomolecule corona composition, which may further be used to identify a biological state of the sample. In other cases, the probe rates may be used to identify a biological state of the sample without identification or correlation to biomolecule corona composition. In some cases, the identifying comprises sequencing a barcode (e.g., a nucleic acid barcode) coupled to a probe. Such a method may comprise cleaving the barcode from the probe prior to barcode analysis (e.g., barcode sequencing).

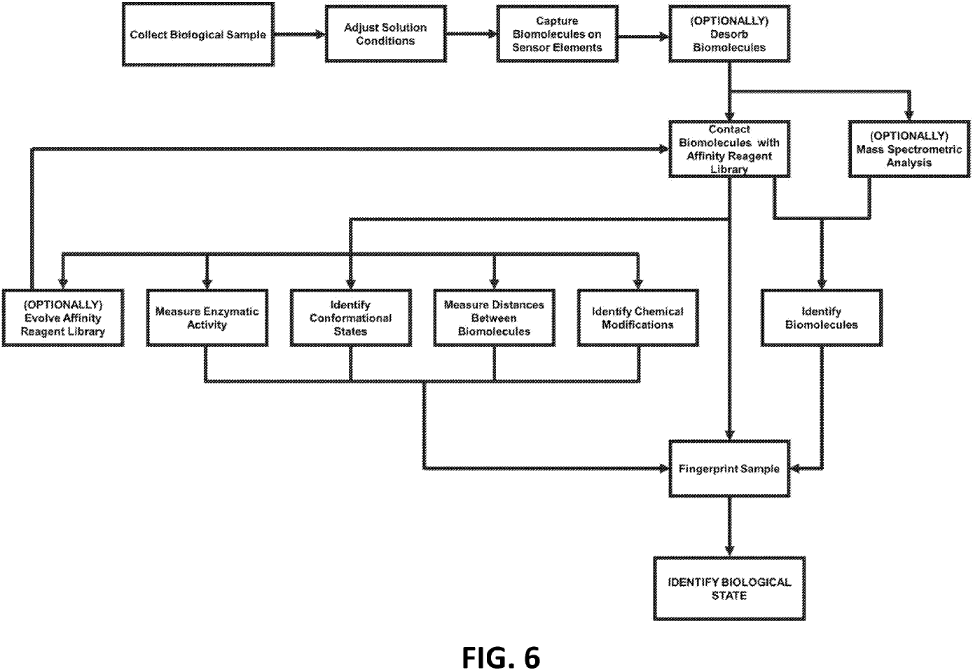

FIG. 6 provides a workflow for a proteomic analysis method consistent with the present disclosure. Once a biological sample has been collected, the solution conditions (e.g., pH, ionic strength, dielectric constant, surface tension, etc.), are adjusted to optimize biomolecule-biomolecule and biomolecule-sensor element interactions for the particular assay. The sample is then contacted to a sensor element (e.g., a polymer matrix) or an array of sensor elements (e.g., a particle array), resulting in biomolecule capture on the sensor elements (e.g., biomolecule corona formation on a particle). All or a portion of the captured biomolecules may then optionally be desorbed from the sensor element(s). For example, the soft corona portion of a biomolecule corona may be desorbed and collected for analysis. This assay utilizes probe binding analysis and optionally mass spectrometric analysis to obtain information from a sample. Either method may be used to determine the identity of biomolecules that bound to a particular sensor element.

Probe binding may also be used to obtain chemical and physical information regarding a sample. A probe library may be used to determine chemical modifications on species within a sample. This can be performed in a target-blind manner (e.g., determining whether the sample contains a phosphotyrosine), or in a target-specific manner (e.g., quantifying the ratio of inactive to GTP-activated KRAS in a sample). Probe binding may be used to measure the distances between two molecular species. A library of probes may contain an array of probes with different distance requirements for proximity extension or proximity ligation, thus allowing the probe pool to act as a molecular ruler. Intermolecular distance measurements may be used to identify an array of sample characteristics, including protein-protein interactions, protein-small molecule interactions, and protein conformation. Protein conformation may also be measured by conformation-specific probes (e.g., an antibody with a paratope for a protein surface that is only accessible when the protein is in a particular conformational state). Probes may also be used to measure enzymatic activity. For example, a probe may contain an enzyme-substrate that converts to a target-binding moiety in the presence of a particular activated enzyme.

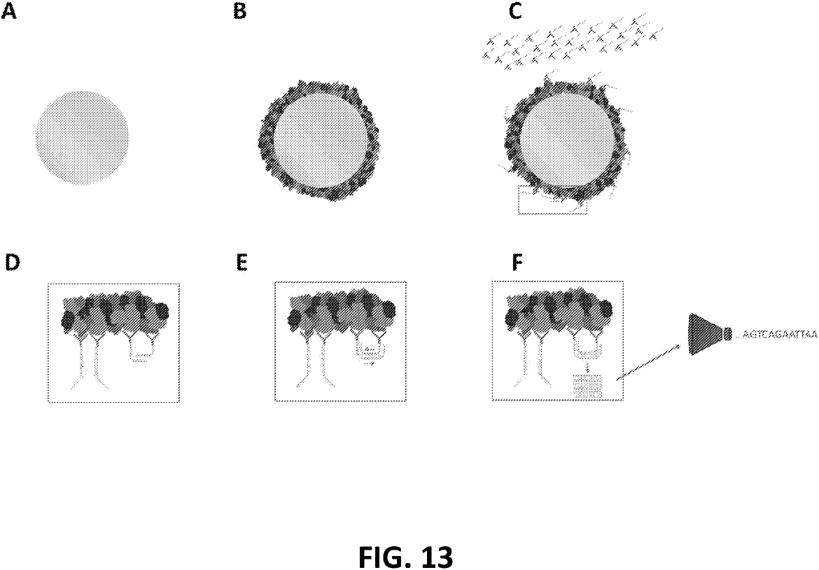

A probe or plurality of probes may be used to measure distances between biomolecules. FIG. 13 provides an example of a proximity extension assay on a biomolecule corona. FIG. 13 Panel A shows a bare particle prior to the particle contacting a sample. FIG. 13 Panel B shows the particle following biomolecule corona formation after the particle has been contacted with a sample. FIG. 13 Panel C shows the particle being contacted by a library of nucleic acid barcoded antibodies, wherein a subset of the nucleic acid barcoded antibodies bind to biomolecules on the surface of the biomolecule corona, and the remainder are washed away. FIG. 13 Panels D-F provide a closeup view of the surface of the biomolecule corona. FIG. 13 Panel D shows a pair of closely spaced antibodies with mismatching nucleic acid barcodes (left) and a pair of closely spaced antibodies with partially matching nucleic acid barcodes which have hybridized (right). FIG. 13 Panel E shows the hybridized nucleic acid barcodes undergoing extension. FIG. 13 Panel F shows the extension product from Panel E undergoing amplification and sequencing.

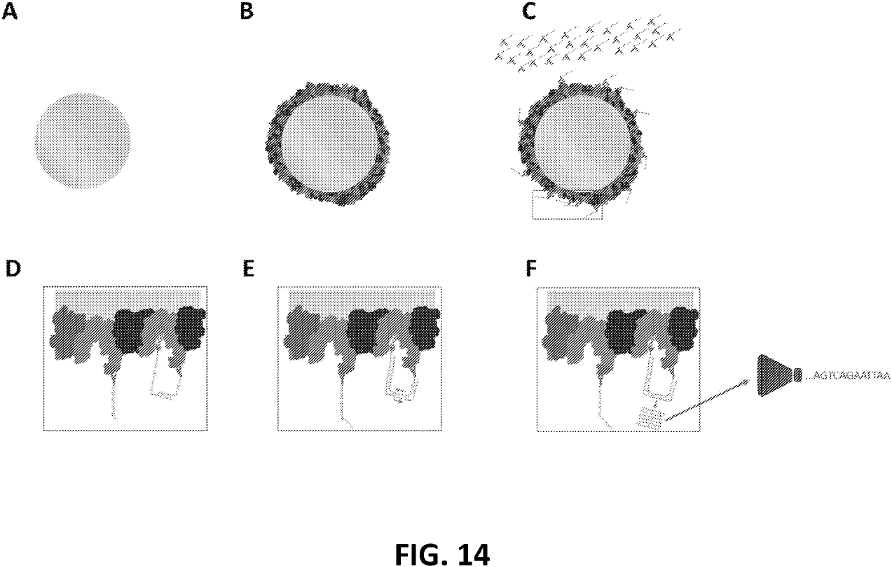

FIG. 14 provides a further example of a biomolecule corona-based proximity extension assay. In this illustration, probes comprising nucleic acid barcodes are coupled to a protein and a substrate bound to a protein active site. FIG. 14 Panel A shows a bare particle prior to contacting a sample. FIG. 14 Panel B shows the particle after it has contacted the sample and a biomolecule corona has formed on its surface. The particle is then contacted with a library of probes (e.g., a DEL or antibody library), as shown in FIG. 14 Panel C. FIG. 14 Panel D provides a closeup view where three probes are bound to the biomolecule corona. Each probe contains a target binding moiety and a single stranded nucleic acid barcode. The library of probes used in this assay contains probes that bind small molecule targets and probes that bind peptide epitopes. When two probes with complementary nucleic acid barcodes bind within sufficient proximity (e.g., when a small molecule that is the target of a first probe is bound to a protein that is the target of a second probe), the barcodes can hybridize. As is shown in FIG. 14 Panel E, this enables extension of the nucleic acid barcodes. In a subsequent amplification step shown in FIG. 14 Panel F, only nucleic acid barcodes that underwent extension produce amplicons. The amplicons may be detected by NGS, indicating which pairs of probes bound to biomolecules that were within close proximity within the sample

A method may include probe library evolution. Probes that bind to the sample may be collected, analyzed, and modified through an evolutionary step. The result of such a process can yield an affinity library with improved sensitivity for a particular target or biological state. Library evolution may also refine a library’s ability to distinguish similar biological states (e.g., stage 1 vs stage 2 cancer). Once a library has been sufficiently evolved, it may be used in a range of assays.

The combination of probe binding data (and optionally, mass spectrometric data) may be combined to fingerprint a biological sample, and the fingerprint may be used to identify the biological state(s) of the sample. An advantage of the present assay is that the high dimensionality of data obtained from the assay allows a wide range of disparate variables to be correlated. The fingerprint includes not only the raw data from the assay, but correlations between the individual data. For example, low concentrations of prealbumin, prothrombin, or β₂-glycoprotein I may not be meaningful individually. However, simultaneously low concentrations of all three proteins may be correlated with cirrhosis of the liver.

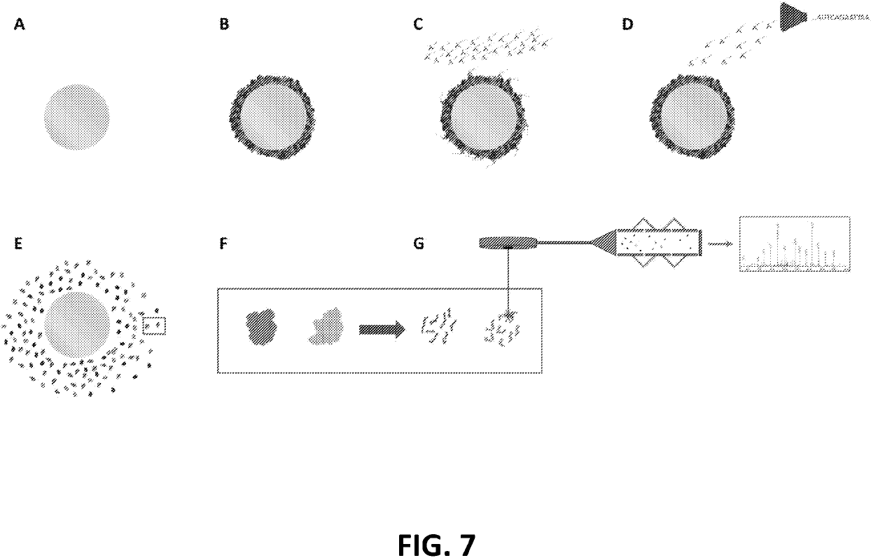

FIG. 7 illustrates a proteome analysis method that combines biomolecule corona analysis with a probe (e.g., a DNA encoded library (DEL)) binding assay. FIG. 7 Panel A shows a bare particle prior to contacting a sample. FIG. 7 Panel B shows the particle following contact with a sample and formation of a biomolecule corona. FIG. 7 Panel C shows the particle subsequently being contacted by a library of probes comprising a library of probes and nucleic acid barcodes, wherein a subset of members of the probes bind to biomolecules on the surface of the biomolecule corona, and the remainder are washed away. FIG. 7 Panel D shows the biomolecule corona-bound probes being desorbed from the corona and identified by next generation sequencing (NGS). The probes may be desorbed from the biomolecule corona, or may be desorbed from the particle along with the biomolecule corona. The NGS can determine the identities and absolute quantities of each ligand present.

FIG. 7 Panels E-G show optional steps involving mass spectrometric analysis of the biomolecule corona. FIG. 7 Panel E shows the biomolecules being desorbed from the particle. Panel F shows desorbed proteins being digested into short peptides. The desorbed proteins may also be chemically treated (e.g., reduced) during this step. FIG. 7 Panel G shows the short peptides being analyzed by MALDI mass spectrometry, thus identifying the proteins present in the biomolecule corona formed during this assay.

Some aspects of the present disclosure include a method of assaying a biomolecule, comprising: (a) contacting the biomolecule with a particle, thereby adsorbing the biomolecule to the particle; (b) contacting the biomolecule with a probe comprising (i) a probe and (ii) a barcode, thereby binding the probe binds to the biomolecule; and (c) assaying for the presence of the barcode, to determine the presence of a complex comprising the biomolecule, particle, and probe. In some cases, the biomolecule may be contacted to the particle prior to the probe. In other cases, the biomolecule may be contacted to the probe prior to the particle. In some cases, the biomolecule may be contacted to the particle and the probe in a single step.

The probe may comprise an antibody, a peptide, a nucleic acid ligand (e.g., an aptamer), a Fab, a Fab2, an scFv, an scFab, a nanobody, an aptamer, a polypeptide ligand scaffold, a ligand, or a chemical moiety. The probe may comprise a dimension spanning 1 nm to 35 nm. For example, the probe may comprise a chemical moiety comprising a length of about 1 nm, an IgG antibody comprising a length of about 15 nm, an IgM antibody comprising a diameter of about 35 nm, or a 200 base pair single stranded nucleic acid aptamer comprising a length of about 20 nm when folded. The probe may comprise a molecule mass from about 200 Da to about 200 kDa. In some cases, the probe comprises a peptide comprises an adnectin, an abamer, an affibody, a nanobody, or any combination thereof.

In some cases, the probe is present in a plurality of probes. The plurality of probes may target a plurality of different species. For example, the plurality of probes may comprise a plurality of probes comprising different target affinities. At least a subset of the plurality of probes may comprise a plurality of detection modalities, such as a plurality of different optically detectable dyes. In some cases, at least a subset of the plurality of probes comprise a plurality (e.g., a library) of barcodes. The plurality of barcodes may comprise nucleic acid sequences, peptide sequences, non-biogenic small molecule sequences. In some cases, the plurality of barcodes comprise a plurality of nucleic acid sequences, such that at least a subset of the plurality of detection modalities may uniquely identify at least a subset of the probes, or at least a subset of the plurality of probes coupled thereto. For example, a probe comprising a plurality of probes may comprise a plurality of detection modalities which individual identify the plurality of probes. In some cases, each probe of the plurality of probes comprises a unique barcode, such that each probe may be identified by its barcode. In some cases, the plurality of barcodes comprises from 50 to 10¹⁰ distinct barcodes. In some cases, the library of barcodes comprises a combinatorially generated nucleic acid library. In some cases, the plurality of barcodes comprises nucleotide sequences. In some cases, the assaying comprises measuring a readout indicative of the presence, absence, or amount of the barcode. In some cases, the assaying comprises assaying for the presence or absence of the barcode (e.g., with a hybridization assay).

In some cases, the probe is present in a plurality of probes which bind to different biomolecules and which comprise a plurality of barcodes. In such cases, the plurality of probes may comprise a plurality of probes which bind to the different biomolecules. The plurality of probes may be identified by their barcode sequences. In some cases, a biomolecule to which a probe of the plurality of probes binds may be identified by a barcode coupled to the probe. In some cases, a plurality of biomolecules to which probes of the plurality of probes bind may be identified by the probe barcodes. For example, the presence of an analyte may be determined by identifying the presence of a nucleic acid barcode of a monospecific antibody which targets the analyte.

In some cases, the method is performed under multiple conditions. As a change in condition may alter the solubilities, particle affinities, and inter-biomolecule affinities of biomolecules in a sample, biomolecule corona formation and probe binding can be condition dependent. The composition of a biomolecule corona formed on a particle under a first condition may comprise at most 95%, at most 90%, at most 85%, at most 80%, at most 75%, at most 70%, at most 65%, at most 60%, at most 55%, at most 50%, at most 45%, at most 40%, at most 35%, or at most 30% of biomolecules in common with a biomolecule corona formed on the particle under a second condition upon contact with the same sample. A first condition and a second condition may differ in pH by at least 0.5, at least 1, at least 1.5, at least 2, at least 2.5, or at least 3. A first condition and a second condition may differ in temperature by at least 5° C., at least 10° C., at least 15° C., at least 20° C., or at least 25° C. A first condition and a second condition may differ in viscosity by at least 0.5 centipoise (cP), at least 1 cP, at least 2 cP, at least 5 cP, at least 10 cP, at least 20 cP, at least 30 cP, at least 50 cP, or at least 100 cP. A first condition and a second condition may differ in osmolarity by at least 250 milliosmole (mOsm), at least 500 mOsm, at least 1000 mOsm, at least 2000 mOsm, or at least 3000 mOsm.

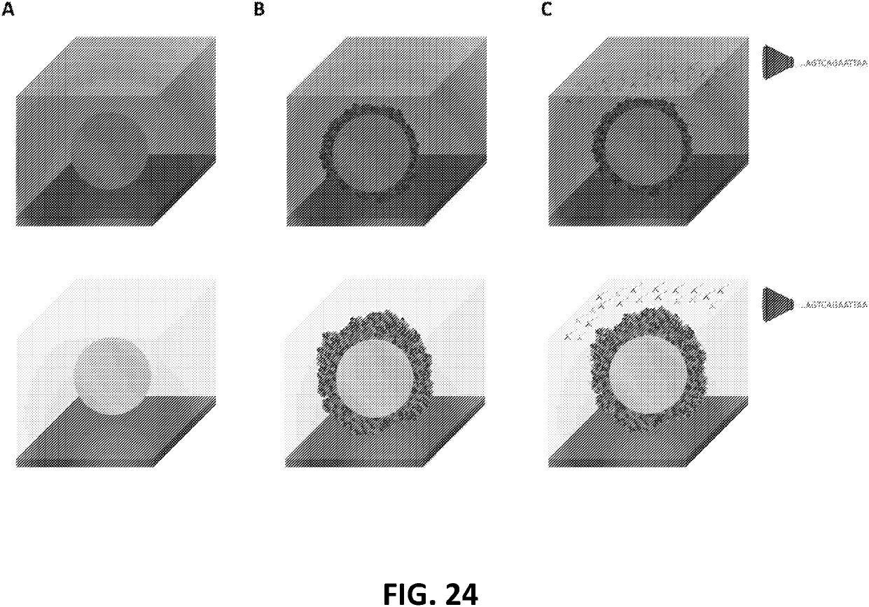

An example of a multi-condition biomolecule corona assay is provided in FIG. 24. This example covers two particles (FIG. 24 Panel A) in separate conditions. For example, the particle in the top row may be in a solution with a relatively high ionic strength of 0.1 mol/kg, a pH of 4.8 and a temperature of 4° C., while the particle in the bottom row may be in a solution with a relatively low ionic strength of 0.005 mol/kg, a pH of 7.1 and a temperature of 31° C. The conditions may be strictly regulated throughout the assay so that pH, ionic strength and temperature remain invariant.

FIG. 24 Panel B shows the particles subsequent to sample contact biomolecule corona formation. The sizes and compositions of the biomolecule coronas differ between particles in the low ionic strength, pH 7.1 solution and the high ionic strength, pH 4.8 solution. FIG. 24 Panel C shows the particles being contacted by a library of probes. The pattern of probe binding may be responsive to solution conditions, which may reflect differences in the biomolecule corona compositions as well as changes in probe binding affinities due to the solution conditions. In this example probe binding profiles are measured by next generation sequencing of probe barcodes. The combination of probe binding profiles between all conditions assayed are used to assign a biomolecule fingerprint to the sample.

In some cases, the assaying for the presence, absence or amount of the probe comprises sequencing the barcode of the probe. The assaying may comprise sequencing at least a portion of the barcode, for example with a next-generation sequencing method such as nanopore sequencing. The assaying may comprise hybridization or affinity capture. For example, the barcode may be contacted to an array comprising complementary nucleic acid sequences in defined an optically resolvable locations and comprising quencher-fluorophore pairs, such that hybridization of the barcode to a complementary nucleic acid sequence may generate a fluorescent signal at a predefined location corresponding to a sequence of the barcode. The assaying may comprise cleaving the barcode from the probe prior to the assaying. As a nonlimiting example, the barcode may be cleaved from the probe while the probe is bound to the biomolecule while the biomolecule is disposed within the biomolecule corona.

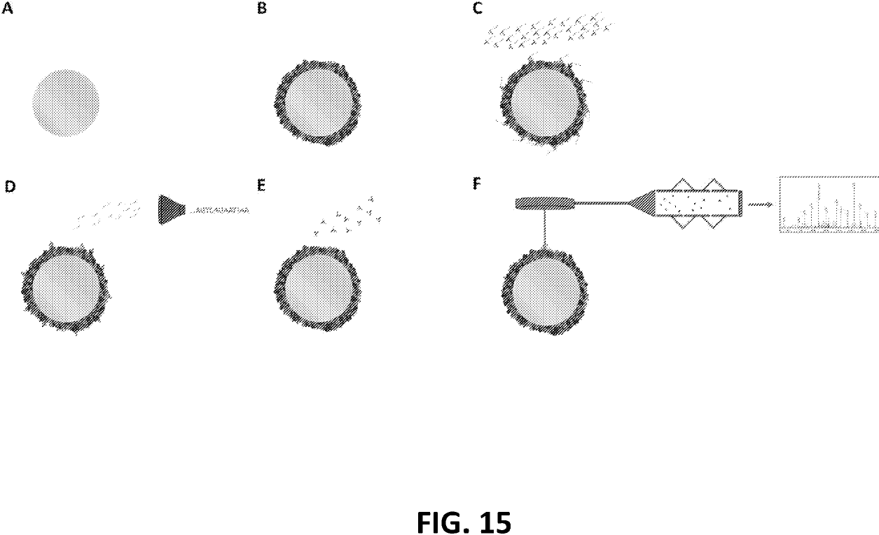

An example of such a method comprising barcode cleavage is illustrated in FIG. 15. In this method, a bare particle may be contacted with a sample under conditions permissive for the adsorption of biomolecules from the sample to the particle, and thereby the formation of a biomolecule corona, as shown in FIG. 15 Panel B. The particle may subsequently be contacted by a library of nucleic acid barcoded probes, as is illustrated in FIG. 15 Panel C, wherein a subset of probes bind to biomolecules on the surface of the biomolecule corona, and the remainder are washed away. Nucleic acid barcodes may be cleaved from the biomolecule corona-bound probes coupled to collection and NGS (FIG. 15 Panel D). Next, FIG. 15 Panel E shows the remaining DEL members being desorbed from the biomolecule corona. FIG. 15 Panel F provides an optional step of biomolecule corona analysis with mass spectrometry.

In some cases, the probe and the barcode are coupled (e.g., conjugated) by a linker. In some cases, the linker comprises a C3 linker, a C6 linker, a C12 linker, a C18 linker, a C36 linker, a peptide linker, a nucleic acid linker, a chemical linker, a PEG linker, or any combination thereof. In some cases, the linker is a cleavable linker. In some cases, the linker is a non-cleavable linker. In some cases, the cleavable linker comprises a protease recognition sequence (e.g., an amino acid sequence towards which the protease comprises cleavage activity). In some cases, the cleavable linker comprises a nuclease recognition sequence.

The probe may be used to assay for inter-biomolecule distances. For example, a first probe of the plurality of probes may comprise a first probe that binds a first biomolecule, and a second probe of the plurality of probes may comprise a second probe that binds a second biomolecule in close proximity with the first biomolecule. In some cases, such tandem binding may enable a barcode of the first probe to hybridize with a barcode of the second probe to generate a hybridized barcode pair. The 3′ ends of the hybridized barcodes may then be extended, thereby encoding a sequence complementary to the barcode of the first probe in the barcode of the second probe, and encoding a sequence complementary to the barcode of the second probe in the barcode of the first probe. Alternatively, the barcode of the first probe and the barcode of the second probe may comprise sticky ends which hybridize and undergo ligation, or blunt ends configured for ligation (e.g., by a T4 DNA ligase). The extended or ligated barcodes may be identified (e.g., sequenced), which may identify the first and second probes from which the barcodes were derived. The extended or ligated barcodes may comprise a primer sequence. The extended or ligated barcodes may be amplified, and the amplicons therefrom may be identified (e.g., sequenced). In some cases, the amplifying comprises thermal cycling amplification, such as polymerase chain reaction amplification (PCR). In some cases, the amplifying comprises isothermal amplification. In some cases, the sequencing comprises next generation sequencing, such as nanopore sequencing.

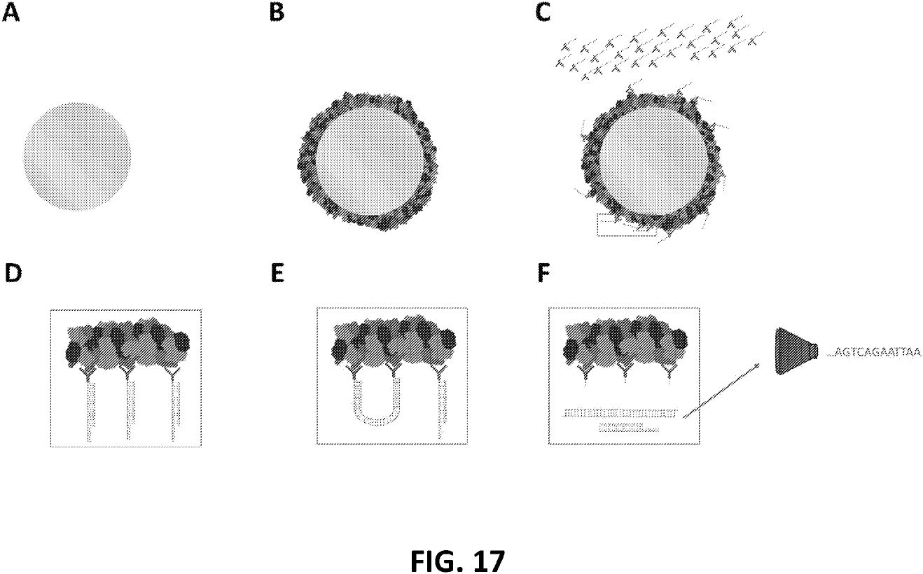

An example of such a distance measurement method is outlined in FIG. 17, which provides a schematic for a proximity ligation assay performed on a biomolecule corona. As shown in FIG. 17 Panels A and B, a particle may be contacted to a sample, thereby forming a biomolecule corona on the particle comprising biomolecules from the sample (FIG. 17 Panel B shows the particle after it has contacted the sample and a biomolecule corona has formed on its surface). As shown in FIG. 17 Panel C, the particle may be subsequently contacted with a library of probes (e.g., a DEL), resulting in a subset of the probes binding to biomolecules on the surface of the biomolecule corona.

FIG. 17 Panel D provides a closeup view of three closely bound probes. Each probes may contain a biomolecule binding portion and a nucleic acid barcode, which may contain an identifier sequence and a sticky end. As is shown in FIG. 17 Panel E, when two probes are bound within sufficient proximity and the sticky ends of their nucleic acid barcodes are sufficiently complementary, their sticky ends may hybridize and may be ligated.

As is shown in FIG. 17 Panel F, the nucleic acid barcodes can be released (e.g., cleaved) from the biomolecule binding portions of the probes and then sequenced. Ligated barcode pairs are read as a single sequence, indicating that the pair of biomolecules targeted by two probes were within a defined proximity. The maximum and minimum distances requisite for nucleic acid barcode hybridization may be a function of probe structure (e.g., the length of a linker coupling a probe to a barcode) and barcode structure (e.g., the lengths and secondary structures of the nucleic acid barcodes). Two probes may be configured to measure a distance of at most 1 nm, at least 1.5 nm, at least 2 nm, at least 2.5 nm, at least 3 nm, at least 4 nm, at least 5 nm, at least 8 nm, at least 10 nm, at least 15 nm, at least 20 nm, at least 25 nm, at least 30 nm, at least 40 nm, or at least 50 nm. Two probes may be configured to measure at distance of at most 50 nm, at most 40 nm, at most 30 nm, at most 25 nm, at most 20 nm, at most 15 nm, at most 10 nm, at most 8 nm, at most 5 nm, at most 4 nm, at most 3 nm, at most 2.5 nm, at most 2 nm, at most 1.5 nm, or at most 1 nm. For example, two probes may be configured to measure a distance of at least 5 nm (e.g., a minimum distance reflective of barcode rigidity and probe size) and at most 15 nm (e.g., a maximum distance reflective of barcode length). Reads of non-ligated barcodes may indicate that a particular biomolecule was present in the sample, and that the biomolecule was not in close proximity to another biomolecular target recognized by the library of probes.

In some cases, the method further comprises performing a wash step after incubating the particle in the sample to wash away biomolecules not adsorbed to the particle. In some cases, the method further comprises performing a wash step after incubating the probe in the sample to wash away unbound probes. In some cases, the method comprises a plurality of wash steps.

The method may comprise contacting the probe with a secondary probe comprising a nucleotide that hybridizes with the barcode. The secondary probe may comprise a detection modality, such as a fluorescent moiety or a mass tag. In some cases, the assaying of c) comprises measuring a readout indicative of the presence, absence, or amount of the detection modality of the secondary probe. In some cases, the secondary probe is present in a plurality of secondary probes comprising different tags and nucleotides that hybridize with different barcode sequences.

The method may further comprise directly analyzing biomolecules of the biomolecule corona. As non-limiting examples, such analyzing may comprise performing mass spectrometry, chromatography, liquid chromatography, high-performance liquid chromatography, solid-phase chromatography, a lateral flow assay, an immunoassay, an enzyme-linked immunosorbent assay, a western blot, a dot blot, or immunostaining, or a combination thereof, on the biomolecule of the biomolecule corona or on one or more other biomolecules of the biomolecule corona.

In some cases, the affinity reagent of the probe comprises the barcode. In some cases, the affinity reagent comprises an aptamer comprising a sequence for its unique identification. In some cases, the affinity reagent and probe each constitute portions of an aptamer.

In some cases, the particle is from about 5 nm to about 50 µm in a dimension. In some cases, the particle is from about 25 nm to about 400 nm in a dimension. In some cases, the particle is from about 50 nm to about 200 nm in a dimension. In some cases, the dimension comprises a diameter. In some cases, the particle comprises an organic, inorganic, hybrid organic-inorganic, or polymeric particle. In some cases, the particle comprises a hollow particle, a solid particle, a porous particle, or a multi-layered particle. In some cases, the particle comprises a sphere, a rod, a triangle, a cylinder, a cube, a low symmetry shape, or another geometrical shape. In some cases, the particle comprises an anionic, cationic, or neutral charge. In some cases, the particle comprises a small surface modification, a peptide surface modification, a protein surface modification, an antibody surface modification, a nucleic acid (e.g., an aptamer) surface modification, a chemical functional group surface modification, or any combination thereof. In some cases, the particle comprises a nanoparticle, microparticle, micelle, liposome, iron oxide particle, graphene particle, silica particle, protein-based particle, polystyrene particle, silver particle, gold particle, quantum dot, palladium particle, platinum particle, titanium particle, or any combinations thereof.

In some cases, the probe comprises a detection modality in addition to or in place of the barcode. In some cases, the detection modality comprises an optically detectable moiety. In some cases, the detection modality comprises a fluorophore. In some cases, the detection modality comprises an electrochemically detectable moiety. In some cases, the detection modality is detectable optically, electrochemically, chemically, magnetically, chromatographically, by affinity capture, or any combination thereof.

In some cases, the method comprises separating the probe from the biomolecule. For example, the method may comprise adding a salt or chaotropic agent to diminish the affinity of the probe for the biomolecule while the biomolecule is disposed within the biomolecule corona, or subsequent to biomolecule elution from the biomolecule corona. The separating may be subsequent to a wash step. For example, the method may comprise removing unbound probes in a wash step, and then separating the probe from the biomolecule. In some cases, the probe is immobilized to a substrate (e.g., a glass slide or a surface of a fluidic chamber) subsequent to separating from the biomolecule. In some cases, the immobilization comprises hybridization of the probe barcode to a capture nucleic acid. In some cases, the immobilization comprises affinity capture (e.g., antibody-based affinity capture). In some cases, the immobilization comprises covalent capture (e.g., click chemistry coupling between a probe-derived azide and a substrate-bound alkyne).

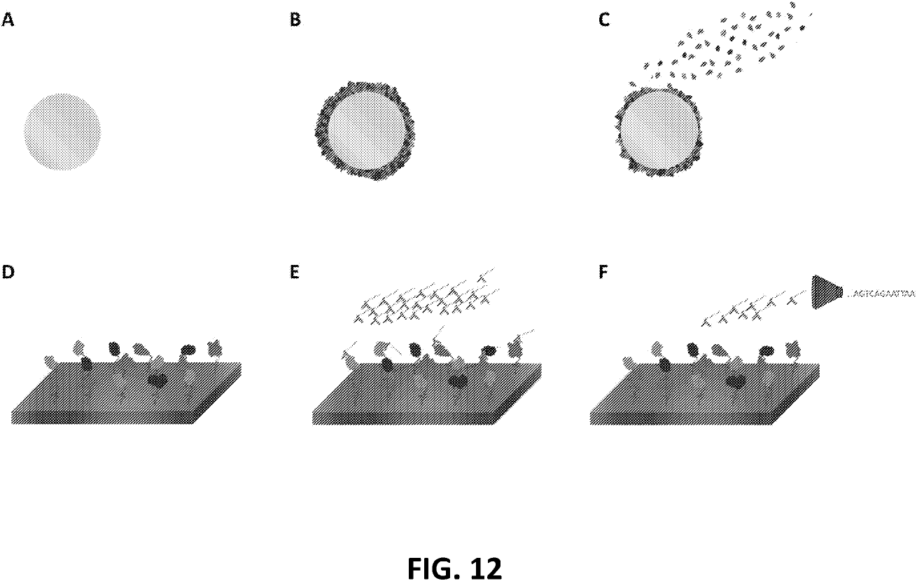

An example of a method comprising elution from a biomolecule corona and subsequent immobilization of biomolecules therefrom is illustrated in FIG. 12, which provides a schematic for a biomolecule analysis method that combines biomolecule corona analysis with biomolecule immobilization and probe-based analysis. FIG. 12 Panel A shows a bare particle prior to contacting a sample. The particle may be contacted with a sample, leading to biomolecule adsorption and formation of a biomolecule corona, as shown in FIG. 12 Panel B. The particle, along with the biomolecule corona adsorbed to the particle, may be separated from unbound biomolecules of the sample, for example through a series of wash steps. Subsequently, biomolecules may be collected from the corona for further analysis. FIG. 12 Panel C shows weakly bound biomolecules (e.g., biomolecules of the soft corona of the particle) desorbing from the biomolecule corona. However, a method may instead comprise elution of most or all biomolecules bound to a particle, or elution of biomolecule corona-bound species through fragmentation, such as digestion. As is shown in FIG. 12 panel D, the desorbed biomolecules may be conjugated to capture moieties bound to a surface, and then contacted by a library of probes, as is shown in FIG. 12 panel E. A subset of the probes may bind to the immobilized biomolecules, while the remainder may be washed away. As shown in FIG. 12 Panel F, probes which bind to the immobilized biomolecules may be analyzed to identify biomolecule or biological state information of the sample. The analysis may comprise elution of the probes from the immobilized biomolecules, for example by a change in pH or addition of a salt or chaotropic agent that lowers the probe affinities for their target biomolecules. Alternatively or in combination, detection modalities (such as fluorophores or barcodes) may be decoupled from the probes to facilitate their detection. Conversely, the probes may be analyzed while bound to their surface immobilized targets. For example, the probes may comprise fluorescent detection modalities which enable fluorescent imaging of the surface comprising the immobilized biomolecules.

In some cases, the biomolecule comprises a protein. In some cases, the protein comprises multiple sites recognized by the affinity reagent. In some cases, the protein comprises a post-translational modification recognizable by the affinity reagent. For example, the protein may comprise a glycosylation pattern recognized by an antibody Fab. In some cases, the biomolecule comprises a lipid, a nucleic acid, or a saccharide (e.g., an oligosaccharide or a polysaccharide).

The sample may comprise a biofluid. In some cases, the biofluid comprises plasma, serum, urine, cerebrospinal fluid, synovial fluid, tears, saliva, whole blood, milk, nipple aspirate, ductal lavage, vaginal fluid, nasal fluid, ear fluid, gastric fluid, pancreatic fluid, trabecular fluid, lung lavage, sweat, crevicular fluid, semen, prostatic fluid, sputum, fecal matter, bronchial lavage, fluid from a swabbing, a bronchial aspirant, or any combination thereof. In some cases, the sample comprises a fluidized solid, a tissue homogenate, a cultured cell, or any combination thereof.

Aspects of the present disclosure provide a method comprising: a) incubating a particle in a biological sample, thereby adsorbing biomolecules from the biological sample onto the particles to form biomolecule coronas; b) incubating the particles with probes comprising (i) affinity reagents and (ii) barcodes, wherein the affinity reagents bind to biomolecules of the biomolecule coronas; c) detecting the presence or amount of the barcodes of the probes comprising affinity reagents bound to biomolecules of the biomolecule coronas; and d) identifying a biomolecule fingerprint associated with the biological sample based on the presence or amount of the barcodes.

In some cases, the method further comprises identifying the presence or amount of the biomolecules of the biomolecule coronas based on the presence or amount of the barcodes. In some cases, identifying the biomolecule fingerprint associated with the biological sample based on the presence or amount of the barcodes comprises identifying the biomolecule fingerprint based on the presence or amount of the biomolecules of the biomolecule coronas. In some cases, the method further comprises identifying a disease state associated with the biomolecule fingerprint. In some cases, the disease state comprises a cancer, cardiovascular disease, endocrine disease, inflammatory disease, or neurological disease. In some cases, identifying the disease state associated with the biomolecule fingerprint comprises applying a classifier to the biomolecule fingerprint. In some cases, the classifier has been trained with data comprising the presence or amounts of barcodes of probes bound to biomolecule coronas of healthy or diseased subjects. In some cases, the particles comprise physiochemically distinct groups of particles.

Aspects of the present disclosure provide a method of assaying a biomolecule in a sample, the method comprising: a) incubating a particle in the sample thereby adsorbing biomolecules from the sample onto the particle to form a biomolecule corona; b) incubating the particle with a probe comprising an affinity reagent that binds to a biomolecule of the biomolecule corona; and c) assaying for the presence, absence or amount of the probe, thereby assaying for the presence, absence or amount of the biomolecule of the biomolecule corona. In some cases, the probe comprises a detection modality. In some cases, the detection modality is detectable optically, electrochemically, chemically, magnetically, chromatographically, by affinity capture, or any combination thereof. In some cases, the detection modality comprises a dye, a fluorescent tag, an electrochemically detectable tag, a magnetic tag, an affinity label, a polymer, a mass tag, or any combination thereof. In some cases, the probe is present in a plurality of probes.

Various aspects of the present disclosure provide a method of assaying a biomolecule in a sample, the method comprising: a) incubating a particle in the sample thereby adsorbing biomolecules from the sample onto the particle to form a biomolecule corona; b) incubating the particle with an affinity reagent that binds to a biomolecule of the biomolecule corona; and c) assaying for the presence, absence or amount of the affinity reagent, thereby assaying for the presence, absence or amount of the biomolecule of the biomolecule corona. In some cases, the affinity reagent comprises a nucleic acid, such as an aptamer. In some cases, the assaying for the presence, absence or amount of the affinity reagent comprises sequencing the nucleic acid. In some cases, the assaying for the presence, absence or amount of the affinity reagent comprises sequencing the aptamer. In some cases, the aptamer binds comprises binding specificity for the biomolecule.

In some cases, the presence, absence, or amount of the biomolecule in the biomolecule corona is indicative of a biological state. In some cases, the biomolecule is more abundant in a sample of a subject having a first biological state than in a sample of a subject having a second biological state. In some cases, the first biological state is an earlier stage of the second biological state. For example, in some cases, the first biological state comprises a stage zero or a stage one cancer, a pre-Alzheimer’s disease, or an early phase of diabetes.

In some cases, the affinity reagent has been evolved to modify its affinity for the biomolecule. For example, the affinity reagent may be subjected to guided or directed evolution to increase its affinity for the biomolecule, or to increase or decrease its affinity for other biomolecules. In the case of a nucleic acid-containing affinity reagent, the affinity reagent may be subjected to error prone nucleic acid amplification to evolve its affinity for the biomolecule and for other targets.

Various aspects of the present disclosure provide a method of assaying a biomolecule in a sample, the method comprising: a) incubating a particle in the sample thereby adsorbing biomolecules from the sample onto the particle to form a biomolecule corona; b) desorbing biomolecules of the biomolecule corona from the particle; c) contacting the desorbed biomolecules with a probe comprising (i) an affinity reagent and (ii) a detection modality, wherein the affinity reagent binds to a biomolecule of the desorbed biomolecules; and d) assaying for the presence, absence or amount of the detection modality of the probe comprising the affinity reagent, thereby assaying for the presence, absence or amount of the biomolecule of the desorbed biomolecules.

In some cases, the method further comprises immobilizing the desorbed biomolecules onto a substrate. The immobilizing may comprise covalent capture, such as maleimide-based N-terminal amine or carbodiimide-based C-terminal carboxylate capture of peptidic biomolecules, nucleophilic phosphate transesterification of nucleic acid biomolecules, or affinity capture of select (e.g., tagged or structurally related) biomolecules. In some cases, the biomolecules are immobilized directly to the substrate. In other cases, the biomolecules are immobilized to the substrate via capture moieties. In some cases, the probe is coupled to the substrate. In some cases, the method further comprises releasing the desorbed biomolecules from the substrate. In some cases, assaying for the presence, absence or amount of the detection modality of the probe comprises assaying for the presence, absence or amount of the detection modality of the probe comprising the affinity reagent bound to the biomolecule of the desorbed biomolecules.

Some aspects of the present disclosure provide an assay method, comprising: a) incubating a particle in a sample, thereby adsorbing biomolecules from the sample onto the particle to form a biomolecule corona; b)incubating the biomolecules of the biomolecule corona with a substrate of a biomolecule of the biomolecule corona; and c) measuring a reaction product of the substrate, thereby assaying for a presence, absence, or an amount of the biomolecule of the biomolecule corona.In some cases, the assay method further comprising incubating the particle with a probe comprising an affinity reagent that binds to the biomolecule of the biomolecule corona, and blocks formation of the reaction product from the substrate.

In some cases, the substrate comprises a flat surface. In some cases, the substrate comprises a particle. In some cases, the substrate comprises glass, a polymer, rubber, plastic, or a metal. In some cases, the substrate comprises a surface of a fluidic chamber, such as a surface of a compartment or channel in a microfluidic device. In some cases, the probe further comprises a barcode nucleotide sequence. In some cases, the assay method further comprises sequencing the barcode. In some cases, the assay method further comprises affinity reagent as an inhibitor of an enzyme activity of the biomolecule, based on the sequencing of the barcode.

Various aspects of the present disclosure provide an assay method, comprising: a) flowing a sample over or through a matrix, thereby adsorbing biomolecules from the sample onto the matrix; b) flowing a probe over or through the matrix, wherein the probe comprises (i) an affinity reagent and (ii) a barcode, and wherein the affinity reagent binds to a biomolecule of the adsorbed biomolecules; and c) assaying for the presence, absence or amount of the probe, thereby assaying for the presence, absence or amount of the biomolecule of the adsorbed biomolecules. In some cases, the matrix is semipermeable. In some cases, the matrix comprises a porous material. In some cases, the matrix comprises a property comprising a charge, a hydrophobicity, or a surface functionalization.

In some aspects, the present disclosure provides a plurality of methods for identifying affinity reagents. In some cases, an affinity reagent will comprise or be coupled to a detection modality that can be used to identify the affinity reagent from among a plurality of affinity reagents. The detection modality may enable quantification of the affinity reagent. In such cases, the detection modality may be used to quantify the amount of a particular type of affinity reagent present within a portion of a sample, such as the amount of a type of affinity reagent bound to the corona of a particular particle. The detection modality may be detected by a range of methods, including optical methods such as fluorescence, fluorescence polarization, FRET, excitation lifetime measurements, phosphorescence, luminescence and absorbance; electrochemical methods such as potentiometry, amperometry, and redox activity; chemical methods such as selective coupling to a capture reagent; and mass spectrometric methods (e.g., the label may have a unique mass spectrometric or tandem MS/MS fingerprint); chromatographic methods; and electrophoretic methods (e.g., gel electrophoresis).

A detection modality may comprise a nucleic acid barcoding sequence. The nucleic acid barcoding sequence may categorize (e.g., identifies an affinity reagent as belonging to a subtype of a plurality of affinity reagents) or uniquely identify an affinity reagent to which it is coupled. In such cases, the affinity reagent may be categorized or identified by sequencing the nucleic acid barcode sequence. A nucleic acid barcoding sequence may comprise dsDNA. A nucleic acid barcoding sequence may comprise ssDNA. A nucleic acid barcoding sequence may comprise RNA. A nucleic acid barcoding sequence may comprise modified or non-natural nucleotides. A nucleic acid barcoding sequence may comprise non-nucleotide molecules.

A nucleic acid barcode may be sequenced. A number of sequencing methods can be used in such an endeavor, including single-molecule real-time sequencing, nanopore ion semiconductor sequencing, pyrosequencing, sequencing by synthesis, sequencing by ligation, and chain termination sequencing. A nucleic acid barcode may also be identified in a hybridization assay (e.g., a fluorescence in situ hybridization assay). Sequencing or identifying a nucleic acid or portion of a nucleic acid that is part of an affinity reagent may not require separating the nucleic acid from the affinity reagent. Sequencing or identifying a nucleic acid that is part of an affinity reagent may not require separating the affinity reagent from a sample. For example, nucleic acid sequences from affinity reagents bound to a sample may be amplified, optionally collected, and sequenced.

The ability to uniquely identify individual affinity reagents from an affinity reagent library can be exploited to gain information on a sample. An assay can involve flowing an affinity reagent library through a sample of biomolecules (e.g., a plurality of biomolecule coronas coupled to a plurality of nanoparticles), removing unbound affinity reagents from the sample, collecting affinity reagents bound to biomolecules in the sample, and determining the identity of each collected affinity reagent. In some cases, the pattern of affinity reagents found to bind to a particular sample can be correlated with particular disease states. In some cases, this can be done without no information or partial information on the identities of the targets of the collected affinity reagents. For example, the patterns of affinity reagents that bind to different biological samples (e.g., two disease states) may be used to train a computing device to use affinity reagent binding to identify unknown biological samples.

An aspect of the present invention are methods involving multi-stage analysis of a sample. For example, an assay may involve binding biomolecules from a sample to an array of nanoparticles to form biomolecule coronas, washing or removing unbound biomolecules, contacting the array with a library of affinity reagents that with certain affinity reagents that can bind to biomolecules within the biomolecule coronas, removing unbound affinity reagents, sequencing nucleic acid barcodes from each affinity reagent bound to a biomolecule from the biomolecule coronas, unbinding affinity reagents from the biomolecule coronas, removing the affinity reagents, and subjecting the biomolecule coronas to further analysis (e.g., mass spectrometric analysis), thereby obtaining affinity reagent binding data and mass spectrometric data on the sample.

In some cases, a probe binding to a sample of biomolecules can be used to determine the types and amounts of biomolecules present in a sample. This can be achieved by predetermining the target specificities of a probes from a probe library. This can also be achieved by calibrating a probe binding results to other forms of data collected in parallel.

Probe binding may be used to determine the proximity of two or more target species. In some cases, the probes of the present invention may be used for Olink assays. For example, any of the compositions or methods disclosed herein may use an Olink detection system as described in U.S. Pat. No. 8,268,554 and U.S. Pat. No. 7,306,904, both of which are herein incorporated by reference in their entirety. A probe library may comprise two a probes comprising complementary nucleic acid sequences. When these two a probes are sufficiently closely spaced (e.g., if the two probes bind proximal targets) the complementary nucleic acid sequences can hybridize. In some cases, the hybridized nucleic acids can undergo extension (e.g. by DNA or RNA polymerase). In some cases, hybridization of the nucleic acids allows hybridization of a template nucleic acid strand. In some cases, the template nucleic acid strand can be extended, amplified (e.g., by rolling circle amplification), sequenced, detected, or any combination thereof. In some cases, the template nucleic acid strand is coupled to a third probe, so that hybridization, amplification, sequencing, and detection require that all three probes bind targets with defined proximities. Such a technique may be used to measure a distance between two or more probe targets. Two probes with complementary nucleic acid sequences may have a maximum distance inter-probe distance at which hybridization can occur. A probe library may have multiple pairs of probes with complementary nucleic acid sequences that bind to the same pairs of targets. Application of the probe library to a sample, followed by amplification and detection, can be used to determine the distance between two targets, as only probe pairs with maximum hybridization distances greater than or equal to the distance between targets within a sample will be detected.

In some cases, the distance is a maximum distance. A pair of probes may comprise a maximum distance over which they will generate a detectable species or signal (e.g., an amplicon or a fluorescent complex). The pair of probes may identify a maximum distance of at most 1 nanometer (nm), at most 1.5 nm, at most 2 nm, at most 2.5 nm, at most 3 nm, at most 4 nm, at most 5 nm, at most 6 nm, at most 8 nm, at most 10 nm, at most 12 nm, at most 15 nm, at most 20 nm, at most 25 nm, at most 30 nm, at most 40 nm, at most 50 nm, at most 60 nm, at most 80 nm, at most 100 nm, at most 120 nm, at most 150 nm, at most 200 nm, or greater than 200 nm. A relatively short maximum distance (e.g., a distance of at most 5 nm) may enable detection of biomolecule-biomolecule interactions (e.g., two biomolecules, such as proteins, are covalently or non-covalently associated), supramolecular complex formation (e.g., two discrete subunits of a multiprotein complex are associated), ligand, substrate, or cofactor binding (e.g., a flavin cofactor is bound to a flavin-dependent enzyme, or a saccharide is bound by an inactive saccharide oxidase). A maximum distance measurement may also determine whether two species are present within a biomolecule corona of a same particle or in biomolecule coronas of different particles. A method may utilize a plurality of probe pairs which identify a plurality of interspecies distances or maximum interspecies-distances. Such a method may generate different identifiable signals for different probe pairs, such as a first amplicon for 5 nm distances, a second amplicon for 10 nm distances, and a third amplicon for 20 nm distances.

Such a technique can be applied to determine whether a biomolecule (e.g., a protein) has a particular post-translational modification. For example, a probe library may comprise two probes that, i) target two sites on the same protein and, ii) comprise complementary nucleic acid sequences. One of the probes may target a glycosylation pattern that is sometimes present on the protein. When the protein is glycosylated at the target site, the two probes will bind to the protein, allowing their complementary nucleic acid sequences to hybridize and be detected.

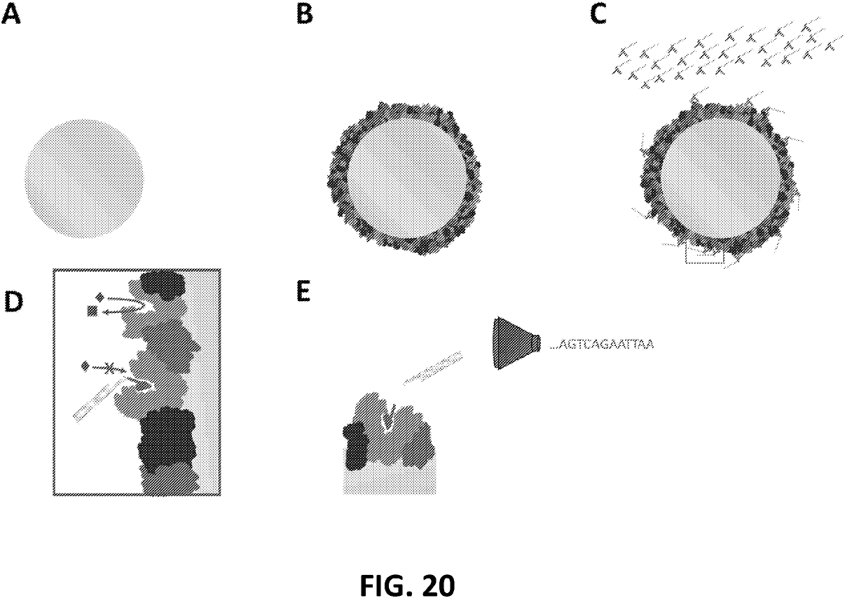

Various aspects of the present disclosure provide methods for identifying protein agonists, protein antagonists, protein cofactors, enzyme substrate, enzyme inhibitors, enzyme activity, or any combination thereof. FIG. 20 provides an example of a method for identifying enzyme inhibitors or elucidating enzyme activity by interrogating probe binding to a biomolecule corona. FIG. 20 Panel A shows a bare particle prior to contact with a sample. FIG. 20 Panel B shows the particle following biomolecule corona formation following contact with the sample. FIG. 20 Panel C shows the particle subsequently being contacted by a library probes comprising nucleic acid barcodes, wherein a subset of probes bind to biomolecules on the surface of the biomolecule corona. The particle is then contacted with a substrate of an enzyme present in the sample (FIG. 20 Panel D). The rate of the reaction can be monitored mass spectrometrically, spectroscopically, electrochemically, colorimetrically, chromatographically, or any combination thereof. The presence of an enzyme inhibitor in the probe library can be detected as a reduction in enzymatic activity. The identity of the inhibitory affinity binding reagent can be determined by performing multiple parallel reactions with partially overlapping probe libraries. This type of assay may be incorporated into other types of assays, including Proteograph, to further elucidate a biological state. For example, diseases caused by constitutively activated ubiquitin ligases could be identified by parallel Proteograph and ubiquitin ligase activity assays.

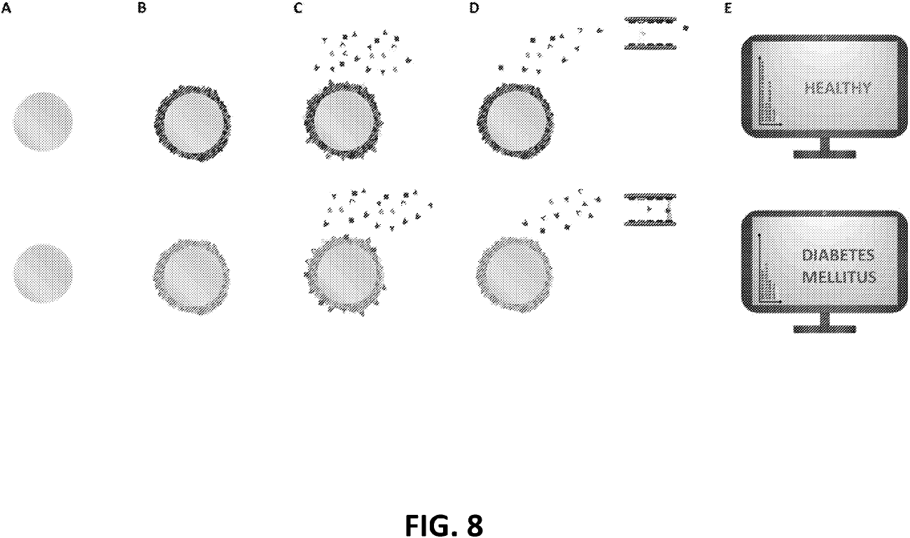

FIG. 21 illustrates a probe library evolution method that utilizes biomolecule corona analysis. A combinatorial library of polynucleotides is randomly assembled from small nucleic acid library comprising a number of short nucleic acid sequences (panel A). The polynucleotide library is then contacted with a set of oligonucleotides coupled to reactive groups (panel B). If the sequence of a reactive-group bearing oligonucleotide is present in a polynucleotide from the combinatorial library, the two species will hybridize, and the reactive group will transfer from the oligonucleotide to the polynucleotide. Multiple contacting rounds may be used to generate complex sequences of reactive groups on each polynucleotide. As is shown in panel C, the library of reactive group-bearing polynucleotides is then contacted to a particle covered with a biomolecule corona. A subset of polynucleotides will be coupled to sequences of reactive groups with affinities for a corona-bound biomolecule. The remaining polynucleotides will be washed away. The remaining nucleotides can optionally be digested, amplified, reassembled to form a new polynucleotide library, and subjected to additional rounds of evolution. This library evolution scheme can be used to generate probes with specificity for a particular biomolecule (e.g., ceruloplasmin) or disease state (e.g., Wilson’s disease). This method can also be used to generate a library with a plurality of probes targeting a plurality of biomolecules. This method can also be coupled to the method outlined in FIG. 8 to identify inhibitors for a particular enzyme.

In some cases, two probes may have nucleic acid sequences configured for recombination or ligation. In such cases, recombination or ligation of the nucleic acid sequences may require or be affected by the two probes binding to targets within a defined proximity (e.g., a distance defined by the lengths of the two probes). In some cases, ligation or recombination of the two nucleic acid sequences can allow hybridization of a template strand. In some cases, ligated or recombinantly modified nucleic acid sequences can undergo amplification, sequencing, detection, or any combination thereof. In some cases, ligation or recombination of the two nucleic acid sequences can be used to determine the proximity of two or more targets (e.g., two proteins, or a protein and cofactor).

In some cases, the sample is analyzed with probe binding and mass spectrometry. In some cases, the sample may be collected on a particle or particle array prior to probe and MS analysis. In some cases, a sample is first interrogated with a probe binding assay, and then subsequently analyzed with MS. In some cases, a biomolecule corona may be desorbed from the particle prior to MS analysis. In some cases, a biomolecule corona may be desorbed from a particle prior to a probe binding assay. Advantageously, the methods disclosed herein do not require mass spectrometry.

In some cases, the probe binding assay and MS measure complimentary portions of the sample. For example, a probe binding assay may detect small molecules in a sample, and MS may detect proteins in a sample. In some cases, separate detection of small molecules and proteins in a sample may be used to identify cofactors or substrates for a particular protein.

In some cases, tandem MS-probe binding assays may be used to determine a protein’s conformational state or post-translational modifications. This can be achieved by first contacting a sample with a probe that binds a particular conformational state or set of post-translational modifications of the protein, assaying for probe binding, and then performing MS to determine whether the protein is present. A positive result from the probe binding assay will indicate that the protein is present and is in the conformation or comprises the post-translational modifications required for the probe to bind the protein. A negative result for probe binding coupled with MS detection of the protein will indicate that the protein is not in the conformational state or does not comprise the post-translational modifications required for the probe to bind to the protein.

In some cases, a probe library contains a plurality of probes that are optically detectable (e.g., by fluorescence). In some cases, a biomolecule corona may be interrogated with a probe binding assay comprising optical (e.g., fluorescence) detection of probes bound to a biomolecule corona. In some cases, the probes are sufficiently small to allow complete coverage of the species on the surface of the biomolecule corona. In some cases, probes bind to a subset of the species on the surface of the corona. In some cases, probes bind to species that are below the surface of the corona.

In some cases, a probe library contains a probe comprising a fluorophore and a probe comprising a quencher for the fluorophore. In some cases, a probe library contains a probe comprising a FRET donor and a probe comprising a FRET acceptor. In some cases, a fluorescence signal from a probe binding assay may provide information on the relative proximity of two species (e.g., a first probe binding target and a second probe binding target). In some cases, both probe binding targets are localized within a particular biomolecule corona.

In various aspects, the present disclosure provides a method of assaying a biomolecule in a sample, the method comprising: incubating a sample with a probe comprising an affinity reagent, thereby binding the affinity reagent to the biomolecule; and assaying for the probe, thereby assaying for the biomolecule. Assaying for the probe may include assaying for the probe bound to the affinity reagent.

### Biomolecule Corona Analysis With Probes

Aspects of the present disclosure provide methods for analyzing complex biological samples with a particle and a probe. Probe (e.g., aptamer) analysis is often challenged by off-target binding and limited probe detection efficiencies, and is therefore often limited to high-specificity probes (e.g., monospecific probes) and purified samples (e.g., albumin and globulin depleted plasma). For example, a probe comprising picomolar affinities for a range of nanomolar plasma-derived signaling molecules and a weaker, millimolar affinity for albumin may be sequestered by albumin when contacted to a plasma sample due to its high concentration of albumin. Pre-fractionating a sample through biomolecule corona formation can circumvent this issue by enriching low abundance biomolecules from a sample, in some cases also diminishing the sample’s dynamic range.

Biomolecule corona formation can also diminish the number of analytes present for analysis. For example, a plasma sample comprising over 5000 types of proteins may be too complex for some probe analysis methods, as each probe may comprise non-negligible affinities for hundreds or thousands of proteins, and thus produce indeterminate or non-concrete data. Conversely, a biomolecule corona generated from a plasma sample may comprise 10-500 proteins, thereby narrowing the range of interactions between each probe and the sample analytes. In many cases, the subset of biomolecules collected on a particle may be of greater relevance for a biological state determination. Often, the subset of biomolecules collected on a particle comprise an increased proportion of low abundance biomolecules, whose presence and relative abundances may provide more information than high abundance biomolecules from the sample.

In some cases, the contacting and the analysis are performed in a single device, vessel or compartment. In some cases, the analyzing does not comprise analyzing the sample with mass spectrometry. For example, a device may be configured to collect a subset of molecules from a sample on an array of particles (e.g., by forming biomolecule coronas on the particles), contact the array with a probe library (e.g., a DNA encoded library), remove unbound probes, and elute target molecule-bound probes into a second compartment for next-generation sequencing (NGS). In some cases, multiple cycles of probe binding and analysis may be performed on a single sample.

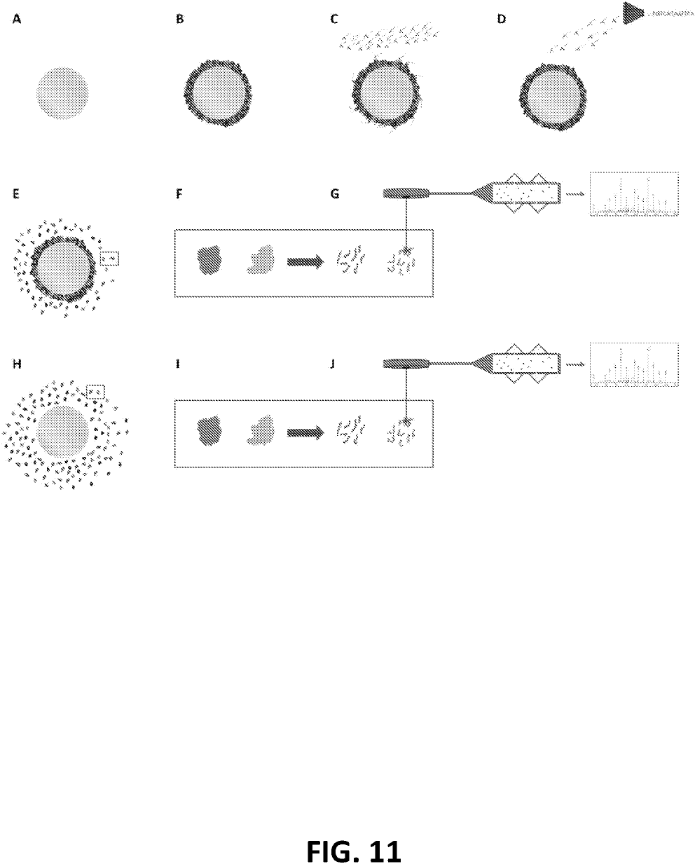

FIG. 11 shows a schematic for a proteome analysis method that combines biomolecule corona analysis with a probe (e.g., a DNA encoded library (DEL)) binding assay. FIG. 11 Panel A shows a bare particle prior to contacting a sample. FIG. 11 Panel B shows the particle following contact with a sample and formation of a biomolecule corona. FIG. 11 Panel C shows the particle subsequently being contacted by a library of probes, wherein a subset of members of the library of probes bind to biomolecules on the surface of the biomolecule corona, and the remainder are washed away. FIG. 11 Panel D shows the bound probes being desorbed from the corona and identified by NGS. The NGS can determine the identities and relative or absolute quantities of each ligand present.

FIG. 11 Panels E-J show optional steps involving mass spectrometric analysis of the biomolecule corona. FIG. 11 Panel E shows the soft corona portion of the biomolecule corona being desorbed into solution. FIG. 11 Panel F shows desorbed proteins being digested into short peptides. Panel G shows the short peptides being analyzed by MALDI mass spectrometry. Panel H shows the hard biomolecule corona being desorbed from the particle. Panel I shows desorbed proteins being digested into short peptides. FIG. 11 Panel J shows the short peptides being analyzed by MALDI mass spectrometry. Thus, this assay can distinguish and independently identify biomolecules with different affinities for a particular particle’s biomolecule corona. In some cases, a particular biomolecule’s affinity for biomolecule corona binding may be dependent on the biological state associated with the sample. For example, a disease may lead to raised cell free DNA concentrations, which in turn may lower a particular protein’s affinity for binding to biomolecule coronas formed from that sample.

### Kits

Provided herein are kits comprising compositions of the present disclosure that may be used to perform the methods of the present disclosure. A kit may comprise one or more particle types to interrogate a sample to identify a biological state of a sample. In some cases, a kit may comprise a particle type provided in TABLE 1. A kit may comprise a reagent for functionalizing a particle (e.g., a reagent for tethering a small molecule functionalization to a particle surface). The kit may be pre-packaged in discrete aliquots. In some cases, the kit can comprise a plurality of different particle types that can be used to interrogate a sample. The plurality of particle types can be pre-packaged where each particle type of the plurality is packaged separately. Alternately, the plurality of particle types can be packaged together to contain combination of particle types in a single package. A particle may be provided in dried (e.g., lyophilized) form, or may be provided in a suspension or solution. The particles may be provided in a well plate. For example, a kit may contain an 8 well plate, an 8-384 well plate with particles provided (e.g., sealed) within the wells. For example, a well plate may comprise at least 8, at least 16, at least 24, at least 32, at least 40, at least 48, at least 56, at least 64, at least 72, at least 80, at least 88, at least 96, at least 104, at least 112, at least 120, at least 128, at least 136, at least 144, at least 152, at least 160, at least 168, at least 176, at least 184, at least 192, at least 200, at least 208, at least 216, at least 224, at least 232, at least 240, at least 248, at least 256, at least 264, at least 272, at least 280, at least 288, at least 296, at least 304, at least 312, at least 320, at least 328, at least 336, at least 344, at least 352, at least 360, at least 368, at least 376, at least 384, at least 392, at least 400 wells comprising particles. Two wells in such a well plate may contain different particles or different concentrations of particles. Two wells may comprise different buffers or chemical conditions. For example, a well plate may be provided with different particles in each row of wells and different buffers in each column of rows. A well may be sealed by a removable covering. For example, a kit may comprise a well plate comprising a slip covering a plurality of wells (e.g., a plastic coverslip). A well may be sealed by a pierceable covering. For example, a well may be covered by a septum that a needle can pierce to facilitate sample movement into and out of the well.

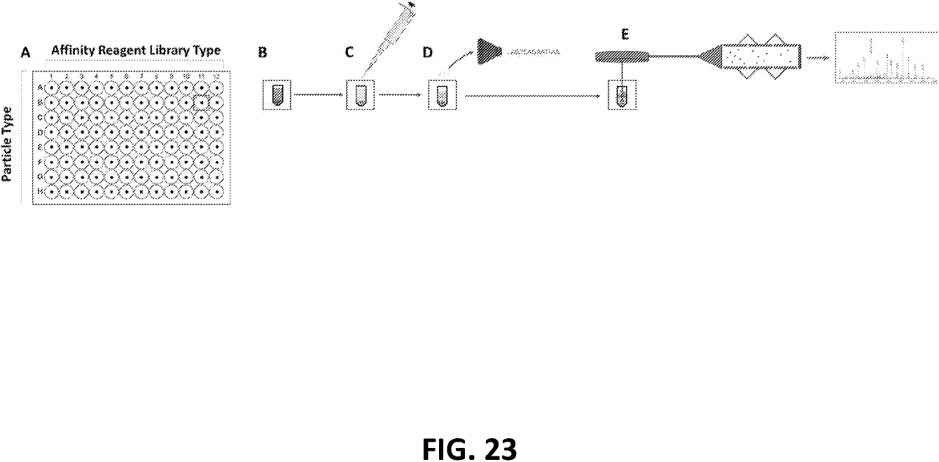

FIG. 23 illustrates a well plate consistent with the present disclosure, as well as a method for using the well plate to assay a sample. The well plate may be loaded with different combinations of samples, sensor elements (e.g., particles), probes, or any combination thereof. For example, as illustrated in FIG. 23 Panel A, each well may correspond to a distinct particle-probe (or probe library) combination. The well plate may comprise particles. The particles may be in solution or in dried form. The particles may optionally be rehydrated and then contacted with sample, as shown in FIG. 23 Panel B, to form biomolecule coronas on the particles. The contents of each well can then undergo multiple washes (FIG. 23 Panel C), removing non-particle-bound species, and leaving the biomolecule corona-coated particles. This step may be performed with a filter-tipped aspirator configured to prevent particle removal from each well, by magnetic particle sequestration, by particle immobilization (e.g., the particles may be provided coupled to surfaces of the wells), or any combination thereof. Next, the biomolecule coronas of each well may be interrogated by a probe or probe library (FIG. 23 Panel D). The nucleic acid barcodes can then be collected and sequenced. The biomolecules in each well may then optionally be analyzed by (for example by mass spectrometry, as shown in FIG. 23 Panel E). Each step may be automated, and multiple steps may be performed in parallel. For example, the multi-well plate could be loaded into a device that performs each well assay in parallel. Alternatively, the contents of each well may be individually aspirated into separate containers (e.g., a spin down column) for analysis.

### Samples

The present disclosure provides a range of samples that can be assayed using the particles and the methods provided herein. A sample may be a biological sample (e.g., a sample derived from a living organism). A sample may comprise a cell or be cell-free. A sample may comprise a biofluid, such as blood, serum, plasma, urine, or cerebrospinal fluid (CSF). Samples consistent with the present disclosure include biological samples from a subject. The subject may be a human or a non-human animal. Said biological samples can contain a plurality of proteins or proteomic data, which may be analyzed after adsorption of proteins to the surface of the various sensor element (e.g., particle) types in a panel and subsequent digestion of protein coronas. Proteomic data can comprise nucleic acids, peptides, or proteins. A biofluid may be a fluidized solid, for example a tissue homogenate, or a fluid extracted from a biological sample. A biological sample may be, for example, a tissue sample or a fine needle aspiration (FNA) sample. A biological sample may be a cell culture sample. For example, a biofluid may be a fluidized cell culture extract.

A wide range of samples are compatible for use within the methods and compositions of the present disclosure. The biological sample may comprise plasma, serum, urine, cerebrospinal fluid, synovial fluid, tears, saliva, whole blood, milk, nipple aspirate, ductal lavage, vaginal fluid, nasal fluid, ear fluid, gastric fluid, pancreatic fluid, trabecular fluid, lung lavage, sweat, crevicular fluid, semen, prostatic fluid, sputum, fecal matter, bronchial lavage, fluid from swabbings, bronchial aspirants, fluidized solids, fine needle aspiration samples, tissue homogenates, lymphatic fluid, cell culture samples, or any combination thereof. The biological sample may comprise multiple biological samples (e.g., pooled plasma from multiple subjects, or multiple tissue samples from a single subject). The biological sample may comprise a single type of biofluid or biomaterial from a single source.

The biological sample may be diluted or pre-treated. The biological sample may undergo depletion (e.g., the biological sample comprises serum) prior to or following contact with a particle or plurality of particles. The biological sample may also undergo physical (e.g., homogenization or sonication) or chemical treatment prior to or following contact with a particle or plurality of particles. The biological sample may be diluted prior to or following contact with a particle or plurality of particles. The dilution medium may comprise buffer or salts, or be purified water (e.g., distilled water). Different partitions of a biological sample may undergo different degrees of dilution. A biological sample or a portion thereof may undergo a 1. 1-fold, 1.2-fold, 1.3-fold, 1.4-fold, 1.5-fold, 2-fold, 3-fold, 4-fold, 5-fold, 6-fold, 8-fold, 10-fold, 12-fold, 15-fold, 20-fold, 30-fold, 40-fold, 50-fold, 75-fold, 100-fold, 200-fold, 500-fold, or 1000-fold dilution.

The compositions and methods of the present disclosure can be used to measure, detect, and identify specific proteins from biological samples. Examples of proteins that can be identified and measured include highly abundant proteins, proteins of medium abundance, and low-abundance proteins. For example, a composition or method may identify at least 1, at least 2, at least 3, at least 4, at least 5, at least 6, at least 7, at least 8, at least 10, at least 12, at least 15, at least 18, at least 20, at least 25, at least 30, at least 35, at least 40, or at least 50 human plasma proteins from the group consisting of albumin, immunoglobulin G (IgG), lysozyme, carcino embryonic antigen (CEA), receptor tyrosine-protein kinase erbB-2 (HER-2/neu), bladder tumor antigen, thyroglobulin, alpha-fetoprotein, prostate specific antigen (PSA), mucin 16 (CA125),carbohydrate antigen 19-9 (CA19.9), carcinoma antigen 15-3 (CA15.3), leptin, prolactin, osteopontin, insulin-like growth factor 2 (IGF-II), 4F2 cell-surface antigen heavy chain (CD98), fascin, sPigR, 14-3-3 eta, troponin I, B-type natriuretic peptide, breast cancer type 1 susceptibility protein (BRCA1), c-Myc proto-oncogene protein (c-Myc), interleukin-6 (IL-6), fibrinogen, epidermal growth factor receptor (EGFR), gastrin, PH, granulocyte colony-stimulating factor (G CSF), desmin, enolase 1 (NSE), folice-stimulating hormone (FSH), vascular endothelial growth factor (VEGF), P21, Proliferating cell nuclear antigen (PCNA), calcitonin, pathogenesis-related proteins (PR), luteinizing hormone (LH), somatostatin S100, insulin. alpha-prolactin, adrenocorticotropic hormone (ACTH), B-cell lymphoma 2 (Bcl 2), estrogen receptor alpha (ER alpha), antigen k (Ki-67), tumor protein (p53), cathepsin D, beta catenin, von Willebrand factor (VWF), CD15, k-ras, caspase 3, ENTH domain-containing protein (EPN), CD 10, FAS, breast cancer type 2 susceptibility protein (BRCA2), CD30L, CD30, CGA, CRP, prothrombin, CD44, APEX, transferrin, GM-CSF, E-cadherin, interleukin-2 (IL-2), Bax, IFN-gamma, beta-2-MG, tumor necrosis factor alpha (TNF alpha), cluster of differentiation 340, trypsin, cyclin D1, MGB, XBP-1, HG-1, YKL-40, S-gamma, ceruloplasmin, NESP-55, netrin-1, geminin, GADD45A, CDK-6, CCL21, breast cancer metastasis suppressor 1 (BrMS1), 17betaHDI, platelet-derived growth factor receptor A (PDGRFA), P300/CBP-associated factor (Pcaf), chemokine ligand 5 (CCL5), matrix metalloproteinase-3 (MMP3), claudin-4, and claudin-3.

Proteins in the biological sample may include a post-translational modification. Some non-limiting examples of post-translational modifications include glycosylation, acetylation, alkylation, biotinylation, glutamylation, glycylation, isoprenylation, phosphorylation, lipolation, phosphopantetheinylation, sulfation, selenation, amidation, ubiquitination, hydroxylation, nitrosylation, or SUMOylation.

Any of the probes, affinity reagents, libraries (e.g., DNA encoded libraries of affinity reagents), particles, and detection modalities disclosed herein can be used for assaying proteins in the corona of said particles after incubation with a wide variety of samples. These materials can be used combinatorically in the methods disclosed herein of rapidly identifying proteins in a sample of interest. Samples consistent with the methods disclosed herein can include biological samples from a subject. The subject may be a human or a non-human animal. Biological samples may be a biofluid. For example, the biofluid may be plasma, serum, CSF, urine, tear, cell lysates, tissue lysates, cell homogenates, tissue homogenates, nipple aspirates, fecal samples, synovial fluid and whole blood, or saliva. Samples can also be non-biological samples, such as water, milk, solvents, or anything homogenized into a fluidic state. Said biological samples can contain a plurality of proteins or proteomic data, which may be analyzed after adsorption of proteins to the surface of the various particle types in a panel and subsequent digestion of protein coronas. Proteomic data can comprise nucleic acids, peptides, or proteins. Any of the samples herein can contain a number of different analytes, which can be analyzed using the compositions and methods disclosed herein. The analytes can be proteins, peptides, small molecules, nucleic acids, metabolites, lipids, or any molecule that could potentially bind or interact with the surface of a particle type.

Disclosed herein are compositions and methods for multi-omic analysis. “Multi-omic(s)” or “multiomic(s)” can refer to an analytical approach for analyzing biomolecules at a large scale, wherein the data sets are multiple omes, such as proteome, genome, transcriptome, lipidome, and metabolome. Non-limiting examples of multi-omic data includes proteomic data, genomic data, lipidomic data, glycomic data, transcriptomic data, or metabolomics data. “Biomolecule” in “biomolecule corona” can refer to any molecule or biological component that can be produced by, or is present in, a biological organism. Non-limiting examples of biomolecules include proteins (protein corona), polypeptides, polysaccharides, a sugar, a lipid, a lipoprotein, a metabolite, an oligonucleotide, a nucleic acid (DNA, RNA, micro RNA, plasmid, single stranded nucleic acid, double stranded nucleic acid), metabolome, as well as small molecules such as primary metabolites, secondary metabolites, and other natural products, or any combination thereof. In some embodiments, the biomolecule is selected from the group of proteins, nucleic acids, lipids, and metabolomes.

### Biological States

The compositions and methods disclosed herein can be used to identify various biological states in a particular biological sample. For example, a biological state can refer to an elevated or low level of a particular protein or a set of proteins. In other examples, a biological state can refer to identification of a disease, such as cancer. The particles, affinity reagents, and methods of us thereof can be used to distinguish between two biological states. The two biological states may be related diseases states (e.g., two HRAS mutant colon cancers or different stages of a type of a cancer). The two biological states may be different phases of a disease, such as pre-Alzheimer’s disease and early-onset Alzheimer’s disease. The two biological states may be distinguished with a high degree of accuracy (e.g., the percentage of accurately identified biological states among a population of samples). For example, the compositions and methods of the present disclosure may distinguish two biological states with at least 60% accuracy, at least 70% accuracy, at least 75% accuracy at least 80% accuracy, at least 85% accuracy, at least 90% accuracy, at least 95% accuracy, at least 98% accuracy, or at least 99% accuracy. The two biological states may be distinguished with a high degree of specificity (e.g., the rate at which negative results are correctly identified among a population of samples). For example, the compositions and methods of the present disclosure may distinguish two biological states with at least 60% specificity, at least 70% specificity, at least 75% specificity at least 80% specificity, at least 85% specificity, at least 90% specificity, at least 95% specificity, at least 98% specificity, or at least 99% specificity.

The methods, compositions, and systems described herein can be used to determine a disease state, and/or prognose or diagnose a disease or disorder. The diseases or disorders contemplated include, but are not limited to, for example, cancer, cardiovascular disease, endocrine disease, inflammatory disease, a neurological disease and the like.

The methods, compositions, and systems described herein can be used to determine, prognose, and/or diagnose a cancer disease state. The term “cancer” is meant to encompass any cancer, neoplastic and preneoplastic disease that is characterized by abnormal growth of cells, including tumors and benign growths. Cancer may, for example, be lung cancer, pancreatic cancer, or skin cancer. In many cases, the methods, compositions and systems described herein are not only able to diagnose cancer (e.g. determine if a subject (a) does not have cancer, (b) is in a pre-cancer development stage, (c) is in early stage of cancer, (d) is in a late stage of cancer) but are able to determine the type of cancer.

The methods, compositions, and systems of the present disclosure can additionally be used to detect other cancers, such as acute lymphoblastic leukemia (ALL); acute myeloid leukemia (AML); cancer in adolescents; adrenocortical carcinoma; childhood adrenocortical carcinoma; unusual cancers of childhood; AIDS-related cancers; kaposi sarcoma (soft tissue sarcoma); AIDS-related lymphoma (lymphoma); primary cns lymphoma (lymphoma); anal cancer; appendix cancer – see gastrointestinal carcinoid tumors; astrocytomas, childhood (brain cancer); atypical teratoid/rhabdoid tumor, childhood, central nervous system (brain cancer); basal cell carcinoma of the skin – see skin cancer; bile duct cancer; bladder cancer; childhood bladder cancer ; bone cancer (includes ewing sarcoma and osteosarcoma and malignant fibrous histiocytoma); brain tumors; breast cancer; childhood breast cancer; bronchial tumors, childhood; burkitt lymphoma – see non-hodgkin lymphoma; carcinoid tumor (gastrointestinal); childhood carcinoid tumors; carcinoma of unknown primary; childhood carcinoma of unknown primary; cardiac (heart) tumors, childhood; central nervous system; atypical teratoid/rhabdoid tumor, childhood (brain cancer); embryonal tumors, childhood (brain cancer); germ cell tumor, childhood (brain cancer); primary cns lymphoma; cervical cancer; childhood cervical cancer; childhood cancers; cancers of childhood, unusual; cholangiocarcinoma – see bile duct cancer; chordoma, childhood; chronic lymphocytic leukemia (CLL); chronic myelogenous leukemia (CML); chronic myeloproliferative neoplasms; colorectal cancer; childhood colorectal cancer; craniopharyngioma, childhood (brain cancer); cutaneous t-cell lymphoma – see lymphoma (mycosis fungoides and sezary syndrome); ductal carcinoma in situ (DCIS) – see breast cancer; embryonal tumors, central nervous system, childhood (brain cancer); endometrial cancer (uterine cancer); ependymoma, childhood (brain cancer); esophageal cancer; childhood esophageal cancer; esthesioneuroblastoma (head and neck cancer); ewing sarcoma (bone cancer); extracranial germ cell tumor, childhood; extragonadal germ cell tumor; eye cancer; childhood intraocular melanoma; intraocular melanoma; retinoblastoma; fallopian tube cancer; fibrous histiocytoma of bone, malignant, and osteosarcoma; gallbladder cancer; gastric (stomach) cancer; childhood gastric (stomach) cancer; gastrointestinal carcinoid tumor; gastrointestinal stromal tumors (GIST) (soft tissue sarcoma); childhood gastrointestinal stromal tumors; germ cell tumors; childhood central nervous system germ cell tumors (brain cancer); childhood extracranial germ cell tumors; extragonadal germ cell tumors; ovarian germ cell tumors; testicular cancer; gestational trophoblastic disease; hairy cell leukemia; head and neck cancer; heart tumors, childhood; hepatocellular (liver) cancer; histiocytosis, langerhans cell; hodgkin lymphoma; hypopharyngeal cancer (head and neck cancer); intraocular melanoma; childhood intraocular melanoma; islet cell tumors, pancreatic neuroendocrine tumors; kaposi sarcoma (soft tissue sarcoma); kidney (renal cell) cancer; langerhans cell histiocytosis; laryngeal cancer (head and neck cancer); leukemia; lip and oral cavity cancer (head and neck cancer); liver cancer; lung cancer (non-small cell and small cell); childhood lung cancer; lymphoma; male breast cancer; malignant fibrous histiocytoma of bone and osteosarcoma; melanoma; childhood melanoma; melanoma, intraocular (eye); childhood intraocular melanoma; merkel cell carcinoma (skin cancer); mesothelioma, malignant; childhood mesothelioma; metastatic cancer; metastatic squamous neck cancer with occult primary (head and neck cancer); midline tract carcinoma with nut gene changes; mouth cancer (head and neck cancer); multiple endocrine neoplasia syndromes; multiple myeloma/plasma cell neoplasms; mycosis fungoides (lymphoma); myelodysplastic syndromes, myelodysplastic/myeloproliferative neoplasms; myelogenous leukemia, chronic (cml); myeloid leukemia, acute (aml); myeloproliferative neoplasms, chronic; nasal cavity and paranasal sinus cancer (head and neck cancer); nasopharyngeal cancer (head and neck cancer); neuroblastoma; non-hodgkin lymphoma; non-small cell lung cancer; oral cancer, lip and oral cavity cancer and oropharyngeal cancer (head and neck cancer); osteosarcoma and malignant fibrous histiocytoma of bone; ovarian cancer; childhood ovarian cancer; pancreatic cancer; childhood pancreatic cancer; pancreatic neuroendocrine tumors (islet cell tumors); papillomatosis (childhood laryngeal); paraganglioma; childhood paraganglioma; paranasal sinus and nasal cavity cancer (head and neck cancer); parathyroid cancer; penile cancer; pharyngeal cancer (head and neck cancer); pheochromocytoma; childhood pheochromocytoma; pituitary tumor; plasma cell neoplasm/multiple myeloma; pleuropulmonary blastoma; pregnancy and breast cancer; primary central nervous system (CNS) lymphoma; primary peritoneal cancer; prostate cancer; rectal cancer; recurrent cancer; renal cell (kidney) cancer; retinoblastoma; rhabdomyosarcoma, childhood (soft tissue sarcoma); salivary gland cancer (head and neck cancer); sarcoma; childhood rhabdomyosarcoma (soft tissue sarcoma); childhood vascular tumors (soft tissue sarcoma); ewing sarcoma (bone cancer); kaposi sarcoma (soft tissue sarcoma); osteosarcoma (bone cancer); soft tissue sarcoma; uterine sarcoma; sezary syndrome (lymphoma); skin cancer; childhood skin cancer; small cell lung cancer; small intestine cancer; soft tissue sarcoma; squamous cell carcinoma of the skin - see skin cancer; squamous neck cancer with occult primary, metastatic (head and neck cancer); stomach (gastric) cancer; childhood stomach (gastric) cancer; t-cell lymphoma, cutaneous - see lymphoma (mycosis fungoides and sezary syndrome); testicular cancer; childhood testicular cancer; throat cancer (head and neck cancer); nasopharyngeal cancer; oropharyngeal cancer; hypopharyngeal cancer; thymoma and thymic carcinoma; thyroid cancer; transitional cell cancer of the renal pelvis and ureter (kidney (renal cell) cancer); carcinoma of unknown primary; childhood cancer of unknown primary; unusual cancers of childhood; ureter and renal pelvis, transitional cell cancer (kidney (renal cell) cancer; urethral cancer; uterine cancer, endometrial; uterine sarcoma; vaginal cancer; childhood vaginal cancer; vascular tumors (soft tissue sarcoma); vulvar cancer; wilms tumor and other childhood kidney tumors; or cancer in young adults.

The methods, compositions, and systems of the present disclosure may be used to detect a cardiovascular disease state. As used herein, the terms “cardiovascular disease” (CVD) or “cardiovascular disorder” are used to classify numerous conditions affecting the heart, heart valves, and vasculature (e.g., veins and arteries) of the body and encompasses diseases and conditions including, but not limited to atherosclerosis, myocardial infarction, acute coronary syndrome, angina, congestive heart failure, aortic aneurysm, aortic dissection, iliac or femoral aneurysm, pulmonary embolism, atrial fibrillation, stroke, transient ischemic attack, systolic dysfunction, diastolic dysfunction, myocarditis, atrial tachycardia, ventricular fibrillation, endocarditis, peripheral vascular disease, and coronary artery disease (CAD). Further, the term cardiovascular disease refers to conditions in subjects that ultimately have a cardiovascular event or cardiovascular complication, referring to the manifestation of an adverse condition in a subject brought on by cardiovascular disease, such as sudden cardiac death or acute coronary syndrome, including, but not limited to, myocardial infarction, unstable angina, aneurysm, stroke, heart failure, non-fatal myocardial infarction, stroke, angina pectoris, transient ischemic attacks, aortic aneurysm, aortic dissection, cardiomyopathy, abnormal cardiac catheterization, abnormal cardiac imaging, stent or graft revascularization, risk of experiencing an abnormal stress test, risk of experiencing abnormal myocardial perfusion, and death.

As used herein, the ability to detect, diagnose or prognose cardiovascular disease, for example, atherosclerosis, can include determining if the patient is in a pre-stage of cardiovascular disease, has developed early, moderate or severe forms of cardiovascular disease, or has suffered one or more cardiovascular event or complication associated with cardiovascular disease.

Atherosclerosis (also known as arteriosclerotic vascular disease or ASVD) is a cardiovascular disease in which an artery-wall thickens as a result of invasion and accumulation and deposition of arterial plaques containing white blood cells on the innermost layer of the walls of arteries resulting in the narrowing and hardening of the arteries. The arterial plaque is an accumulation of macrophage cells or debris, and contains lipids (cholesterol and fatty acids), calcium and a variable amount of fibrous connective tissue. Diseases associated with atherosclerosis include, but are not limited to, atherothrombosis, coronary heart disease, deep venous thrombosis, carotid artery disease, angina pectoris, peripheral arterial disease, chronic kidney disease, acute coronary syndrome, vascular stenosis, myocardial infarction, aneurysm or stroke. In one embodiment the automated apparatuses, compositions, and methods of the present disclosure may distinguish the different stages of atherosclerosis, including, but not limited to, the different degrees of stenosis in a subject.

In some cases, the disease or disorder detected by the methods, compositions, or systems of the present disclosure is an endocrine disease. The term “endocrine disease” is used to refer to a disorder associated with dysregulation of endocrine system of a subject. Endocrine diseases may result from a gland producing too much or too little of an endocrine hormone causing a hormonal imbalance, or due to the development of lesions (such as nodules or tumors) in the endocrine system, which may or may not affect hormone levels. Suitable endocrine diseases able to be treated include, but are not limited to, e.g., Acromegaly, Addison’s Disease, Adrenal Cancer, Adrenal Disorders, Anaplastic Thyroid Cancer, Cushing’s Syndrome, De Quervain’s Thyroiditis, Diabetes, Follicular Thyroid Cancer, Gestational Diabetes, Goiters, Graves’ Disease, Growth Disorders, Growth Hormone Deficiency, Hashimoto’s Thyroiditis, Hurthle Cell Thyroid Cancer, Hyperglycemia, Hyperparathyroidism, Hyperthyroidism, Hypoglycemia, Hypoparathyroidism, Hypothyroidism, Low Testosterone, Medullary Thyroid Cancer, MEN 1, MEN 2A, MEN 2B, Menopause, Metabolic Syndrome, Obesity, Osteoporosis, Papillary Thyroid Cancer, Parathyroid Diseases, Pheochromocytoma, Pituitary Disorders, Pituitary Tumors, Polycystic Ovary Syndrome, Prediabetes, Silent, Thyroiditis, Thyroid Cancer, Thyroid Diseases, Thyroid Nodules, Thyroiditis, Turner Syndrome, Type 1 Diabetes, Type 2 Diabetes, and the like.

In some cases, the disease or disorder detected by methods, compositions, or systems of the present disclosure is an inflammatory disease. As referred to herein, inflammatory disease refers to a disease caused by uncontrolled inflammation in the body of a subject. Inflammation is a biological response of the subject to a harmful stimulus which may be external or internal such as pathogens, necrosed cells and tissues, irritants etc. However, when the inflammatory response becomes abnormal, it results in self-tissue injury and may lead to various diseases and disorders. Inflammatory diseases can include, but are not limited to, asthma, glomerulonephritis, inflammatory bowel disease, rheumatoid arthritis, hypersensitivities, pelvic inflammatory disease, autoimmune diseases, arthritis; necrotizing enterocolitis (NEC), gastroenteritis, pelvic inflammatory disease (PID), emphysema, pleurisy, pyelitis, pharyngitis, angina, acne vulgaris, urinary tract infection, appendicitis, bursitis, colitis, cystitis, dermatitis, phlebitis, rhinitis, tendonitis, tonsillitis, vasculitis, autoimmune diseases; celiac disease; chronic prostatitis, hypersensitivities, reperfusion injury; sarcoidosis, transplant rejection, vasculitis, interstitial cystitis, hay fever, periodontitis, atherosclerosis, psoriasis, ankylosing spondylitis, juvenile idiopathic arthritis, Behcet’s disease, spondyloarthritis, uveitis, systemic lupus erythematosus, and cancer. For example, the arthritis includes rheumatoid arthritis, psoriatic arthritis, osteoarthritis or juvenile idiopathic arthritis, and the like.

The methods, compositions, and systems of the present disclosure may detect a neurological disease state. Neurological disorders or neurological diseases are used interchangeably and refer to diseases of the brain, spine and the nerves that connect them. Neurological diseases include, but are not limited to, brain tumors, epilepsy, Parkinson’s disease, Alzheimer’s disease, ALS, arteriovenous malformation, cerebrovascular disease, brain aneurysms, epilepsy, multiple sclerosis, Peripheral Neuropathy, Post-Herpetic Neuralgia, stroke, frontotemporal dementia, demyelinating disease (including but are not limited to, multiple sclerosis, Devic’s disease (i.e. neuromyelitis optica), central pontine myelinolysis, progressive multifocal leukoencephalopathy, leukodystrophies, Guillain-Barre syndrome, progressing inflammatory neuropathy, Charcot-Marie-Tooth disease, chronic inflammatory demyelinating polyneuropathy, and anti-MAG peripheral neuropathy) and the like. Neurological disorders also include immune-mediated neurological disorders (IMNDs), which include diseases with at least one component of the immune system reacts against host proteins present in the central or peripheral nervous system and contributes to disease pathology. IMNDs may include, but are not limited to, demyelinating disease, paraneoplastic neurological syndromes, immune-mediated encephalomyelitis, immune-mediated autonomic neuropathy, myasthenia gravis, autoantibody-associated encephalopathy, and acute disseminated encephalomyelitis.

Methods, systems, and/or apparatuses of the present disclosure may be able to accurately distinguish between patients with or without Alzheimer’s disease. These may also be able to detect patients who are pre-symptomatic and may develop Alzheimer’s disease several years after the screening. This provides advantages of being able to treat a disease at a very early stage, even before development of the disease.

The methods, compositions, and systems of the present disclosure can detect a pre-disease stage of a disease or disorder. A pre-disease stage is a stage at which the patient has not developed any signs or symptoms of the disease. A pre-cancerous stage would be a stage in which cancer or tumor or cancerous cells have not be identified within the subject. A pre-neurological disease stage would be a stage in which a person has not developed one or more symptom of the neurological disease. The ability to diagnose a disease before one or more sign or symptom of the disease is present allows for close monitoring of the subject and the ability to treat the disease at a very early stage, increasing the prospect of being able to halt progression or reduce the severity of the disease.

The methods, compositions, and systems of the present disclosure may detect the early stages of a disease or disorder. Early stages of the disease can refer to when the first signs or symptoms of a disease may manifest within a subject. The early stage of a disease may be a stage at which there are no outward signs or symptoms. For example, in Alzheimer’s disease an early stage may be a pre-Alzheimer’s stage in which no symptoms are detected yet the patient will develop Alzheimer’s months or years later.

Identifying a disease in either pre-disease development or in the early states may often lead to a higher likelihood for a positive outcome for the patient. For example, diagnosing cancer at an early stage (stage 0 or stage 1) can increase the likelihood of survival by over 80%. Stage 0 cancer can describe a cancer before it has begun to spread to nearby tissues. This stage of cancer is often highly curable, usually by removing the entire tumor with surgery. Stage 1 cancer may usually be a small cancer or tumor that has not grown deeply into nearby tissue and has not spread to lymph nodes or other parts of the body.

In some cases, the methods, compositions, and systems of the present disclosure are able to detect intermediate stages of the disease. Intermediate states of the disease describe stages of the disease that have passed the first signs and symptoms and the patient is experiencing one or more symptom of the disease. For example, for cancer, stage II or III cancers are considered intermediate stages, indicating larger cancers or tumors that have grown more deeply into nearby tissue. In some instances, stage II or III cancers may have also spread to lymph nodes but not to other parts of the body.

Further, the methods, compositions, and systems of the present disclosure may be able to detect late or advanced stages of the disease. Late or advanced stages of the disease may also be called “severe” or “advanced” and usually indicates that the subject is suffering from multiple symptoms and effects of the disease. For example, severe stage cancer includes stage IV, where the cancer has spread to other organs or parts of the body and is sometimes referred to as advanced or metastatic cancer.

The methods of the present disclosure can include processing the biomolecule corona data of a sample against a collection of biomolecule corona datasets representative of a plurality of diseases and/or a plurality of disease states to determine if the sample indicates a disease and/or disease state. For example, samples can be collected from a population of subjects over time. Once the subjects develop a disease or disorder, the present disclosure allows for the ability to characterize and detect the changes in biomolecule fingerprints over time in the subject by computationally analyzing the biomolecule fingerprint of the sample from the same subject before they have developed a disease to the biomolecule fingerprint of the subject after they have developed the disease. Samples can also be taken from cohorts of patients who all develop the same disease, allowing for analysis and characterization of the biomolecule fingerprints that are associated with the different stages of the disease for these patients (e.g. from pre-disease to disease states).

In some cases, the methods, compositions, and systems of the present disclosure are able to distinguish not only between different types of diseases, but also between the different stages of the disease (e.g. early stages of cancer). This can comprise distinguishing healthy subjects from pre-disease state subjects. The pre-disease state may be stage 0 or stage 1 cancer, a neurodegenerative disease, dementia, a coronary disease, a kidney disease, a cardiovascular disease (e.g., coronary artery disease), diabetes, or a liver disease. Distinguishing between different stages of the disease can comprise distinguishing between two stages of a cancer (e.g., stage 0 vs stage 1 or stage 1 vs stage 3).

A method of the present disclosure may identify biomarkers associated with a biological state. For example, a biomolecule corona assay may identify cancer-specific mutant proteins with mass spectrometry, and correlate the presence of the mutant proteins to a form of cancer. A method of the present disclosure may also identify a biological state based on the patterns of biomolecules present in a sample, enriched from a sample, or disposed within a biomolecule corona. For example, a method of the present disclosure may identify a biological state based upon the presence or relative abundances of 10 non-biomarker proteins in two biomolecule coronas of two separate particles, or based on the abundance ratios of albumin, globulins, and a specific cytokine in a biomolecule corona. Biomolecule identification may be performed with probes comprising known binding specificities. A probe may comprise specificity for a single target biomolecule, such as a specific protein. A probe may comprise specificity for a particular form of a target biomolecule, such as a particular conformation or post-translationally modified state of a protein. For example, a first probe may comprise specificity for hemoglobin A (HbA), while a second probe may comprise specificity only for N-terminal glycated hemoglobin A (HbA1c). A probe may comprise specificity for a set of biomolecules. For example, a probe may comprise binding specificity for all RAS proteins (e.g., KRAS, HRAS, NRAS, some mutant forms thereof, and phosphorylated versions thereof). A library of probes with known binding specificities may be referred to as an ‘a priori’ probe library herein.

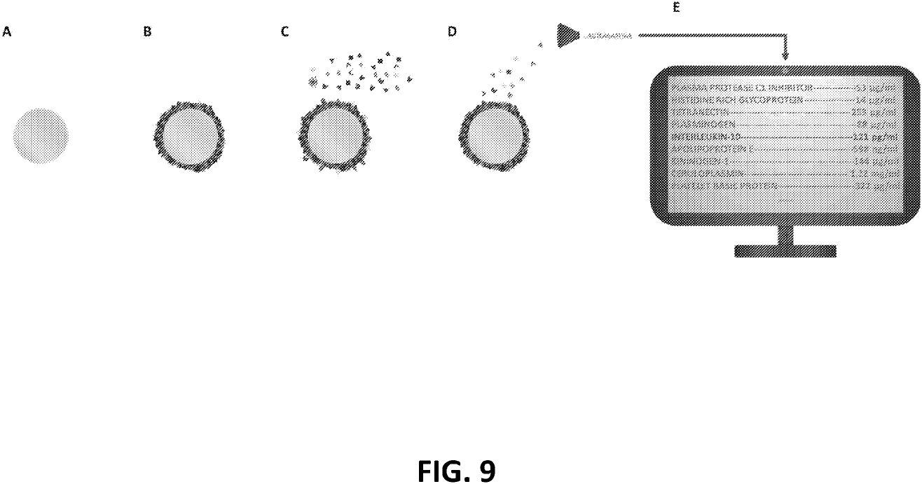

FIG. 9 illustrates a method for determining the biological state of a patient by contacting a sample from the patient with an a priori probe library. The probes used in this assay have been evolved to bind specific biomolecular targets, so that the pattern of probe binding can be used to quantify the concentrations of specific biomolecules from a sample. Each type of probe contains a unique identifier barcode, allowing each probe to be identified and quantified by NGS.

FIG. 9 Panel A shows a bare particle prior to contacting a sample. FIG. 9 Panel B shows the particle after it has contacted the sample from the patient, resulting in the formation of a biomolecule corona. FIG. 9 Panel C shows the particle being contacted by the a priori probe library. Probes that do not bind to the biomolecule corona are removed from the sample through multiple wash cycles. FIG. 9 Panel D shows the biomolecule corona bound probes being desorbed from the particle and identified by NGS. Panel E shows the results of the assay, wherein the probe binding pattern has been used to determine the concentrations of multiple proteins in the patient’s sample. Such a pattern may be used to identify a biological state of sample.

Alternatively or in combination with biomolecule identification, a method of the present disclosure may identify a biological state based on unannotated data. In such cases, data features may be analyzed without identification of the biomolecules to which they correspond. The present disclosure provides a range of probes with degrees of binding non-specificity, such that the probe may bind to a range of biomolecules present in a sample. In some cases, methods utilizing such probes may not identify a specific biomolecule present in a sample. However, the pattern of such “naive” probes which bind to a sample or a subset of a sample (e.g., a biomolecule corona) may identify a particular biological state, such as cancer. Such a method may also involve direct analysis of mass spectrometric data, optical data, electrochemical data, or other data collected on the biomolecules of the sample.

An example of such a method is provided in FIG. 8, which outlines a protocol for determining the biological state of a patient by contacting a sample from the patient with a naive library of probes. The top and bottom rows illustrate parallel assays on biological samples from different patients. FIG. 8 Panel A shows bare particles prior to contacting samples. FIG. 8 Panel B shows the particles following contact with the samples and formation of biomolecule coronas. FIG. 8 Panel C shows each particle being contacted with a library of probes. The specific targets for this library of probes are unknown. Instead, a computational model has been trained to use the pattern of probe binding to identify the disease state of a subject. As is shown in FIG. 8 Panel C, probes which target biomolecules present on the surface of a biomolecule corona may bind to the biomolecule corona. Thus, a probe binding pattern to a biomolecule corona is partially determined by the composition of the biomolecule corona. Unbound probes can be removed through multiple series of washes.

In FIG. 8 Panel D, the remaining probes are eluted from the surfaces of the biomolecule coronas and detected. Each type of probe has a unique absorbance profile, allowing the corona-bound probes to be quickly identified and quantified by absorbance within a diode array. FIG. 8 Panel E shows the results of the assays. Based upon the patterns of probe binding to each biomolecule corona, the computational algorithm is able to identify the first patient as healthy, and the second patient as diabetic.

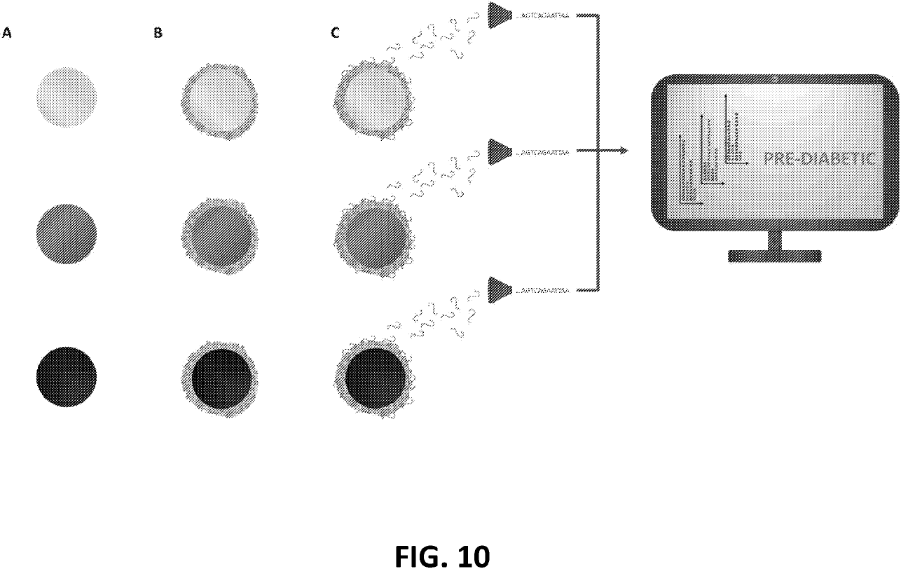

The information obtained from a probe binding assay may include the portions of a sample to which a probe binds, as well as the probe abundances therefrom. A plurality of probes may be contacted to a plurality of biomolecule coronas derived from the same subject, patient, or sample. FIG. 10 illustrates a method for determining the biological state of a patient using a particle array and a probe library. In this specific illustration, the probe library is comprised of DNA aptamers that are identifiable by NGS and that are each capable of binding multiple targets. The probe library may have been subjected to multiple rounds of evolution to differently bind to plasma samples from subjects with different biological states. For example, the probe library may have been evolved to distinguish between diabetic, pre-diabetic, and non-diabetic patients. The identities of the probes and their targets may be known, partially known, or unknown. FIG. 10 Panel A shows an array of three particles. The three particles differ in composition and surface properties. FIG. 10 Panel B shows the particle array following contact with a sample from the patient. The differences in surface properties of the three particles lead to the formation of different biomolecule coronas on the particles. FIG. 10 Panel C shows each particle in the particle array being separately contacted with a probe library. The probes that do not bind to the biomolecule coronas are detected on an individual particle basis by NGS. The pattern of probe non-binding is used to fingerprint each sample, and to determine whether the patient that provided the sample is diabetic or pre-diabetic.

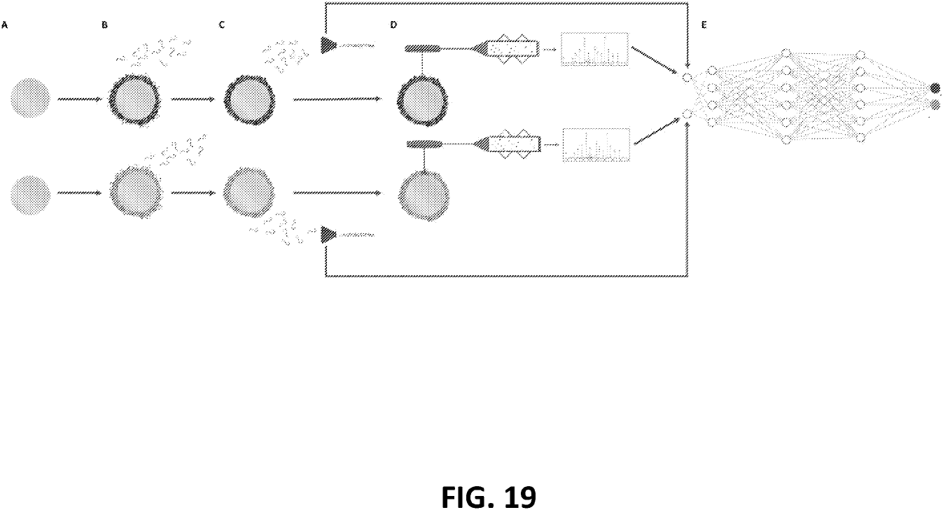

A classifier may be trained to distinguish between sample types based on probe binding, direct biomolecule analysis (e.g., mass spectrometric analysis), or a combination thereof. FIG. 19 outlines a method for training a classifier to distinguish between multiple samples (e.g., different biological states of a sample type) based on differential probe binding. In this method, one particle is contacted with a sample from a healthy patient (top row), and a second particle is contacted with a sample from a patient carrying the disease (bottom row), leading to different biomolecule coronas on the two particles. FIG. 19 Panel A shows a set of particles prior to contact with biological samples. Each particle is then contacted with a biological sample, resulting in the formation of a biomolecule corona. As is shown in FIG. 19 Panel B, each particle is then contacted with a library of probes. A subset of the probes bind to each corona, while the remainder of the probes are washed away. As shown in FIG. 19 Panel C, the corona-bound probes are desorbed and sequenced. FIG. 19 Panel D shows an optional step of mass spectrometric biomolecule corona analysis, which may generate further data for classifier training. Next, as shown in FIG. 19 Panel E, the sequencing data (and optionally the mass spectrometry data) may be used to train a computational algorithm (e.g., a neural network) to distinguish the disease state from the healthy state. This training method may not require any knowledge of the targets or binding affinities of the probes, but rather may utilize probe binding patterns to distinguish the biological states associated with the input samples. The trained classifier may then be applied to probe binding and optionally mass spectrometric data on unknown samples to identify a biological state of the unknown sample.

Various aspects of the present disclosure provide non-particle-based methods for combined probe and sensor element analysis. A sensor element may be a material or species which collects molecules from a sample (e.g., which collects biomolecules from a biological sample). While many methods of the present disclosure utilize particles to species within biomolecule coronas, a method may utilize alternative forms of sensor elements, such as filters, polymer matrices, surfaces, rods (e.g., nanorods or nanotubes), quantum dots, resins, or combinations thereof.

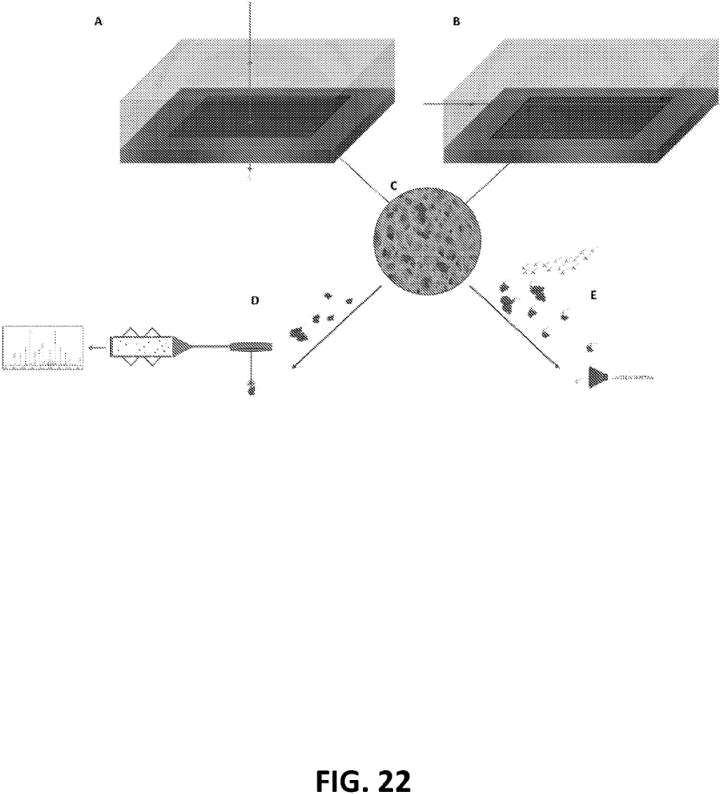

FIG. 22 illustrates a method for assaying a sample with a non-particle sensor element and a probe library. In this example, the sensor element comprises a semipermeable matrix configured to collect biomolecules from a sample flowing through (FIG. 22 Panel A) or past (FIG. 22 Panel B). The biomolecule affinity of the semipermeable matrix may be dependent on its chemical and physical properties (e.g., charge, hydrophobicity, surface functionalization), as well as the sizes of its pores. Thus, two different semipermeable matrices may collect different subsets of biomolecules upon contacting the same sample.

Biomolecules may adsorb on or within the semipermeable matrix (FIG. 22 Panel C). Collected biomolecules can be eluted from the semipermeable matrix and subjected to further enrichment, treatment, and analysis. For example, a biomolecule collected on a semipermeable matrix may be eluted and analyzed by mass spectrometry (FIG. 22 Panel D), assayed with a library of affinity reagents (FIG. 22 Panel E), contacted to a particle, or any combination thereof.

### Proteograph

Any of the affinity reagents, probes, and libraries thereof (e.g., DNA encoded libraries of probes) can be used in conjunction with Proteograph analysis. Proteograph analysis may combine a multi-particle type protein corona strategy with mass spectrometry (MS). Advantageously, by combining the first part of the Protograph workflow (sample incubation with particles and separation of particles) with the affinity reagents disclosed herein can eliminate the need for MS identification of proteins. Particle types included in the particle panels disclosed herein can be superparamagnetic and are, thus, rapidly separated or isolated from unbound protein (proteins that have not adsorbed onto the surface of a particle to form the corona) in a sample, after incubation of the particle in the sample.

A particle of the present disclosure may be contacted with a biological sample (e.g., a biofluid) to form a biomolecule corona. The particle and biomolecule corona may be separated from the biological sample, for example by centrifugation, magnetic separation, filtration, or gravitational separation. The particle types and biomolecule corona may be separated from the biological sample using a number of separation techniques. Non-limiting examples of separation techniques include comprises magnetic separation, column-based separation, filtration, spin column-based separation, centrifugation, ultracentrifugation, density or gradient-based centrifugation, gravitational separation, or any combination thereof. A protein corona analysis may be performed on the separated particle and biomolecule corona. A protein corona analysis may comprise identifying one or more proteins in the biomolecule corona, for example by mass spectrometry. In some embodiments, a single particle type may be contacted to a biological sample. In some embodiments, a plurality of particle types may be contacted to a biological sample. The plurality of particle types may be combined and contacted to the biological sample in a single sample volume. The plurality of particle types may be sequentially contacted to a biological sample and separated from the biological sample prior to contacting a subsequent particle type to the biological sample. Protein corona analysis of the biomolecule corona may compress the dynamic range of the analysis compared to a total protein analysis method.

### Computer Control Systems

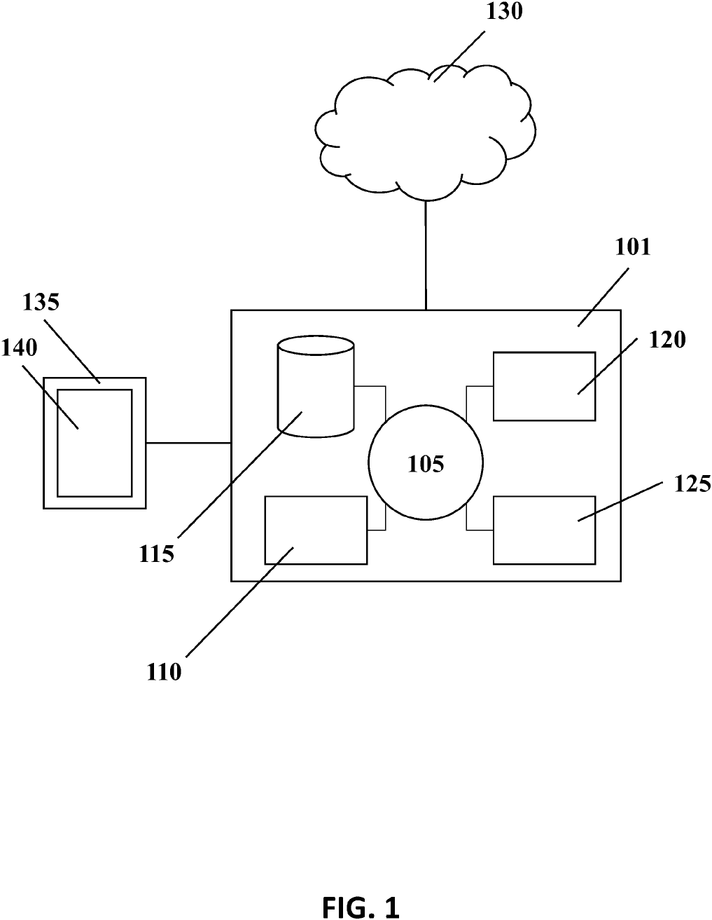

The present disclosure provides computer control systems that are programmed to implement methods of the disclosure. FIG. 1 shows a computer system that is programmed or otherwise configured to implement methods provided herein. The computer system 101 can regulate various aspects of the assays disclosed herein, which are capable of being automated (e.g., movement of any of the reagents disclosed herein on a substrate). The computer system 101 can be an electronic device of a user or a computer system that is remotely located with respect to the electronic device. The electronic device can be a mobile electronic device.

The computer system 101 includes a central processing unit (CPU, also “processor” and “computer processor” herein) 105, which can be a single core or multi core processor, or a plurality of processors for parallel processing. The computer system 101 also includes memory or memory location 110 (e.g., random-access memory, read-only memory, flash memory), electronic storage unit 115 (e.g., hard disk), communication interface 120 (e.g., network adapter) for communicating with one or more other systems, and peripheral devices 125, such as cache, other memory, data storage and/or electronic display adapters. The memory 110, storage unit 115, interface 120 and peripheral devices 125 are in communication with the CPU 105 through a communication bus (solid lines), such as a motherboard. The storage unit 115 can be a data storage unit (or data repository) for storing data. The computer system 101 can be operatively coupled to a computer network (“network”) 130 with the aid of the communication interface 120. The network 130 can be the Internet, an internet and/or extranet, or an intranet and/or extranet that is in communication with the Internet. The network 130 in some cases is a telecommunication and/or data network. The network 130 can include one or more computer servers, which can enable distributed computing, such as cloud computing. The network 130, in some cases with the aid of the computer system 101, can implement a peer-to-peer network, which may enable devices coupled to the computer system 101 to behave as a client or a server.

The CPU 105 can execute a sequence of machine-readable instructions, which can be embodied in a program or software. The instructions may be stored in a memory location, such as the memory 110. The instructions can be directed to the CPU 105, which can subsequently program or otherwise configure the CPU 105 to implement methods of the present disclosure. Examples of operations performed by the CPU 105 can include fetch, decode, execute, and writeback.

The CPU 105 can be part of a circuit, such as an integrated circuit. One or more other components of the system 101 can be included in the circuit. In some cases, the circuit is an application specific integrated circuit (ASIC).

The storage unit 115 can store files, such as drivers, libraries and saved programs. The storage unit 115 can store user data, e.g., user preferences and user programs. The computer system 101 in some cases can include one or more additional data storage units that are external to the computer system 101, such as located on a remote server that is in communication with the computer system 101 through an intranet or the Internet.

The computer system 101 can communicate with one or more remote computer systems through the network 130. For instance, the computer system 101 can communicate with a remote computer system of a user. Examples of remote computer systems include personal computers (e.g., portable PC), slate or tablet PC’s (e.g., Apple® iPad, Samsung® Galaxy Tab), telephones, Smart phones (e.g., Apple® iPhone, Android-enabled device, Blackberry®), or personal digital assistants. The user can access the computer system 101 via the network 130.

Methods as described herein can be implemented by way of machine (e.g., computer processor) executable code stored on an electronic storage location of the computer system 101, such as, for example, on the memory 110 or electronic storage unit 115. The machine executable or machine readable code can be provided in the form of software. During use, the code can be executed by the processor 105. In some cases, the code can be retrieved from the storage unit 115 and stored on the memory 110 for ready access by the processor 105. In some situations, the electronic storage unit 115 can be precluded, and machine-executable instructions are stored on memory 110.

The code can be pre-compiled and configured for use with a machine having a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 101, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 101 can include or be in communication with an electronic display 135 that comprises a user interface (UI) 140 for providing, for example a readout of the proteins identified using the methods disclosed herein. Examples of UI’s include, without limitation, a graphical user interface (GUI) and web-based user interface.

Methods and systems of the present disclosure can be implemented by way of one or more algorithms. An algorithm can be implemented by way of software upon execution by the central processing unit 105.

Determination, analysis or statistical classification is done by methods known in the art, including, but not limited to, for example, a wide variety of supervised and unsupervised data analysis and clustering approaches such as hierarchical cluster analysis (HCA), principal component analysis (PCA), Partial least squares Discriminant Analysis (PLSDA), machine learning (also known as random forest), logistic regression, decision trees, support vector machine (SVM), k-nearest neighbors, naive bayes, linear regression, polynomial regression, SVM for regression, K-means clustering, and hidden Markov models, among others. The computer system can perform various aspects of analyzing the protein sets or protein corona of the present disclosure, such as, for example, comparing/analyzing the biomolecule corona of several samples to determine with statistical significance what patterns are common between the individual biomolecule coronas to determine a protein set that is associated with the biological state. The computer system can be used to develop classifiers to detect and discriminate different protein sets or protein corona (e.g., characteristic of the composition of a protein corona). Data collected from the presently disclosed sensor array can be used to train a machine learning algorithm, specifically an algorithm that receives array measurements from a patient and outputs specific biomolecule corona compositions from each patient. Before training the algorithm, raw data from the array can be first denoised to reduce variability in individual variables.

Machine learning can be generalized as the ability of a learning machine to perform accurately on new, unseen examples/tasks after having experienced a learning data set. Machine learning may include the following concepts and methods. Supervised learning concepts may include AODE; Artificial neural network, such as Backpropagation, Autoencoders, Hopfield networks, Boltzmann machines, Restricted Boltzmann Machines, and Spiking neural networks; Bayesian statistics, such as Bayesian network and Bayesian knowledge base; Case-based reasoning; Gaussian process regression; Gene expression programming; Group method of data handling (GMDH); Inductive logic programming; Instance-based learning; Lazy learning; Learning Automata; Learning Vector Quantization; Logistic Model Tree; Minimum message length (decision trees, decision graphs, etc.), such as Nearest Neighbor Algorithm and Analogical modeling; Probably approximately correct learning (PAC) learning; Ripple down rules, a knowledge acquisition methodology; Symbolic machine learning algorithms; Support vector machines; Random Forests; Ensembles of classifiers, such as Bootstrap aggregating (bagging) and Boosting (meta-algorithm); Ordinal classification; Information fuzzy networks (IFN); Conditional Random Field; ANOVA; Linear classifiers, such as Fisher’s linear discriminant, Linear regression, Logistic regression, Multinomial logistic regression, Naive Bayes classifier, Perceptron, Support vector machines; Quadratic classifiers; k-nearest neighbor; Boosting; Decision trees, such as C4.5, Random forests, ID3, CART, SLIQ SPRINT; Bayesian networks, such as Naive Bayes; and Hidden Markov models. Unsupervised learning concepts may include; Expectation-maximization algorithm; Vector Quantization; Generative topographic map; Information bottleneck method; Artificial neural network, such as Self-organizing map; Association rule learning, such as, Apriori algorithm, Eclat algorithm, and FPgrowth algorithm; Hierarchical clustering, such as Singlelinkage clustering and Conceptual clustering; Cluster analysis, such as, K-means algorithm, Fuzzy clustering, DBSCAN, and OPTICS algorithm; and Outlier Detection, such as Local Outlier Factor. Semi-supervised learning concepts may include; Generative models; Low-density separation; Graph-based methods; and Co-training. Reinforcement learning concepts may include; Temporal difference learning; Q-learning; Learning Automata; and SARSA. Deep learning concepts may include; Deep belief networks; Deep Boltzmann machines; Deep Convolutional neural networks; Deep Recurrent neural networks; and Hierarchical temporal memory. A computer system may be adapted to implement a method described herein. The system includes a central computer server that is programmed to implement the methods described herein. The server includes a central processing unit (CPU, also “processor”) which can be a single core processor, a multi core processor, or plurality of processors for parallel processing. The server also includes memory (e.g., random access memory, read-only memory, flash memory); electronic storage unit (e.g. hard disk); communications interface (e.g., network adaptor) for communicating with one or more other systems; and peripheral devices which may include cache, other memory, data storage, and/or electronic display adaptors. The memory, storage unit, interface, and peripheral devices are in communication with the processor through a communications bus (solid lines), such as a motherboard. The storage unit can be a data storage unit for storing data. The server is operatively coupled to a computer network (“network”) with the aid of the communications interface. The network can be the Internet, an intranet and/or an extranet, an intranet and/or extranet that is in communication with the Internet, a telecommunication or data network. The network in some cases, with the aid of the server, can implement a peer-to-peer network, which may enable devices coupled to the server to behave as a client or a server.

The storage unit can store files, such as subject reports, and/or communications with the data about individuals, or any aspect of data associated with the present disclosure.

The computer server can communicate with one or more remote computer systems through the network. The one or more remote computer systems may be, for example, personal computers, laptops, tablets, telephones, Smart phones, or personal digital assistants.

In some applications the computer system includes a single server. In other situations, the system includes multiple servers in communication with one another through an intranet, extranet and/or the internet.

The server can be adapted to store measurement data or a database as provided herein, patient information from the subject, such as, for example, medical history, family history, demographic data and/or other clinical or personal information of potential relevance to a particular application. Such information can be stored on the storage unit or the server and such data can be transmitted through a network.

Methods as described herein can be implemented by way of machine (or computer processor) executable code (or software) stored on an electronic storage location of the server, such as, for example, on the memory, or electronic storage unit. During use, the code can be executed by the processor. In some cases, the code can be retrieved from the storage unit and stored on the memory for ready access by the processor. In some situations, the electronic storage unit can be precluded, and machine-executable instructions are stored on memory. Alternatively, the code can be executed on a second computer system.

Aspects of the systems and methods provided herein, such as the server, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical, and electromagnetic waves, such as used across physical interfaces between local devices, through wired and optical landline networks and over various air-links. The physical elements that carry such waves, such as wired or wireless likes, optical links, or the like, also may be considered as media bearing the software. As used herein, unless restricted to non-transitory, tangible “storage” media, terms such as computer or machine “readable medium” can refer to any medium that participates in providing instructions to a processor for execution.

The computer systems described herein may comprise computer-executable code for performing any of the algorithms or algorithms-based methods described herein. In some applications the algorithms described herein will make use of a memory unit that is comprised of at least one database.

Data relating to the present disclosure can be transmitted over a network or connections for reception and/or review by a receiver. The receiver can be but is not limited to the subject to whom the report pertains; or to a caregiver thereof, e.g., a health care provider, manager, other health care professional, or other caretaker; a person or entity that performed and/or ordered the analysis. The receiver can also be a local or remote system for storing such reports (e.g. servers or other systems of a “cloud computing” architecture). In one embodiment, a computer-readable medium includes a medium suitable for transmission of a result of an analysis of a biological sample using the methods described herein.

Aspects of the systems and methods provided herein can be embodied in programming. Various aspects of the technology may be thought of as “products” or “articles of manufacture” typically in the form of machine (or processor) executable code and/or associated data that is carried on or embodied in a type of machine readable medium. Machineexecutable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide nontransitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer- executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

### Classification of Protein Coronas and Affinity Reagent Binding Using Machine Learning

The method of determining a set of proteins associated with the disease or disorder and/or disease state include the analysis of the corona of at least two samples. This determination, analysis or statistical classification may be done by methods known in the art, including, but not limited to, for example, a wide variety of supervised and unsupervised data analysis, machine learning, deep learning, and clustering approaches including hierarchical cluster analysis (HCA), principal component analysis (PCA), Partial least squares Discriminant Analysis (PLS-DA), random forest, logistic regression, decision trees, support vector machine (SVM), k-nearest neighbors, naive Bayes, linear regression, polynomial regression, SVM for regression, K-means clustering, and hidden Markov models, among others. In other words, the proteins in the corona of each sample are compared/analyzed with each other to determine with statistical significance what patterns are common between the individual corona to determine a set of proteins that is associated with the disease or disorder or disease state.

Generally, machine learning algorithms are used to construct models that accurately assign class labels to examples based on the input features that describe the example. In some case it may be advantageous to employ machine learning and/or deep learning approaches for the methods described herein. For example, machine learning can be used to associate the protein corona with various disease states (e.g. no disease, precursor to a disease, having early or late stage of the disease, etc.). For example, in some cases, one or more machine learning algorithms are employed in connection with a method of the invention to analyze data detected and obtained by the protein corona and sets of proteins derived therefrom. For example, in one embodiment, machine learning can be coupled with the sensor array described herein to determine not only if a subject has a pre-stage of cancer, cancer or does not have or develop cancer, but also to distinguish the type of cancer.

### NUMBERED EMBODIMENTS

Some aspects include any of the following numbered embodiments.

1. A method of assaying a biomolecule in a sample, the method comprising:
a) incubating a particle in the sample, thereby adsorbing biomolecules from the sample onto the particle to form a biomolecule corona;
b) incubating the particle with a probe comprising (i) an affinity reagent and (ii) a barcode, wherein the affinity reagent binds to a biomolecule of the biomolecule corona; and
c) assaying for the presence, absence or amount of the probe, thereby assaying for the presence, absence or amount of the biomolecule of the biomolecule corona.

2. The method of embodiment 1, wherein the affinity reagent comprises an antibody, a peptide, a nucleic acid ligand, a Fab, a Fab2, an scFv, an scFab, an aptamer, a polypeptide ligand scaffold, a ligand, or a chemical moiety.

3. The method of embodiment 2, wherein the peptide comprises an adnectin, abamer, affibody, or nanobody.

4. The method of any one of embodiments 1-3, wherein the affinity reagent is from about 1 nm to about 35 nm in a dimension.

5. The method of any one of embodiments 1-4, wherein the affinity reagent comprises a molecular mass from 200 Da to 200 kDa.

6. The method of any one of embodiments 1-5, wherein the barcode comprises a single stranded nucleic acid, a double stranded nucleic acid, or a sticky end of a nucleic acid.

7. The method of any one of embodiments 1-6, wherein the probe is present in a plurality of probes.

8. The method of embodiment 7, wherein the plurality of probes comprise different affinity reagents.

9. The method of embodiment 7 or 8, wherein the plurality of probes comprise a library of barcodes.

10. The method of any one of embodiments 7-9, wherein each probe of the plurality of probes comprises a unique barcode.

11. The method of embodiment 9 or 10, wherein the library of barcodes comprises from 50 to 10¹⁰ distinct barcodes.

12. The method of any one of embodiments 9-11, wherein the library of barcodes comprises a combinatorially generated nucleic acid library.

13. The method of any one of embodiments 9-12, wherein the library of barcodes comprises double stranded DNA barcodes.

14. The method of any one of embodiments 9-13, wherein the barcodes comprise barcode nucleotide sequences.

15. The method of embodiment 14, wherein affinity reagents of the plurality of probes bind different biomolecules, and wherein different biomolecules may be identified by the barcode nucleotide sequences of probes that bind to the different biomolecules.

16. The method of embodiment 15, wherein probes comprising affinity reagents that bind a biomolecule include a first barcode nucleotide sequence, and probes comprising affinity reagents that bind another biomolecule include a second barcode nucleotide sequence.

17. The method of any one of embodiments 7-16, wherein a first probe of the plurality of probes comprises a first affinity reagent that binds a first biomolecule, and a second probe of the plurality comprises a second affinity reagent that binds a different region of the first biomolecule.

18. The method of any one of embodiments 7-16, wherein a first probe of the plurality of probes comprises a first affinity reagent that binds a first biomolecule, and a second probe of the plurality of probes comprises a second affinity reagent that binds a second biomolecule in close proximity with the first biomolecule.

19. The method of embodiment 17 or 18, wherein a barcode of the first probe hybridizes with a barcode of the second probe.

20. The method of embodiment 19, further comprising extending the 3′ ends of the hybridized barcodes of the first and second probes.

21. The method of embodiment 19, wherein the barcodes of the first and second probes comprise sticky ends that hybridize together, and further comprising ligating the sticky ends.

22. The method of any one of embodiments 14-21, wherein the assaying of c) comprises sequencing the barcode nucleotide sequences.

23. The method of any one of embodiments 14-22, wherein the barcode nucleotide sequences comprise primer sequences.

24. The method of any one of embodiments 14-23, wherein the assaying of c) comprises amplification.

25. The method of embodiment 24, wherein the barcode nucleotide sequences or a segment of the barcode nucleotide sequences is amplified prior to sequencing.

26. The method of embodiment 24 or 25, wherein the amplification comprises thermal cycling amplification.

27. The method of embodiment 26, wherein the thermal cycling amplification comprises polymerase chain reaction.

28. The method of embodiment 24 or 25, wherein the amplification comprises isothermal amplification.

29. The method of any one of embodiments 22-28, wherein the sequencing comprises next generation sequencing.

30. The method of any one of embodiments 22-29, wherein the sequencing is nanopore sequencing.

31. The method of any one of embodiments 1-30, wherein the particle is from 5 nm to 50 µm in a dimension.

32. The method of embodiment 31, wherein the dimension comprises a diameter.

33. The method of any one of embodiments 1-32, wherein the particle comprises an organic, inorganic, hybrid organic-inorganic, or polymeric particle.

34. The method of any one of embodiments 1-33, wherein the particle comprises a hollow particle, a solid particle, a porous particle, or a multi-layered particle.

35. The method of any one of embodiments 1-34, wherein the particle comprises a sphere, a rod, a triangle, a cylinder, a cube, a low symmetry shape, or another geometrical shape.

36. The method of any one of embodiments 1-35, wherein the particle comprises an anionic, cationic, or neutral charge.

37. The method of any one of embodiments 1-36, wherein the particle is surface modified with a small molecule, peptide, protein, antibody, aptamer, or a functional chemical group.

38. The method of any one of embodiments 1-37, wherein the particle comprises a nanoparticle, microparticle, micelle, liposome, iron oxide particle, graphene particle, silica particle, protein-based particle, polystyrene particle, silver particle, gold particle, quantum dot, palladium particle, platinum particle, titanium particle, or any combinations thereof.

39. The method of any one of embodiments 1-38, wherein the probe comprises a fluorophore.

40. The method of any one of embodiments 1-39, wherein the probe and the barcode are conjugated by a linker.

41. The method of embodiment 40, wherein the linker comprises a C3 linker, a C6 linker, a C12 linker, a C18 linker, a C36 linker, a peptide linker, a nucleic acid linker, a chemical linker, a PEG linker, a cleavable linker, or a non-cleavable linker.

42. The method of any one of embodiments 1-41, wherein the barcode comprises a nucleic acid molecule from 20 to 1000 nucleotides in length.

43. The method of any one of embodiments 1-42, wherein the biomolecule comprises a protein.

44. The method of embodiment 43, wherein the protein comprises a post-translational modification recognizable by the affinity reagent.

45. The method of any one of embodiments 1-42, wherein the biomolecule comprises a lipid, a nucleic acid, or a saccharide.

46. The method of any one of embodiments 1-45, wherein the sample comprises a biofluid.

47. The method of embodiment 46, wherein the biofluid comprises plasma, serum, urine, cerebrospinal fluid, synovial fluid, tears, saliva, whole blood, milk, nipple aspirate, ductal lavage, vaginal fluid, nasal fluid, ear fluid, gastric fluid, pancreatic fluid, trabecular fluid, lung lavage, sweat, crevicular fluid, semen, prostatic fluid, sputum, fecal matter, bronchial lavage, fluid from a swabbing, or a bronchial aspirant.

48. The method of any one of embodiments 1-45, wherein the sample comprises a fluidized solid, a tissue homogenate, or a cultured cell.

49. The method of any one of embodiments 1-48, further comprising performing a wash step after a) to wash away biomolecules not adsorbed to the particle, performing a wash step after b) to wash away unbound probes, or performing a combination of wash steps.

50. The method of any one of embodiments 1-49, wherein the assaying of c) comprises separating the probe from the biomolecule.

51. The method of any one of embodiments 1-50, wherein the assaying of c) comprises separating the barcode from the affinity reagent.

52. The method of any one of embodiments 1-51, wherein the assaying of c) comprises measuring a readout indicative of the presence, absence or amount of the barcode.

53. The method of any one of embodiments 1-52, wherein the assaying of c) comprises assaying for the presence or absence of the barcode.

54. The method of any one of embodiments 1-53, wherein the assaying of c) comprises assaying for an amount of the barcode.

55. The method of any one of embodiments 1-54, wherein the barcode corresponds to the biomolecule bound by the affinity reagent.

56. The method of any one of embodiments 1-55, further comprising contacting the probe with a secondary probe comprising a nucleotide that hybridizes with the barcode.

57. The method of embodiment 56, wherein the secondary probe comprises a detection modality.

58. The method of embodiment 57, wherein the detection modality of the secondary probe is fluorescent.

59. The method of embodiment 57 or 58, wherein c) comprises measuring a readout indicative of the presence, absence or amount of the detection modality of the secondary probe.

60. The method of any one of embodiments 57-59, wherein the secondary probe is present in a plurality of secondary probes comprising different tags and nucleotides that hybridize with different barcode sequences.

61. The method of any one of embodiments 1-60, further comprising performing mass spectrometry, chromatography, liquid chromatography, high-performance liquid chromatography, solid-phase chromatography, a lateral flow assay, an immunoassay, an enzyme-linked immunosorbent assay, a western blot, a dot blot, or immunostaining, or a combination thereof, on the biomolecule of the biomolecule corona or on one or more other biomolecules of the biomolecule corona.

62. The method of any one of embodiments 1-61, wherein the affinity reagent comprises the barcode.

63. A method of assaying biomolecules, comprising:
a) incubating a particle in a biological sample, thereby adsorbing biomolecules from the biological sample onto the particles to form biomolecule coronas;
b) incubating the particles with probes comprising (i) affinity reagents and (ii) barcodes, wherein the affinity reagents bind to biomolecules of the biomolecule coronas;
c) detecting the presence or amount of the barcodes of the probes comprising affinity reagents bound to biomolecules of the biomolecule coronas; and
d) identifying a biomolecule fingerprint associated with the biological sample based on the presence or amount of the barcodes.

64. The method of embodiment 63, further comprising identifying the presence or amount of the biomolecules of the biomolecule coronas based on the presence or amount of the barcodes.

65. The method of embodiment 64, wherein identifying the biomolecule fingerprint associated with the biological sample based on the presence or amount of the barcodes comprises identifying the biomolecule fingerprint based on the presence or amount of the biomolecules of the biomolecule coronas.

66. The method of any one of biomolecular 63-65, further comprising identifying a disease state associated with the biomolecule fingerprint.

67. The method of embodiment 66, wherein the disease state comprises a cancer, cardiovascular disease, endocrine disease, inflammatory disease, or neurological disease.

68. The method of embodiment 66 or 67, wherein identifying the disease state associated with the biomolecule fingerprint comprises applying a classifier to the biomolecule fingerprint.

69. The method of embodiment 68, wherein the classifier has been trained with data comprising the presence or amounts of barcodes of probes bound to biomolecule coronas of healthy or diseased subjects.

70. The method of any one of embodiments 63-69, wherein the particles comprise physiochemically distinct groups of particles.

71. A method of assaying a biomolecule in a sample, the method comprising:
a) incubating a particle in the sample thereby adsorbing biomolecules from the sample onto the particle to form a biomolecule corona;
b) incubating the particle with a probe comprising an affinity reagent that binds to a biomolecule of the biomolecule corona; and
c) assaying for the presence, absence or amount of the probe, thereby assaying for the presence, absence or amount of the biomolecule of the biomolecule corona.

72. The method of embodiment 71, wherein the probe comprises a detection modality.

73. The method of embodiment 72, wherein the detection modality is detectable optically, electrochemically, chemically, magnetically, chromatographically, by affinity capture, mass spectrometrically, or any combination thereof.

74. The method of embodiment 72 or 73, wherein the detection modality comprises a dye, a fluorescent tag, an electrochemically detectable tag, a magnetic tag, an affinity label, a polymer, a mass tag, or any combination thereof.

75. The method of any one of embodiments 71-74, wherein the probe is present in a plurality of probes.

76. A method of assaying a biomolecule in a sample, the method comprising:
a) incubating a particle in the sample thereby adsorbing biomolecules from the sample onto the particle to form a biomolecule corona;
b) incubating the particle with an affinity reagent that binds to a biomolecule of the biomolecule corona; and
c) assaying for the presence, absence or amount of the affinity reagent, thereby assaying for the presence, absence or amount of the biomolecule of the biomolecule corona.

77. The method of embodiment 76, wherein the affinity reagent comprises a nucleic acid.

78. The method of embodiment 77, wherein the affinity reagent comprises an aptamer.

79. The method of embodiment 78, wherein assaying for the presence, absence or amount of the affinity reagent comprises sequencing the aptamer.

80. The method of embodiment 78 or 79, wherein the aptamer binds comprises binding specificity for the biomolecule.

81. The method of any one of embodiments 76-80, wherein the biomolecule is more abundant in a sample of a subject having a first biological state than in a sample of a subject having a second biological state.

82. The method of any one of embodiments 76-81, wherein the affinity reagent has been subjected to error prone nucleic acid amplification.

83. The method of any one of embodiments 76-82, wherein the affinity reagent is present in a plurality or library of affinity reagents.

84. A method of assaying a biomolecule in a sample, the method comprising:
a) incubating a particle in the sample thereby adsorbing biomolecules from the sample onto the particle to form a biomolecule corona;
b) desorbing biomolecules of the biomolecule corona from the particle;
c) contacting the desorbed biomolecules with a probe comprising (i) an affinity reagent and (ii) a detection modality, wherein the affinity reagent binds to a biomolecule of the desorbed biomolecules; and
d) assaying for the presence, absence or amount of the detection modality of the probe comprising the affinity reagent, thereby assaying for the presence, absence or amount of the biomolecule of the desorbed biomolecules.

85. The method of embodiment 84, wherein the detection modality comprises a barcode.

86. The method of embodiment 84 or 85, further comprising binding the desorbed biomolecules to a substrate prior to d).

87. The method of embodiment 86, wherein the substrate has a flat surface to which the desorbed biomolecules are bound.

88. The method of embodiment 86 or 87, wherein the desorbed biomolecules are bound indirectly to the substrate.

89. The method of embodiment 88, wherein the desorbed biomolecules are bound to the substrate by capture moieties.

90. The method of embodiment 86 or 87, wherein the probe is bound to the substrate.

91. The method of any one of embodiments 86-90, further comprising releasing the desorbed biomolecules from being bound to the substrate prior to d).

92. The method of any one of embodiments 86-91, wherein the substrate comprises glass, a polymer, rubber, plastic, or a metal.

93. The method of any one of embodiments 86-92, further comprising releasing the desorbed biomolecules from being bound to the probe prior to d).

94. The method of any one of embodiments 86-92, wherein d) comprises assaying for the presence, absence or amount of the detection modality of the probe comprising the affinity reagent bound to the biomolecule of the desorbed biomolecules.

95. An assay method, comprising:
a) incubating a particle in a sample, thereby adsorbing biomolecules from the sample onto the particle to form a biomolecule corona;
b) incubating the biomolecules of the biomolecule corona with a substrate of a biomolecule of the biomolecule corona; and
c) measuring a reaction product of the substrate, thereby assaying for a presence, absence, or an amount of the biomolecule of the biomolecule corona.

96. The method of embodiment 95, further comprising incubating the particle with a probe comprising an affinity reagent that binds to the biomolecule of the biomolecule corona, and blocks formation of the reaction product from the substrate.

97. The method of embodiment 96, wherein the probe further comprises a barcode nucleotide sequence.

98. The method of embodiment 97, further comprising sequencing the barcode.

99. The method of embodiment 98, further comprising identifying the affinity reagent as an inhibitor of an enzyme activity of the biomolecule, based on the sequencing of the barcode.

100. An assay method, comprising:
a) flowing a sample over or through a matrix, thereby adsorbing biomolecules from the sample onto the matrix;
b) flowing a probe over or through the matrix, wherein the probe comprises (i) an affinity reagent and (ii) a barcode, and wherein the affinity reagent binds to a biomolecule of the adsorbed biomolecules; and
c) assaying for the presence, absence or amount of the probe, thereby assaying for the presence, absence or amount of the biomolecule of the adsorbed biomolecules.

101. The method of embodiment 100, wherein the matrix is semipermeable.

102. The method of embodiment 100 or 101, wherein the matrix comprises a porous material.

103. The method of any one of embodiments 100-102, wherein the matrix comprises a property comprising a charge, a hydrophobicity, or a surface functionalization.

### EXAMPLES

The following examples are illustrative and non-limiting to the scope of the devices, systems, fluidic devices, kits, and methods described herein.

### Example 1

### Affinity Reagents and Particles for Rapid Identification of Proteins

This example describes a method of coupling affinity reagents with particles of the present disclosure for rapid identification of a protein of interest. A particle of the present disclosure is incubated in a sample. Proteins in the sample adsorb to the particle surface to form a protein corona. Particles having a protein corona are further incubated with an affinity reagent. The affinity reagent is a peptide, protein, Fab, aptamer, scFv, full length antibody, small molecule, or any proteomic scaffold. The affinity reagent is, optionally, a system of two or more affinity reagents that interact with each (e.g., two antibodies bearing nucleic acid sequences that hybridize to each other). The affinity reagent is coupled to a detection modality. The detection modality involves amplification/sequencing (e.g., next generation sequencing), O-link, mass spectrometry, optical detection, fluorescent detection, etc.). Upon incubation of particles having the protein corona with the affinity reagent, in the presence of its target in the protein corona, the affinity reagent binds to the protein and, thereby, binds to the particle. The binding event is detected via the detection modality, thereby allowing for rapid identification of a protein of interest in the absence of identifying the protein by mass spectrometric analysis.

### Example 2

### Libraries of Affinity Reagents and Particle for Rapid Identification of Proteins

This example describes a method of coupling libraries of affinity reagents with particles of the present disclosure for rapid identification of a protein of interest. A particle of the present disclosure is incubated in a sample. Proteins in the sample adsorb to the particle surface to form a protein corona. Particles having a protein corona are further incubated with the library of affinity reagents. The affinity reagents are nucleic acid molecules, each of which include a unique barcode nucleotide sequence. Upon incubation of particles having the protein corona with the library of affinity reagents, in the presence of its target in the protein corona, an affinity reagent of the library binds to the protein and, thereby, binds to the particle. Optionally, the particles are separated from the library of affinity reagents. Optionally, solvents are added for dissolution of the particles, which do not affect the bound protein and affinity reagent. Amplification and sequencing reagents for the unique barcode nucleotide sequence are added and next generation sequencing is carried out, thereby detecting the binding event and allowing for rapid identification of a protein of interest in the absence of identifying the protein by mass spectrometric analysis.

### Example 3

### Detection of Cancer Biomarkers Using Biomolecule Coronas

This example covers cancer biomarker detection from biomolecule coronas with probe binding assay. Studies will be performed with a goal of evaluating the efficacy of detection of the presence of cancer biomarker proteins in biological samples taken from patients and used to form biomolecule coronas. Nucleolin, Tenascin-C, and epidermal growth factor receptor variant III (EGFR) are some examples of proteins that may be used for cancer detection, and may be found in biomolecule coronas. Nucleolin is a protein that is upregulated in some cancer cells, and may be present in nucleoli, nucleoplasm, cytoplasm, or on cell surfaces. Tenascin-C is an extracellular matrix protein that may be overexpressed during tissue remodeling processes, including tumor growth. The epidermal growth factor receptor (EGFR) is overexpressed in a variety of human epithelial tumors. However, its plasma concentration is often low, making detection and quantitation difficult. AS1411 G-rich DNA aptamer specifically recognizes nucleolin. TTA-1 aptamer shows strong binding with tenascin-C while A32 aptamer presents a strong affinity towards EGFR III. Nucleolin, tenascin-C and EGFR will be enriched on a particle surface, and subjected to aptamer binding analysis. Nucleolin, tenascin-C and EGFR abundances and abundance ratios will be used to distinguish plasma samples from healthy patients and cancer patients.

### Example 4

### Detection of Cancer Biomarkers Using DNA Aptamers

This example covers probe-based protein detection in biomolecule coronas. Protein samples taken from patients are diluted by TE buffer (10 mM Tris, 1 mM disodium EDTA, 150 mM KCl) with 0.05% CHAPS. Protein samples are also prepared from healthy people as a negative control group. To form protein coronas, 100 µL of NP suspension is mixed with 100 µL of a diluted sample in microtiter plates and incubated at 37° C. for 1 h with shaking at 300 rpm. Corona-coated NPs are separated from unbound and weakly bound proteins by a magnetic collection device. The corona-coated NPs are further washed with TE buffer three times with magnetic separation. The corona-coated NPs are further incubated with aptamer buffer solution at 37° C. for 1 h. The corona-coated NPs with aptamer binding are separated and washed. The bound DNA aptamers are extracted from corona-coated NP and subjected to amplification and further characterization to identify and quantify biomolecule corona proteins.

### Example 5

### Detection of Tumor-Associated Antigens Using Antibody

Cancer sera can contain antibodies which react with a unique group of autologous cellular antigens called tumor-associated antigens (TAAs). This study will determine whether a patient has TAAs based on specific antibody-antigen affinity interaction. Protein samples taken from patients are diluted by 0.01 M phosphate buffered saline (PBS) solution (0.15 M NaCl, 0.01 M Na₂HPO₄, and 1.7 mM NaH₂PO₄) with 0.05% CHAPS. Protein samples are also prepared from healthy people as a negative control group. To form protein coronas, 100 µL of NP suspension is mixed with 100 µL of a diluted sample in microtiter plates and incubated at 37° C. for 1 h with shaking at 300 rpm. Corona-coated NPs are separated from unbound and weakly bound proteins by a magnetic collection device. The corona-coated NPs are further washed with 0.01 M PBS solution three times with magnetic separation. The corona-coated NPs are further incubated with antibody buffer solution at 37° C. for 1 h. The corona-coated NPs with antibody binding are separated and washed. The bound antibodies are subjected to further characterization using fluorophore-tagged second antibodies to identify corona proteins.

### Example 6

### Detection of Cancer Biomarkers Using DNA Barcodes

This study relies on both specific antibody-antigen affinity interaction and detection efficiency of DNA barcode for the determination of the presence of tumor-associated antigens (TAAs) in a patient’s protein sample. To detect a specific protein in low amount in corona, bound DNA barcodes will be amplified by polymerase chain reaction (PCR) and identified by nucleic acid electrophoresis or next-generation sequencing (NGS) technique. Protein samples taken from patients are diluted by 0.01 M phosphate buffered saline (PBS) solution (0.15 M NaCl, 0.01 M Na₂HPO₄, and 1.7 mM NaH₂PO₄) with 0.05% CHAPS. Protein samples are also prepared from healthy people as a negative control group. To form protein coronas, 100 µL of NP suspension is mixed with 100 µL of a diluted sample in microtiter plates and incubated at 37° C. for 1 h with shaking at 300 rpm. Corona-coated NPs are separated from unbound and weakly bound proteins by a magnetic collection device. The corona-coated NPs are further washed with 0.01 M PBS solution three times with magnetic separation. The corona-coated NPs are further incubated with antibody buffer solution at 37° C. for 1 h. For this study, the antibodies will be conjugated with barcode DNAs. The corona-coated NPs with antibody binding are separated and washed. The bound antibodies with barcode DNAs are subjected to further characterization to identify corona proteins. To detect the barcode DNAs, DNAs will be extracted, and PCR will be performed in a 50 µL reaction containing 20 µL of DNA, 1 × Standard Taq Reaction Buffer (NEB, USA), 1.25 units of Taq DNA Polymerase (NEB), 200 µM dNTPs, and 0.2 µM of each primer. The cycling conditions will be in general one cycle of 95° C. for 30 s; 30 cycles of 95° C. for 15 s, 45-68° C. for 1 min, and 68° C. for 1 min; and one cycle of 68° C. for 5 min. The amplified barcode DNAs will be identified by gel electrophoresis.

### Example 7

### Detection of Cancer Biomarkers Using Proximity Extension Assay (PEA)

This example covers biomolecule corona analysis with probe-based proximity extension assays. PEA is based on pairs of antibodies that are linked to oligonucleotides having slight affinity to one another (PEA probes). Upon target binding the probes are brought in proximity, and the two oligonucleotides are extended by a DNA polymerase forming a new sequence that acts as a unique surrogate marker for the specific antigen. This sequence is typically quantified by quantitative real-time PCR (qPCR), where the number of PCR templates formed is proportional to the initial concentration of antigen in the sample. This study relies on both specific antibody-antigen affinity interaction and the proximity requirement for template formation to detect the presence of matched antigen biomarkers in a patient’s protein sample. Protein samples taken from patients are diluted by 0.01 M phosphate buffered saline (PBS) solution (0.15 M NaCl, 0.01 M Na₂HPO₄, and 1.7 mM NaH₂PO₄) with 0.05% CHAPS. Protein samples are also prepared from healthy people as a negative control group. To form protein coronas, 100 µL of NP suspension is mixed with 100 µL of a diluted sample in microtiter plates and incubated at 37° C. for 1 h with shaking at 300 rpm. Corona-coated NPs are separated from unbound and weakly bound proteins by a magnetic collection device. The corona-coated NPs are further washed with 0.01 M PBS solution three times with magnetic separation. For this study, two antibodies will be conjugated with each proximity probe DNA. The corona-coated NPs are further incubated with antibody buffer solution (PBS with 0.1% BSA), 0.3 µL Incubation Stabilizer (Olink Bioscience, Sweden) and 2.1 µL Incubation Solution (Olink Bioscience) overnight at 4° C. The corona-coated NPs with antibody binding are separated and washed. A combined extension and preamplification mix (96 µL) containing 10 µL MUX PEA Solution (Olink Bioscience), 0.5 units Pwo (DNA Gdansk, Poland), 1 µM forward and reverse universal preamplification primers, and 1 unit hot-start DNA polymerase are added to each reaction at room temperature. After mixing and a total 5-min incubation, the plate will be transferred to a thermocycler running an initial extension step to unite the two oligonucleotides (50° C., 20 min), immediately followed by a hot-start step (95° C., 5 min) and 17 cycles of amplification (95° C., 30 s; 54° C., 1 min; 60° C., 1 min). Amplification will be performed with universal flanking primers to amplify all sequences in parallel. Finally, 2.8 µL of the preamplification products are mixed with 7.2 µL buffer containing 5 µL MUX Detection Solution (Olink Bioscience), 0.071 units Uracil-DNA glycosylase (DNA Gdansk) used to digest the DNA templates and remaining universal primers, and 0.14 units hot-start polymerase. Five µL from the sample mix above is transferred to the sample inlet wells of a microfluidic real-time PCR chip (96.96 Dynamic Array IFC, Fluidigm Biomark). Five µL from respective well of an Assay Plate (Olink Bioscience) containing 9 µM sequence-specific internal detection primers, 2.5 µM molecular beacon in 1x DA Assay Loading Reagent (Fluidigm) are transferred to the assay inlet wells. The chip is run in a Biomark instrument with the following program: Thermal mix (50° C., 2 min; 70° C., 30 min; 25° C.; 10 min), Hot-start (95° C., 5 min), PCR Cycle 40 cycles (95° C., 15 s; 60° C., 1 min) according to the manufacturer’s guidelines. The amplified DNAs are identified by nanopore sequencing.

### Example 8

### Proteome Analysis

A proteome analysis method may combine Proteograph with an affinity reagent (e.g., a DNA encoded library (DEL)) binding assay (see, e.g., FIG. 7). A bare particle may be contacted with a sample. The particle following contact with the sample may form a biomolecule corona. The particle may subsequently be contacted by a library of affinity ligands, wherein a subset of members of the library of affinity ligands bind to biomolecules on the surface of the biomolecule corona, and the remainder are washed away. Bound affinity ligands may be desorbed from the corona and identified by NGS. The NGS can determine the identities and absolute quantities of each ligand present.

Mass spectrometric analysis of the biomolecule corona may be included. The biomolecules may be desorbed from the particle. Desorbed proteins may be digested into short peptides. The desorbed proteins may also be chemically treated (e.g., reduced) during this step. Short peptides may be analyzed by MALDI mass spectrometry, thus identifying the proteins present in the biomolecule corona formed during this assay.

### Example 9

### Determining a Biological State of a Patient

A method may include determining the biological state of a patient by contacting a sample from the patient with a naive library of affinity reagents (see, e.g., FIG. 8). Assays may be performed on biological samples from different patients. Bare particles may be contacted with samples. The particles following contact with the samples may form biomolecule coronas.

Each particle may be contacted with a library of affinity reagents. Specific targets for the library of affinity reagents may be unknown. Instead, a computational model may be trained to use the pattern of affinity reagent binding to identify the disease state of a subject. Affinity reagents which target biomolecules present on the surface of a biomolecule corona may bind to the biomolecule corona. Thus, an affinity reagent binding pattern to a biomolecule corona may be partially determined by the composition of the biomolecule corona. Unbound affinity reagents can be removed through multiple series of washes.

The remaining affinity reagents may be eluted from the surfaces of the biomolecule coronas and detected. Each type of affinity reagent has a unique absorbance profile, allowing the corona-bound affinity reagents to be quickly identified and quantified by absorbance within a diode array. Based upon the patterns of affinity reagent binding to each biomolecule corona, the computational algorithm may be able to identify the first patient as healthy, and the second patient as diabetic.

### Example 10

### Determining a Biological State of a Patient

A method may include determining the biological state of a patient by contacting a sample from the patient with an a priori library of affinity reagents (see, e.g., FIG. 9). The affinity reagents used in an assay may be evolved to bind specific biomolecular targets, so that the pattern of affinity reagent binding can be used to quantify the concentrations of specific biomolecules from a sample. Each type of affinity reagent may contain a unique identifier barcode, allowing each affinity reagent to be identified by NGS.

A bare particle may be contacted with a sample. The particle after it has been contacted the sample from the patient, may result in the formation of a biomolecule corona. The particle may be contacted by a library of affinity reagents. Affinity reagents that do not bind to the biomolecule corona may be removed from the sample through multiple wash cycles. The biomolecule corona may be bound affinity reagents being desorbed from the particle and identified by NGS. The affinity reagent binding pattern may be used to determine the concentrations of multiple proteins in the patient’s sample.

### Example 11

### Determining a Biological State of a Patient

A method may include determining the biological state of a patient using a particle array and an affinity reagent library (see, e.g., FIG. 10). The affinity reagent library may include DNA aptamers that are identifiable by NGS and that are each capable of binding multiple targets. While the identities of the DNA aptamers and their targets may be unknown, the affinity reagent library may undergo multiple rounds of evolution to differently bind to plasma samples from diabetic, pre-diabetic, and non-diabetic patients.

An array of particles that differ in composition and surface properties may be contacted with a sample from the patient. The differences in surface properties of the particles may lead to the formation of different biomolecule coronas on the particles. Each particle in the particle array may be separately contacted with an affinity reagent library. The affinity reagents that do not bind to the biomolecule coronas may be detected on an individual particle basis by NGS. The pattern of ligand non-binding may be used to fingerprint each sample, and to determine whether the patient that provided the sample is diabetic or pre-diabetic.

### Example 12

### Proteome Analysis

A proteome analysis method may combine Proteograph with a library of affinity ligands (e.g., a DNA encoded library (DEL)) binding assay (see, e.g., FIG. 11). A bare particle may be contacted with a sample. The particle following contact with a sample may form a biomolecule corona. The particle may subsequently be contacted by a library of affinity ligands, wherein a subset of members of the library of affinity ligands bind to biomolecules on the surface of the biomolecule corona, and the remainder are washed away. Bound affinity ligands may be desorbed from the corona and identified by NGS. The NGS can determine the identities and relative or absolute quantities of each ligand present.

Additional steps may involve mass spectrometric analysis of the biomolecule corona. A soft corona portion of a biomolecule corona may be desorbed into solution. Desorbed proteins may be digested into short peptides. The short peptides may be analyzed by MALDI mass spectrometry. A hard biomolecule corona may be desorbed from the particle. Desorbed proteins may be digested into short peptides. The short peptides may be analyzed by MALDI mass spectrometry. Thus, this assay may distinguish and independently identify biomolecules with different affinities for a particular particle’s biomolecule corona. In some cases, a particular biomolecule’s affinity for biomolecule corona binding may be dependent on the biological state associated with the sample. For example, a disease may lead to raised cell free DNA concentrations, which in turn may lower a particular protein’s affinity for binding to biomolecule coronas formed from that sample.

### Example 13

### Proteome Analysis

A proteome analysis method may combine Proteograph with a library of affinity ligands (see, e.g., FIG. 12). A particle may be contacted with a sample. The particle following contact with the sample may form of a biomolecule corona. Weakly bound biomolecules may be desorbed from the biomolecule corona. The desorbed biomolecules may be conjugated to capture moieties bound to a surface. The captured biomolecules may then be contacted by a library of affinity reagents. A subset of the affinity reagents may bind to captured proteins, and the remainder may be washed away. Bound ligands may be eluted from the captured proteins and identified by NGS.

### Example 14

### Analysis With Proximity Extension Assay

An analysis may include biomolecule collection on particles and a proximity extension assay (see, e.g., FIG. 13). A particle may be contacted with a sample. The particle following contact with the sample may form of a biomolecule corona. The particle may be contacted by a library of nucleic acid barcoded antibodies, wherein a subset of the nucleic acid barcoded antibodies bind to biomolecules on the surface of the biomolecule corona, and the remainder may be washed away. A pair of closely spaced antibodies with partially matching nucleic acid barcodes may be hybridized. The hybridized nucleic acid barcodes may undergo extension. The extension product may undergo amplification and sequencing.

### Example 15

### Analysis With Proximity Extension Assay

An analysis may include a proximity extension assay (see, e.g., FIG. 14). A particle may be contacted with a sample. The particle following contact with the sample may form of a biomolecule corona. The particle may then be contacted with a library of affinity reagents (e.g., a DEL or antibody library).

Multiple affinity reagents may be bound to the biomolecule corona. Each affinity reagent may include a target binding moiety and a single stranded nucleic acid barcode. The library of affinity reagents may include affinity reagents that bind small molecule targets and affinity reagents that bind peptide epitopes. When two affinity reagents with complementary nucleic acid barcodes bind within sufficient proximity (e.g., when a small molecule that is the target of a first affinity reagent is bound to a protein that is the target of a second affinity reagent), the barcodes may hybridize. This may enable extension of the nucleic acid barcodes. In a subsequent amplification step, nucleic acid barcodes that underwent extension may produce amplicons. The amplicons may be detected by NGS, indicating which pairs of affinity reagents bound to biomolecules are within close proximity within the sample.

### Example 16

### Proteome Analysis

A proteome analysis method may combine Proteograph with an affinity reagent binding assay (see, e.g., FIG. 15). A particle may be contacted with a sample. The particle following contact with the sample may form of a biomolecule corona. The particle may subsequently be contacted by a library of nucleic acid barcoded affinity reagents, wherein a subset of affinity reagents bind to biomolecules on the surface of the biomolecule corona, and the remainder may be washed away. The nucleic acid barcodes may be cleaved from the corona-bound affinity reagents coupled to collection and NGS. The remaining DEL members may be desorbed from the biomolecule corona. Biomolecule corona analysis may also be undergone using mass spectrometry.

### Example 17

### Proteome Analysis

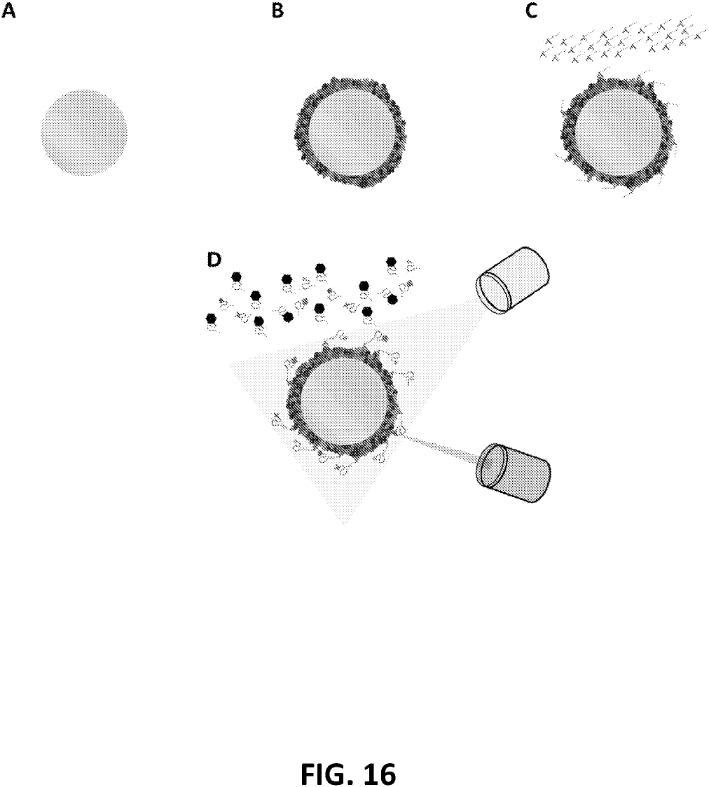

A proteome analysis method may be undergone that includes assaying biomolecules from a solution (see, e.g., FIG. 16). A particle may be contacted with a sample. The particle following contact with the sample may form of a biomolecule corona. A subset of affinity reagents from a library of nucleic acid barcoded affinity reagents may be bound to the surface of the biomolecule corona. The biomolecule corona may subsequently be contacted by a set of fluorescent probes that include single stranded nucleic acid barcodes. The fluorescent probes may hybridize to ligands with complementary nucleic acid barcodes. The bound fluorescent probes may then be fluorometrically detected.

### Example 18

### Analysis With Proximity Ligation Assay

An analysis may include a proximity ligation assay (see, e.g., FIG. 17). A particle may be contacted with a sample. The particle may be subsequently contacted with a library of affinity reagents (e.g., a DEL), resulting in a subset of the affinity reagents binding to biomolecules on the surface of the biomolecule corona.

Bound affinity reagents may include a biomolecule binding portion and a nucleic acid barcode. The nucleic acid barcodes may include double stranded regions with unique identifier sequences and sticky ends. When two affinity reagents are bound within sufficient proximity and the sticky ends of their nucleic acid barcodes are sufficiently complementary, their nucleic acid barcodes may be ligated.

The nucleic acid barcodes can be released from the biomolecule binding portions of the affinity reagents and then sequenced. Ligated barcode pairs may be read as a single sequence, indicating that the pair of affinity reagents that they originated from bound to targets that are within close proximity in the sample. Reads of non-ligated barcodes may indicate that a particular biomolecule is present in the sample, and that the biomolecule is not in close proximity to another biomolecular target recognized by the library of affinity reagents.

### Example 19

### Evolving an Aptamer Library

An method may include evolving a DNA aptamer library to preferentially recognize a particular disease state (see, e.g., FIG. 18). A DNA aptamer library may be contacted to a particle containing a biomolecule corona from a healthy patient. The library members that do not bind the sample may be collected, and then contacted to a particle containing a biomolecule corona from a diseased patient. Unbound library members may be washed away and the bound members may be collected, yielding a pool of DNA aptamers with a greater affinity for the diseased sample than the healthy sample. This pool of DNA aptamers may then be subjected to error prone PCR, and evolved through additional rounds of the selection assay until a DNA aptamer library with the ability to accurately distinguish healthy and disease state samples has been generated.

### Example 20

### Training a Model

An method may include training a computational model to distinguish a disease state from a healthy state (see, e.g., FIG. 19). A first particle may be contacted with a sample from a healthy patient (top row), and a second particle may be contacted with a sample from a patient carrying the disease (bottom row), leading to different biomolecule coronas on the two particles.

Particles may be contacted with a biological sample, resulting in the formation of a biomolecule corona. Each particle may be contacted with a library of affinity reagents. A subset of the affinity reagents may bind to each corona, while the remainder of the affinity reagents may be washed away. The corona-bound affinity reagents may be desorbed and sequenced. Mass spectrometry may be used to analyze the biomolecule coronas. The sequencing data (or the mass spectrometry data) may be used to train a computational algorithm (e.g., a neural network) to distinguish the disease state from the healthy state. This training method may not require any knowledge of the targets or binding affinities of the affinity reagents, but rather utilizes affinity reagent binding patterns to distinguish the biological states associated with the input samples.

### Example 21

### Obtaining Enzyme Activity or Inhibitors

An method may include identifying enzyme inhibitors or elucidating enzyme activity with a dual particle, affinity reagent assay (see, e.g., FIG. 20). A particle may be contacted with a sample. The particle, upon contact with a sample, may form a biomolecule corona. The particle may subsequently be contacted by a library of nucleic acid barcoded affinity reagents, wherein a subset of affinity reagents bind to biomolecules on the surface of the biomolecule corona, and the remainder may be washed away. The particle may then be contacted with a substrate of an enzyme present in the sample. A rate of the reaction can be monitored with a wide range of techniques including mass spectrometrically, spectroscopically, electrochemically, colorimetrically, or chromatographically. If the library contains an inhibitor affinity binding reagent, the enzyme reaction rate may be diminished. The identity of the inhibitory affinity binding reagent may be determined by sequencing its nucleic acid barcode. If a known enzyme inhibitor is provided in step C, this assay may be used to measure a particular enzyme’s activity (for example, whether a particular enzyme in a sample is activated). This type of assay may be incorporated into other types of assays, including Proteograph, to further elucidate a biological state. For example, diseases caused by constitutively activated ubiquitin ligases could be identified by parallel Proteograph and ubiquitin ligase activity assays.

### Example 22

### Affinity Reagent Evolution

An affinity reagent library evolution method that utilizes particle-based biomolecule collection may be performed (see, e.g., FIG. 21). A combinatorial library of polynucleotides may be randomly assembled from small nucleic acid library comprising a number of short nucleic acid sequences. The polynucleotide library may be contacted with a set of oligonucleotides coupled to reactive groups. If the sequence of a reactive-group bearing oligonucleotide is present in a polynucleotide from the combinatorial library, the two species may hybridize, and the reactive group may transfer from the oligonucleotide to the polynucleotide. Multiple contacting rounds may be used to generate complex sequences of reactive groups on each polynucleotide. The library of reactive group-bearing polynucleotides may be contacted to a particle covered with a biomolecule corona. A subset of polynucleotides may be coupled to sequences of reactive groups with affinities for a corona-bound biomolecule. The remaining polynucleotides may be washed away. The remaining nucleotides can optionally be digested, amplified, reassembled to form a new polynucleotide library, and subjected to additional rounds of evolution. This library evolution scheme can be used to generate affinity reagents with specificity for a particular biomolecule (e.g., ceruloplasmin) or disease state (e.g., Wilson’s disease). This method can also be used to generate a library with a plurality of affinity reagents targeting a plurality of biomolecules. This method can also be coupled to the method for identifying inhibitors for a particular enzyme.

### Example 23

### Assay Method

An method may include assaying a sample with a sensor array that uses semipermeable matrices as sensor elements (see, e.g., FIG. 22). The semipermeable matrices may be configured to collect biomolecules from a sample flowing through or past them. The biomolecule affinity of each semipermeable matrix may be dependent on its chemical and physical properties (e.g., charge, hydrophobicity, surface functionalization), as well as the sizes of its pores. Thus, two different semipermeable matrices may be produce different biomolecule corona signatures upon contacting the same sample.

A bulk fluid can either be flown through or over a semipermeable matrix. Both flow regimes may result in biomolecules adsorbing on or within the semipermeable matrix. Collected biomolecules can be eluted from a semipermeable matrix and subjected to further enrichment, treatment, and analysis. For example, a biomolecule collected on a semipermeable matrix may be eluted and analyzed by mass spectrometry or assayed with a library of affinity reagents.

### Example 24

### Parallel Assays

An method may include performing parallel assays on a single sample (see, e.g., FIG. 23). An array (e.g., a multi-well plate) may be obtained in which each well has a unique particle-type and affinity binding reagent library combination. Sample can be loaded into or incubated within each well. The contents of each well can then be washed, removing unbound biomolecules, cellular components, or affinity reagents, and leaving the biomolecule corona-coated particles and bound affinity reagents. This step may be performed with a filter-tipped aspirator. Each well may be loaded with reagents to cleave nucleic acid barcodes from the affinity binding reagents. The nucleic acid barcodes can be collected and sequenced. The biomolecules in each well may be analyzed by mass spectrometry. Each step may be automated, and multiple steps may be performed in parallel. For example, the multi-well plate may be loaded into a device that performs each well assay in parallel. Alternatively, the contents of each well may be individually aspirated into a separate container (e.g., a spin down column) for analysis.

### Example 25

### Assay Method

An method may include an assay in which a biological sample contacts a single type of particle under multiple distinct conditions (see, e.g., FIG. 24). Two particles may be provided in different conditions. One particle may be provided in a solution with a high ionic strength, a low pH, and a cool temperature, while another particle may be provided in a solution with a relatively low ionic strength, a neutral pH and a warm temperature. The conditions may be regulated throughout the assay so that pH, ionic strength and temperature remain constant.

The particles may be contacted with a biomolecule sample and form biomolecule coronas. Sizes and compositions of the biomolecule coronas may differ between particles in the separate conditions. Particles may be contacted by a library of affinity reagents. A pattern of affinity reagent binding may be affected by the solution conditions. This is may be in part due to differences in the biomolecule corona compositions, or in part due to changes in binding affinities due to the solution conditions. The affinity reagent binding profiles may be measured by NGS for each particle. The combination of affinity reagent binding profiles between all conditions assayed may be used to assign a biomolecule fingerprint to the sample.

### Example 26

### Biomolecule Corona Interrogation With a DNA-Encoded Library

This example outlines a method that was performed for identifying particle-adsorbed proteins with a large probe library. The library contained probes that included small molecule affinity reagents comprising functionalized pyrimidines, along with unique DNA barcodes. Three separate protein solutions, one containing Growth arrest - specific 6 (Gas6), one containing protein B, and one containing a 1:1 mixture of Gas6 and Angiogenin, were contacted to superparamagnetic particles for 1 hour at 37° C., facilitating protein adsorption to the particles. The superparamagnetic particles were magnetically immobilized and subjected to a series of three wash steps with HEPES buffer, thereby separating particle-adsorbed protein from unbound protein.

The superparamagnetic particle was resuspended in 150 µL of solution comprising the probe library, and incubated for 2.5 hours at ambient temperature. The superparamagnetic particle was again magnetically immobilized through a series of 3 HEPES buffer wash steps, separating unbound probes from the particle, and retaining probes bound to particle-adsorbed proteins.

The superparamagnetic particle was incubated with 200 units of T4 DNA ligase and 0.8 mM ATP for 24 hours. The superparamagnetic particle was then mixed with DNA polymerase I and dNTPs, and further incubated for 4 hours. Following incubation, the superparamagnetic particle was resuspended and magnetically separated. DNA barcodes of superparamagnetic particle-bound probes were collected from the particle, and quantified by using real-time PCR. The assay identified 8 probes which bound to the protein coronas generated in the Gas6 assay. The probe names and relative counts (e.g., the number of times that its barcode was observed) are provided in TABLE 2.

TABLE 2

Probe Binding To Gas6 Protein Coronas

| Probe Number | Counts |
|---|---|
| 1 | 10 |
| 2 | 10 |
| 3 | 10 |
| 4 | 11 |
| 5 | 11 |
| 6 | 13 |
| 7 | 13 |
| 8 | 47 |

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1. A method of assaying a biomolecule in a sample, the method comprising:
a) incubating a particle in the sample, thereby adsorbing biomolecules from the sample onto the particle to form a biomolecule corona;
b) incubating the particle with a probe comprising (i) an affinity reagent and (ii) a barcode, wherein the affinity reagent binds to a biomolecule of the biomolecule corona; and
c) assaying for the presence, absence or amount of the probe, thereby assaying for the presence, absence or amount of the biomolecule of the biomolecule corona.

2. The method of claim 1, wherein the affinity reagent comprises an antibody, a peptide, a nucleic acid ligand, a Fab, a Fab2, an scFv, an scFab, an aptamer, a polypeptide ligand scaffold, a ligand, or a chemical moiety.

3. The method of claim 2, wherein the peptide comprises an adnectin, abamer, affibody, or nanobody.

4. The method of claim 1, wherein the affinity reagent is from about 1 nm to about 35 nm in a dimension.

5. The method of claim 1, wherein the affinity reagent comprises a molecular mass from 200 Da to 200 kDa.

6. The method of claim 1, wherein the barcode comprises a single stranded nucleic acid, a double stranded nucleic acid, or a sticky end of a nucleic acid.

7. The method of claim 1, wherein the probe is present in a plurality of probes.

8. The method of claim 7, wherein the plurality of probes comprise different affinity reagents.

9. The method of claim 7, wherein the plurality of probes comprise a library of barcodes.

10. The method of claim 7, wherein each probe of the plurality of probes comprises a unique barcode.

11. The method of claim 9, wherein the library of barcodes comprises from 50 to 10¹⁰ distinct barcodes.

12. The method of claim 9, wherein the library of barcodes comprises a combinatorially generated nucleic acid library.

13. The method of claim 9, wherein the library of barcodes comprises double stranded DNA barcodes.

14. The method of claim 9, wherein the barcodes comprise barcode nucleotide sequences.

15. The method of claim 14, wherein affinity reagents of the plurality of probes bind different biomolecules, and wherein different biomolecules may be identified by the barcode nucleotide sequences of probes that bind to the different biomolecules.

16. The method of claim 15, wherein probes comprising affinity reagents that bind a biomolecule include a first barcode nucleotide sequence, and probes comprising affinity reagents that bind another biomolecule include a second barcode nucleotide sequence.

17. The method of claim 7, wherein a first probe of the plurality of probes comprises a first affinity reagent that binds a first biomolecule, and a second probe of the plurality comprises a second affinity reagent that binds a different region of the first biomolecule.

18. The method of claim 7, wherein a first probe of the plurality of probes comprises a first affinity reagent that binds a first biomolecule, and a second probe of the plurality of probes comprises a second affinity reagent that binds a second biomolecule in close proximity with the first biomolecule.

19. The method of claim 17, wherein a barcode of the first probe hybridizes with a barcode of the second probe.

20. The method of claim 19, further comprising extending the 3′ ends of the hybridized barcodes of the first and second probes.

21. The method of claim 19, wherein the barcodes of the first and second probes comprise sticky ends that hybridize together, and further comprising ligating the sticky ends.

22. The method of claim 14, wherein the assaying of c) comprises sequencing the barcode nucleotide sequences.

23. The method of claim 14, wherein the barcode nucleotide sequences comprise primer sequences.

24. The method of claim 14, wherein the assaying of c) comprises amplification.

25. The method of claim 24, wherein the barcode nucleotide sequences or a segment of the barcode nucleotide sequences is amplified prior to sequencing.

26-30. (canceled)

31. The method of claim 1, wherein the particle has a diameter from 5 nm to 50 µm in a dimension.

32. The method of claim 1, wherein the particle comprises an organic, inorganic, hybrid organic-inorganic, or polymeric particle.

33. The method of claim 1, wherein the probe comprises a fluorophore.

34. The method of claim 1, wherein the probe and the barcode are conjugated by a linker.

35. The method of claim 1, wherein the biomolecule comprises a protein, a lipid, a nucleic acid, or a saccharide.

36-38. (canceled)
